(19) United States
(12) Patent Application Publication
(10) Pub. No.: US 2005/0090641 A1
(43) Pub. Date: Apr. 28, 2005

(54) Self-assembling polymers, and materials fabricated therefrom

(76) Inventors: Regina Valluzzi, Somerville, MA (US); David Kaplan, Concord, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/490,799

(22) PCT Filed: Oct. 2, 2002
(86) PCT No.: PCT/US02/31375

Related U.S. Application Data
(60) Provisional application No. 60/326,743, filed on Oct. 2, 2001.
(60) Provisional application No. 60/385,809, filed on Jun. 4, 2002.

(51) Int. Cl. C07K 14/47; C08G 63/48; C08G 63/91

(52) U.S. Cl. 530/350; 525/54.1

(57) ABSTRACT

The invention features miniblock polymers containing solubilizing blocks and folding blocks, wherein the miniblock polymers in solution can self-fabricate to form materials having a long-range order. Also disclosed are rigid well-fabricated materials and methods of using these materials.

### GOVERNMENT SUPPORT

This invention was made with government support under Grant NAG8-1699 awarded by NASA; and Grants DMR-0090384 and BES-9727401 awarded by the National Science Foundation. Therefore, the government has certain rights in the invention.

### BACKGROUND OF INVENTION

Much of biology is based on the special properties of macromolecules. Self-assembly of biological macromolecules commonly occurs in nature. For instance, surfactant molecules such as phospholipids self-assemble to form bi-layered membranes, unilamellar vesicles, multilamellar vesicles, tubules, and micelles, which are of great importance to the functions of organisms. See, e.g., J. Schnur, Science, 1993, 262,1670; and S. A. Safran, Statistical Thermodynamics of Surfaces, Interfaces, and Membranes, Addison-Wesley, New York, 1994.

Liquid crystal molecules are similar to naturally occurring biological macromolecules, in that they are also macromolecules and can self-assemble. A liquid crystal is a thermodynamically stable phase characterized by anisotropy of properties without the existence of a three-dimensional crystal lattice, generally lying in the temperature range between the solid and isotropic liquid phase. Liquid crystal molecules can self-assemble into domains having less order than a crystal, but more order than a conventional liquid. Because liquid crystals are liquids, their molecules can be oriented in a straightforward manner.

Because of its supermolecular order, the three-dimensional materials have enhanced infrared (IR) absorption compared to the polymers from which they are made. When the self-assembled material described above is applied to the surface of a device the IR absorption is enhanced. The device can be an IR sensor or an IR filter. Current methods of enhancing infrared absorption have been limited to coatings and shields, which normally require careful handling. Examples of such coatings include those made of anodized aluminum, metal oxides (e.g., ZrO₂, Na₂O, and Fe₃O₄), organic-inorganic complexes (e.g., a dithio-nickel complex), or thermally reactive polymers (e.g., an acrylate copolymer). See, e.g., U.S. Pat. Nos. 4,111,762, 4,525,462, 6,217,796, 6,177,182, and 6,124,425.

### SUMMARY OF INVENTION

In certain embodiments, the present invention is drawn to a miniblock polymer comprising one or more self-fabricating blocks and one or more solubilizing blocks, wherein the miniblock polymer has a molecular weight of about 1,000 to about 300,000 and, in solution, can self-fabricate to form a three-dimensional material having a long-range order, and wherein the miniblock polymer has a glycine content of at least 20%.

In a further embodiment, the self-fabricating block causes the miniblock polymer to form a helix.

In a further embodiment, the miniblock polymer comprises two solubilizing blocks as end blocks, and the self-fabricating block as a core block.

In a further embodiment, the self-fabricating block contains glycine at every third position of the sequence of the block.

In a further embodiment, the miniblock polymer is a polypeptide.

In a further embodiment, the miniblock polymer comprises at least two solubilizing blocks and one self-fabricating block.

In a further embodiment, the self-fabricating block has a glycine content of 30-80%.

In a further embodiment, the self-fabricating block contains glycine every third monomer, contains greater than 25% amino acids, and has a total block length of more than 6 monomers.

In a further embodiment, the self-fabricating block undergoes a random coil to helix transition.

In a further embodiment, the self-fabricating block is a confirmation selecting block imparting either hydrophobicity or hydrophilicity to the overall miniblock polymer and stabilizing secondary structure intermediates between a twofold extended helix and a threefold extended helix.

In a further embodiment, the self-fabricating block contains native sequences found in spider silks, prions, or amyloids.

In a further embodiment, the glycine exists in a glycine block selected from the group consisting of (GX)ₙ, (GGX)ₙ, (XG)ₙ, (XGG)ₙ, (GXX)ₙ, (XXG)ₙ, (GGXGXX)ₙ, (GXGXGGXGXX)ₙ, and combinations thereof, wherein X is a non-glycine amino acid, and n is an integer from 1 to 5 inclusive.

In a further embodiment, the glycine exists in a glycine block selected from the group consisting of GAGAGS, GAGAGY, and (G[AVLI]G[AVLI])ₙG[YST]), wherein n is an integer from 1 to 5 inclusive.

In a further embodiment, the self-fabricating block has a peptide sequence selected from the group consisting of:
GGAGGGGTGGLGSGGAGAGGLGGGGAG;
GDVGGAGATGGS;
GNVGGAGASGGS;
GAIGGVGATGGS;
SGAGVGRGDGSGVGLGSGNG;
GPGGTGPGQQGPGGTW;
GPGNNGPGGYGPGNNGPSGPGSA;
DPGVYGPSGNAPGVYGPSGQGAGAGS;
GVGVGS;
GIGIGS;
GVGGGY;
GAGAGY;
GAGAGD;
SGRGGLGGQGAGGGAGQGGYGGLGSQG;
MKHMAGAAGAVVGGLGGYMLGSAM; and
GAGAGT.

In a further embodiment, the miniblock polymer has a peptide sequence selected from the group consisting of:
(E)₅G(C)₂(GAPGPP)₅(C)₂G(E)₅;
(E)₅GCCE(GAPGPP)₂GAPGPR-GDPGPP-GAPGPP(C)₂G (E)₅;
(E)₂CERGDE(GAPGPP)₅ERGDEC(E)₂;
(E)₅(GAPGPP)₂GCPGPP(GAPGPP)₂(E);
(E)₂G(C)₄(GAPGPP)₅(C)₄G(E)₂;
(E)₂G(C)₄(GVPGPP)₅(C)₄G(E)₂;
(E)₃(GCPGPC)₆(E)₃;
(E)₃(GPAGPP)₄(E)₃;
(E)₃(GAOGPO)₄(E)₃;
(E)₃(GVOGPO)₄(E)₃;
(E)₅(GPPGVP-GPPGPS-GPPGVP-GSPGPP-GPVGPS-GPP)(E)₅;
(E)₅(GAPGPO)₆(E)₅;
(E)₅(GVOGPO)₆(E)₅;
(K)₅(GAPGPPGDP)₄(K)₅;
N₅(GPAGPP)₆N₅;
N₅(GAPGPP)₆N₅;
N₅(GPVGPP)₆N₅;
N₅(GVPGPP)₆N₅;
NGSNN(GAPGPP)₆ NGSNN;
N₅(GPAGPP)₃GPRGDP(GAPGPP)₃N₅;
(E)₅(GVPGPV)₆(E)₅;
(E)₅(GVPGPA)₆(E)₅;
(GAPGPP)ₙ;
(E)₅(GVPGPP)₆(E)₅;
(E)₅(GLPGPP)₆(E)₅;
(E)₅(GIPGPP)₆(E)₅; and
(K)₅(GPPGPPGDP)₄(K)₅.

In a further embodiment, the miniblock polymer has a peptide sequence selected from the group consisting of:
SAASAASAASAASAASAA;
SALSVASIASALSVASIA;
YALSVATIAYALSVATIA;
AAAAYAAAAYAAAAYAAAAY;
AAAAYAAYAAYAAYAAY;
AAASSIIIAAASIIIAAASS;
(E)₃(A)15(E)₃;
(E)₃(A)₂₀(E)₃;
(E)₃(A)₂₂(E)₃;
(EAAAK)₄; and
(EAAAK)₅.

In a further embodiment, the miniblock polymer has a peptide sequence selected from the group consisting of:
GIGIGS;
(E)₅(GAGAGD)₄(E)₅;
(E)₅(GVGVGD)₄(E)₅;
(E)₅(GLGLGD)₄(E)₅;
(E)₅(GIGIGD)₄(E)₅;
(E)₅(GVGVGY₄(E)₅;
(E)₅(GKGAGD)₄(E)₅;
(E)₅(GAGAGT)₄(E)₅;
(E)₅(GGAGGA)₄(E)₅;
(E)₅(GGAGGT)₄(E)₅;
(E)₅(GGAGGD)₄(E)₅;
SGAGVGRGDGSGVGLGSGNG;
GDVGGAGATGGS;
GNVGGAGASGGS;
GGAGGGGTGGLGSGG;
GGAGGGGTGGLGSGGAGAGGLGGGGAG;
GPGGTGPGQQGPGGTW;
GPGNNGPGGYGPGNNGPSGPGSA;
DPGVYGPSGNAPGVYGPSGQGAGAGS;
(E)₅(GAGAGS)₄(E)₅;
(E)₅(GVGVGS)₄E)₅;
(E)₅(GIGIGS)₄E)₅;
(E)₅(GVGVGY)₄(E)₅;
(E)₅(GAGAGY)₄(E)₅;
(E)₅(GAGAGD)₄(E)₅;
(E)₄(GAGAGS)₂(E)₄(GAGAGS)(E)₄;
(E)₄(GAGAGS)(E)₄(GAGAGS)(E)₄;
(E)₆(GAGAGD)₃(E)₆;
(E)₆(GVGVGS)₃(E)₆;
(E)₆(GIGIGS)₃(E)₆;
(E)₆(GVGVGY)₃)₆;
(E)₆(GAGAGS)₃(E)₆;
(E)₅(GPGGYGPGQQGPGGY)(E)₅;
(E)₅(GPGQQGPGGYGPGQQGPSGPGSA)(E)₅;
(E)₅(DPGVY-GPSGQ-DPGVY-GPSGQ)(E)₅;
GAIGGVGATGGS;
(R)₃(GGAGQGGYGGLGSQGAGRGGLGGQGAG)(R)₃;
GVGVGS;
(E)₅(SGAGVG-RGDGSGV-GLGSGNG)₂(E)₅;
(E)₅(GDIGGV-GATGGS)₂(E)₅;
(E)₅(GNVGGA GASGGS)₂(E)₅;
(E)₅(GVDGGA-GATGGS)₂(E)₅;
GVGGGY;
GAGAGY;
GAGAGD;
SGRGGLGGQGAGGGAGQGGYGGLGSQG;
GAGAGT;
(SGAGVGRGDGSGVGLGSGNG);
(R)₃(GGAGQGGYGGLGSQGAGRGGLGGQGAG)(R)₃;
(E)₅(GDVGGAGATGGS)₂(E)₅;
(E)₅(GDVGGAGASGGS)₂(E)₅;
(E)₅(GDIGGVGATGGS)₂(E)₅;
(E)₅(GPGGYGPGQQGPGGY)(E)₅;
(E)₅(GPGQQ-GPGGY-GPGQQ-GPSGPG-SA)(E)₅; and
(E)₅(DPGVY-GPSGQ-DPGVY-GPSGQ)(E)₅.

In a further embodiment, the miniblock polymer has a peptide sequence selected from the group consisting of:
SGRGGLGGQGAGMAAAAMMGGAGQGGYGGLGSQG;
MKHMAGAAGAVVGGLGGYMLGSAM;
MDAEFRHDSGYEVHHQKLVFFAEDVGSNKGAIIGLMVGGVIATVIVITLVM; and
(E)₄(VSSTGSTSNTDSSSKSAGSRTSGGTSTYGYSSSHRGGS)(E)₄.

In a further embodiment, the miniblock polymer contains a peptide sequence selected from the group consisting of:
DEDEDED;
GAGAGSGAGAGSGAGAGS;
GAPGPP;
GPAGPP;
GPAGPP;
GAPGPP; and combinations thereof.

In a further embodiment, the miniblock polymer has the peptide sequence: (EDE)₅(GAGAGS)₄RGDS(GAGAGS)₄PGP(GAGAGY)₂PGP(GAGAGS)₂(EDE)₄.

In a further embodiment, the miniblock polymer has the peptide sequence: NYNS(GCCCG)₃NSYS(GPGGTGPGQQGPGGTW)₂GPG(GVGVGSGAGAGD)₃.

In a further embodiment, the miniblock polymer has the peptide sequence: GEGP MKHMA EGPG (GAGAGYGAGY) GPG GESYRGDGSG.

In a further embodiment, the miniblock polymer has the peptide sequence: GPG (GVPGVAGGP)₃SYSSNR.

In a further embodiment, the miniblock polymer has the peptide sequence selected from the group consisting of:
(E)₅(GVPGPV)₆(E)₅;
(E)₅(GVPGPA)₆(E)₅;
(E)₅(GLPGPP)₆(E)₅;
(E)₅(GIPGPP)₆(E)₅;
(K)₅(GPPGPPGDP)₄(K)₅;
(K)₅(GAPGPPGDP)₄(K)₅;
(GSPGPP)ₙ; and
(GAGAGS)ₙ; wherein n is an integer from 1 to 5 inclusive.

In another embodiment, the present invention is drawn to a miniblock polymer comprising one or more self-fabricating blocks and one or more solubilizing blocks, wherein the miniblock polymer has a molecular weight of about 1,000 to about 300,000 and, in solution, can self-fabricate to form a three-dimensional material having a long-range order, and wherein the miniblock polymer has a glycine content of at least 20%, and wherein the miniblock polymer further comprises one or more blocks for triggering self-fabrication by external or environmental conditions.

In a further embodiment, the external or environmental condition is selected from the group consisting of temperature, light, redox chemistry, enzymatic reactions, and pH.

In a further embodiment, the self-fabricating block causes the miniblock polymer to form a helix.

In a further embodiment, the miniblock polymer comprises two solubilizing blocks as end blocks, and the self-fabricating block is within the core block.

In a further embodiment, the self-fabricating block contains glycine at every third position of the sequence of the block.

In a further embodiment, the miniblock polymer is a polypeptide.

In a further embodiment, the miniblock polymer comprises at least two solubilizing blocks and one self-fabricating block.

In a further embodiment, the self-fabricating block has a glycine content of 30-80%.

In another embodiment, the present invention is drawn to a miniblock polymer comprising one or more self-fabricating blocks and one or more solubilizing blocks, wherein the miniblock polymer has a molecular weight of about 1,000 to about 300,000 and, in solution, can self-fabricate to form a three-dimensional material having a long-range order, and wherein the miniblock polymer has a glycine content of at least 20%, and wherein the miniblock polymer further comprises a block for incorporating turns in the polymer or for providing sites for chemical modifications.

In a further embodiment, the self-fabricating block causes the miniblock polymer to form a helix.

In a further embodiment, the miniblock polymer comprises two solubilizing blocks as end blocks, and the self-fabricating block is within the core block.

In a further embodiment, the self-fabricating block contains glycine at every third position of the sequence of the block.

In a further embodiment, the miniblock polymer is a polypeptide.

In a further embodiment, the miniblock polymer comprises at least two solubilizing blocks and one self-fabricating block.

In a further embodiment, the self-fabricating block has a glycine content of 30-80%.

In another embodiment, the present invention is drawn to a miniblock polymer comprising one or more self-fabricating blocks, one or more solubilizing blocks, one or more blocks for triggering self-fabrication by external or environmental conditions, and one or more blocks for incorporating turns in the polymer or for providing sites for chemical modifications, wherein the miniblock polymer has a molecular weight of about 1,000 to about 300,000 and, in solution, can self-fabricate to form a three-dimensional material having a long-range order, and wherein the miniblock polymer has a glycine content of at least 20%.

In a further embodiment, the self-fabricating block causes the miniblock polymer to form a helix.

In a further embodiment, the miniblock polymer comprises two solubilizing blocks as end blocks, and the self-fabricating block is within the core block.

In a further embodiment, the self-fabricating block contains glycine at every third position of the sequence of the block.

In a further embodiment, the miniblock polymer is a polypeptide.

In a further embodiment, the miniblock polymer comprises at least two solubilizing blocks and one self-fabricating block.

In a further embodiment, the self-fabricating block has a glycine content of 30-80%.

In another embodiment, the present invention is drawn to a self-fabricated material having a three-dimensional structure, comprising a plurality of the miniblock polymers.

In a further embodiment, the present invention is drawn to a self-fabricated material having a three-dimensional structure, comprising a plurality of the miniblock polymers, and wherein the self-fabricated material has on its surface a patterned film of inorganic, organometallic, or optically active material.

In another embodiment, the present invention is drawn to a method of controlled delivery of a drug, the method comprising incorporating a drug within the self-fabricated material having a three-dimensional structure, comprising a plurality of the miniblock polymers, and administering to a subject in need thereof the self-fabricating material incorporating the drug.

In a further embodiment, the drug is incorporated within layers of the self-fabricating material.

In another embodiment, the present invention is drawn to a method of modifying the optical response of a device in the near to mid infrared wavelength range, the method comprising applying a self-fabricated material having a three-dimensional structure and comprising a plurality of the miniblock polymers to the surface of the device.

In another embodiment, the present invention is drawn to a miniblock polymer comprising multimeric associated helical segments and non-helical segments, wherein the polymer in solution can self-assemble to form a material having a long-range order and at least one of the helical segments comprises a peptide sequence having a glycine (G) residue at every third position.

In a further embodiment, the non-helical segments are hydrophobic.

In a further embodiment, the non-helical segments comprise glutamic acid.

In another embodiment, the present invention is drawn to a self-fabricated material having a long-range order in the solid glassy state, wherein the material is prepared from a miniblock polymer comprising helical and non-helical segments.

### BRIEF DESCRIPTION OF THE DRAWINGS

FIG. 1 depicts schematic diagrams of two simple liquid crystalline arrangements. FIG. 1A shows a cholesteric arrangement that is essentially “one dimensionally ordered.” FIG. 1B shows a smectic arrangement of rod-like molecules assembled into layers.

FIG. 2 depicts an illustration of the sequence of a folding block, in which X and Y stand for conformation-selecting blocks and G stands for a glycine block. Hydrophobic glycine patterns (Gly-X) and hydrophilic patterns (Gly-Z) are combined to make a helical “A” block. Sequences are numbered from left to right to correspond to the spiral (helix) shown.

FIG. 3 depicts diagrams of a helical polymer that has a molecular pitch and is inherently chiral. FIG. 3A shows a schematic diagram of a helical biopolymer, and FIG. 3B shows chiral twisting and supermolecular twisting in the biopolymer.

FIG. 4 depicts field emission scanning electron microscope (FESEM) images of a repetitive peptide having a smectic, layered structure. FIG. 4B shows a different region of the same film shown in FIG. 4A.

FIG. 5 depicts a fourier transform infrared (FTIR) spectrum of a typical collagen-like molecular helix.

FIG. 6 depicts an FTIR spectrum (transmission and reflection FTIR) of a nonrepetitive collagen-like peptide.

FIG. 7 depicts a diagram of a miniblock oligomer in a layered structure. The self-fabricating blocks (gray, which are oriented along helical axis) and the solubilizing blocks (red, which form unoriented sublayers) in this triblock example result in chemically distinct nanolayered regions throughout the material.

FIG. 8 depicts a diagram of a miniblock oligomer with three blocks. The self-fabricating block is represented as a smooth cylinder. Solubilizing blocks are shown as flexible lines at the ends of the cylinder.

FIG. 9 depicts diagrams of nanopatterned thin films of a miniblock oligopeptide with 5 nanometer chemical pattern exposed every 30 nanometers. FIG. 9A is a low magnification image of an oligopeptide with a self-fabricating block based on a silkworm silk motif. FIG. 9B is a high magnification image of films from an oligopeptide with a spider-silk based motif as a self-fabricating block.

FIG. 10 depicts a schematic diagram of a miniblock polypeptide with a complex block structure.

FIG. 11 depicts representations of micrographs of self-fabricated textured “tapes” from a peptide with sequence (Glu)₅(Ser-Gly-Ala-Gly-Val-Gly-Arg-Gly-Asp-Gly-Ser-GlyVal-Gly-Leu-Gly-Ser-Gly-Asn-Gly)₂(Glu)₅ (SEQ ID NO: 1). FIG. 11A is an optical micrograph showing a 10-15 micron texture that persists through the material thickness. FIG. 11B is a polarizing optical microscope image revealing patterned birefringence, indicating that the topographic texture is due to a changing material orientation. FIG. 11C is a scanning electron microscope (SEM) image showing the topographic structure of the tape.

FIG. 12 depicts representations of self-fabricated tapes of (Glu)₅(Ser-Gly-Ala-Gly-Val-Gly-Arg-Gly-Asp-Gly-Ser-GlyVal-Gly-Leu-Gly-Ser-Gly-Asn-Gly)₂(Glu)₅ (SEQ ID NO: 1). FIG. 12A shows helices arranged in layers, layers form a superstructure that can orient relative to precipitate exterior or another surface. FIG. 12B shows that the self-limited width and thickness of the fibers form the largest length scale in the hierarchy. FIG. 12C shows a 3-micron subtexture within the ridges of the 40-micron texture. FIG. 12D shows a submicron texture of inclined sheets or layers.

FIG. 13 depicts A) an optical micrograph of tris-Ru(II)bipyridil chloride hexahydrate (Rubipy) crystals. The racemic compound is weakly birefiingent and has a hexagonal crystal structure. FIG. 13B depicts a polarizing optical micrograph of the same. FIG. 13C depicts a typical hexatic liquid crystalline structure from a collagen miniblock oligopeptide (E₅[GPAGPP]₆E₅). The peptide is weakly birefringent and peptide liquid crystals appear in “black and white” in the polarizing optical microscope.

FIG. 14 depicts peptide liquid crystals with Rubipy. A portion of the compound is incorporated into the peptide liquid crystals and is co-oriented. There is excess orange racemic ruthenium compound excluded from the liquid crystals.

FIG. 15 depicts: A) left x-ray pattern of a beam path through a cross section of pure peptide flake (E₅[GSPGPP]₆E₅ dried at 3° C.), right x-ray pattern of a beam path through face of the flake; B) x-ray pattern of E₅[GSPGPP]₆E₅ peptide dried at 3° C. with Rubipy.

FIG. 16 depicts an x-ray pattern of crystals prepared by a peptide+Rubipy solution dried at 1° C.

### DETAILED DESCRIPTION

Overview

One aspect of the invention relates to miniblock polymers, and more particularly to miniblock polymers that can self-assemble into three-dimensional materials through liquid crystalline behavior.

In part, this invention is based on the discovery that by selecting specific monomers that have a particular chirality, arranging the monomers in a particular sequence, and combining sufficient monomers to achieve specific molecular weights, one can create so-called “miniblock” polymers that have a specific overall structure and composition. These miniblock polymers can be used to prepare long-range ordered fluids (i.e., liquid crystals) in a variety of phases or forms, which can then undergo very specific structural transitions to form rigid materials. Such structural transitions can be transitions in folding, conformation, aggregation, or super-secondary structure. Additionally, the liquid crystals can be easily solidified without losing the liquid crystalline order. The long-range order of the ordered fluids provide enhanced properties, such as infrared (IR) absorption. The polymers can also include miniblocks that can be used to selectively trigger self-assembly.

Each of the new polymers consists of multiple miniblocks arranged in a long-range pattern referred to as a “block architecture” or “block pattern.” By creating the block architecture with miniblocks that form precise intermolecular interactions (e.g., chemical bonds, ionic bridges, and hydrogen bonding) and with miniblocks that are charged or attractive (e.g., in a polar solvent), a new polymer that can fold, or undergo some other transition, is obtained. Similarly, an identical overall block architecture having different detailed individual block chemistries can be used to obtain polymers that aggregate, undergo a coil-helix conformation transition, or which combine different self-fabricating blocks and transition types. For the polymers including charged miniblocks, the pH and polarity of their solutions determine the degree of ionization, and subsequently whether the ends are attractive (non-ionized) or repulsive (ionized and charged).

When the new miniblock polymers are unfolded they are flexible and exhibit surfactant-like behavior and ordered fluid formation. They can thus form micelles, micro-emulsions, and surfactant phases. By contrast, folded miniblock polymers are rigid, and exhibit new features (e.g., new geometry, diffusion behavior, exterior accessible chemistry, biological activity, and new optical and electronic properties) and a variety of possible liquid crystalline packing and phase behaviors.

Some of the new miniblock polymers have at least two chemically distinct miniblocks arranged in a pattern. These two types of miniblocks impart different functions to the new miniblock polymers. At least one of the two miniblocks is a heteroblock and at least one of the miniblocks, which may or may not be a heteroblock, exists in a rigid chiral structure after a folding transition. One of the miniblocks can be a solubilizing block, while the other can be a conformation-forming or fabricating miniblock.

Because of the presence of the fabricating hetero miniblocks, these new miniblock polymers can self-fabricate into three-dimensional materials having a patterned long-range order. The long range ordered structures formed by the miniblock polymers can be translationally periodic, as in a crystal. In addition, long range ordered structures can consist of a simple geometric pattern or element, with microscale to nanoscale dimensions, which is repeated throughout the material. In terms of precisely patterned, precise location (and often orientation) of molecular subdomains is a characteristic feature. In other embodiments, the rotation and translation or more complex descriptions specify the relationship between neighboring “cells” in the pattern. In other words, the long range ordered materials formed by the miniblocks have a predictable pattern at the nanoscale to microscale, which persists over many repetitions of the nanoscale or microscale pattern to create long-range order.

Examples of the new miniblock polymers include modified biopolymers, such as modified native proteins or polysaccharides, or synthetic polymers, such as recombinant proteins (e.g., silk-like or keratin-like proteins), polypeptides, polyesters, polyamides (e.g., nylons), polyimides, polyimines, or copolymers thereof.

“Miniblock polypeptides” can have an amino acid sequence designed with repetitive and pseudo-repetitive motifs arranged in different domains (blocks) within the sequence. These polypeptides often contain self-fabricating miniblocks with a high (e.g., higher than 20%, 25%, 30%, 40% or more) glycine content, and repetitive solubilizing blocks, which are typically chemically different from the self-fabricating blocks. Different patterns of monomers in the simple repeated motifs used to construct domains will favor different helical conformations. In these miniblock polypeptides, the pattern of glycines and/or prolines is especially important depending on the sequence. Glycine pattern and imino acid content (proline and hydroxyproline) are important to hydrophobic-hydrophilic patterns. These polypeptides can also contain short functional motifs designed to incorporate turns, crosslinks, and ligand binding sequences in specific locations. Miniblock polypeptides that contain a self-fabricating block and two solubilizing blocks, can form a hairpin conformation, especially if the hetero-oligomeric self-fabricating block contains greater than 40% glycine and less than 10% imino acids such as proline and hydroxyproline. In other variants, different compositions and patterns of amino acids can be used to induce coiled-coil aggregates, or a conformation transition into an intrachain hydrogen bonded structure such as an α-helix. The miniblock polypeptides can also contain at least two self-fabricating blocks and at least two solubilizing blocks, wherein the self-fabricating blocks can be chemically the same or different from each other and the solubilizing blocks can be chemically the same or different from each other. In other embodiments, the new miniblock polymers can include amide linkages such as nylons and their copolymers. Accordingly, this invention relates to new mini-block polymers that are precisely designed so that they can undergo the above-described transitions.

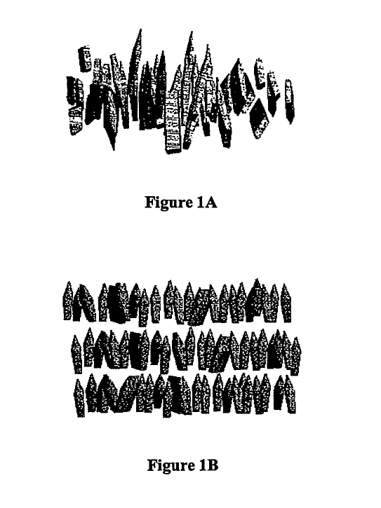

In another aspect, the invention relates to three-dimensionally patterned, self-fabricated materials having a long-range order, which are prepared from the new miniblock polymers through self-fabrication. These self-fabricated materials are usually in highly ordered liquid crystalline or liquid crystal-like phases, and can be in the form of films, fibers, and tapes. The materials are typically comprised of individual nano-scaled layers of miniblock polymers, in which the layers are often parallel to the plane of the material surface, but the layers also twist to form periodic domains with a different layer orientation. The miniblock polymers comprising the new materials are found in rigid conformations such as triple helix and β-strands in hydrogen bond stabilized β-hairpins formed by, e.g., triblock polymers (see, e.g., FIG. 1.).

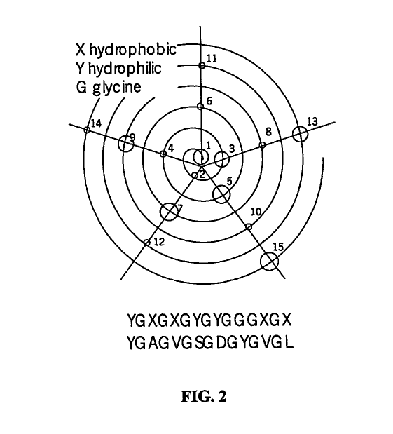

In the miniblock polypeptides, most of the conformations of the self-fabricating blocks can be defined by ranges of backbone torsional angles “phi and psi” (see, e.g., FIG. 2), where phi ranges from −20 to −160 degrees, and psi ranges from 90 to 180 degrees.

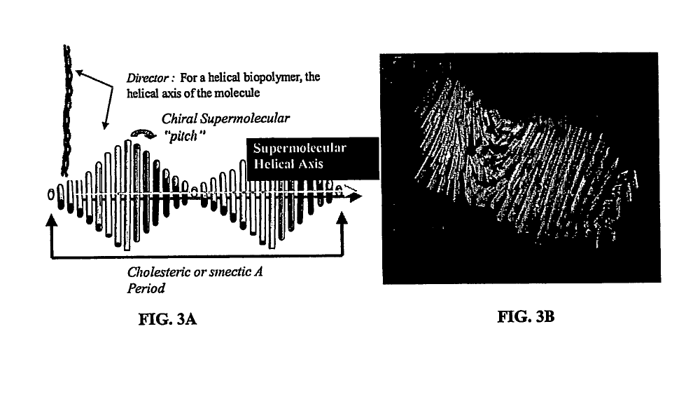

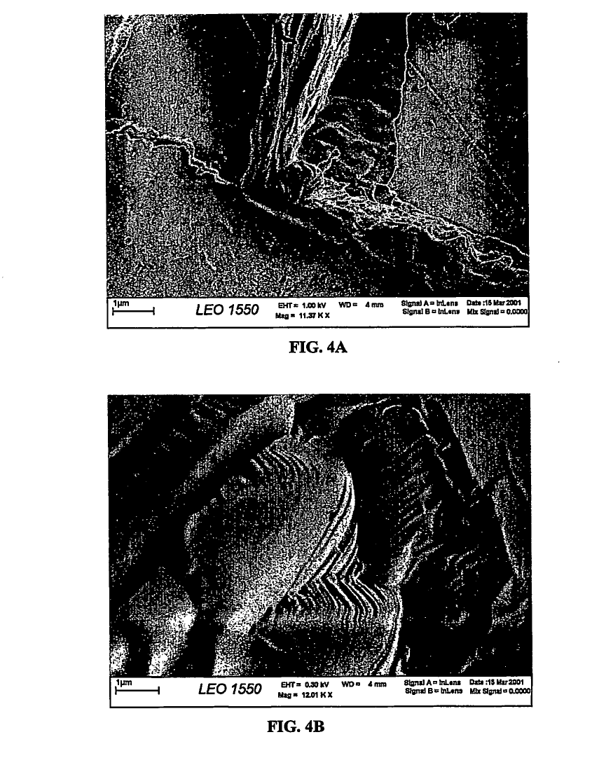

The self-fabricated materials do not have to be arranged in layers. In less ordered liquid crystalline phases (e.g., cholesteric, see FIG. 3), the liquid crystal will have a local “typical” orientation, which varies sinusoidally throughout the material. In materials made from less ordered liquid crystalline phases, such as the cholesteric phase, there will be a sinusoidal variation in chemistry at every free and every fracture surface, defined by the position and surface availability of the different blocks (see FIG. 4). In addition, arrangements of molecules are possible with local short-layered structures frequently broken up by periodic “defects” (derived from liquid crystalline twist-grain-boundary (TGB) phases), where defects are sharp sudden changes in layer orientation.

Also within the scope of the invention are methods of controlled delivery of drugs by incorporating one or more drugs within one of the self-fabricated materials and administering to a subject in need thereof an effective amount of the drug-containing material.

The invention also includes methods of modifying the optical response of devices in the near to mid infrared wavelength range (e.g., 1-20 micron wavelengths) by applying one of the self-fabricated materials to the surface of the device. Thus, this invention also relates to a method of enhancing IR absorption of a device by applying to the surface of the device a self-assembled material described above. The device can be an IR sensor or an IR filter.

Embodiments of the polymers include those having at least one of the helical segments comprising a peptide sequence having a glycine (G) residue at every third position, e.g., (G-V-P-G-P-P)ₙ; and those in which the non-helical segments are hydrophobic, in the polymer chains. The non-helical segments can contain glutamic acid.

Another aspect of this invention relates to a material made from a polymer through self-assembling in solution. By “self-assembling” or “self-assembly” is meant that “information” (e.g., the helical segments and amino acid sequence) within the polymer controls the combining of multiple polymers to form a unitary material. This combining of polymers occurs spontaneously or upon a triggering event, such as an environmental factor (e.g., temperature or solvent polarity).

Advantages of the miniblock polymers include that their structures, sequences (or building blocks), compositions, stereochemistry, orientation, spacing, and sizes can be readily and precisely controlled at the molecular and sub-molecular levels by changing the chemical composition of selected miniblocks, thereby tailoring properties (e.g., biological activities or mechanical properties) of these miniblock polymers. The miniblock polymers can be used as structural tissue implants, in liquid crystal displays, and for producing high-performance composites. The process that allows the miniblock polymers to self-fabricate into three-dimensional, self-fabricated materials can be selectively triggered by specific conditions and can be used in lieu of costly, error-prone, and time-consuming nanolithography steps, such as dip pen nanolithography in applications, such as fabricating chips, patterned surfaces, and nano-scaled materials that can be used as membranes, fibers, absorbents, reactive materials, catalysts, or catalyst-carriers and templates upon which to couple other reactants in ordered arrays.

The details of several embodiments of the invention are set forth in the description below. Other features, objects, and advantages of the invention will be apparent from the description and drawings, and the accompanying claims.

Definitions

For convenience, certain terms employed in the specification, examples, and appended claims are collected here.

The articles “a” and “an” are used herein to refer to one or to more than one (i.e. to at least one) of the grammatical object of the article. By way of example, “an element” means one element or more than one element.

A “block” is a region of a polymer composed primarily of (i) a single monomer type, (ii) a well-defined repeated motif, or (iii) a well-defined alternation of motifs (in which case the shorter alternating motifs can be linked together as a longer repetitive motif). A “miniblock” is a block that is not longer than about 230. Angstroms fully stretched (e.g., 40-200, 50-150, or 75-125 Angstroms), or a block that is comprised of no more than about 80 monomers (e.g., 3, 5, 10, 20, 30, 50, or 75 monomers). The terms “block” and “miniblock” are interchangeable in this application. Each miniblock consists of either a single type of monomer (and therefore is also called a “homoblock”), or two or more (e.g., 3, 4, 5, or more) chemically different types of monomers arranged in a pattern to form substructures (also referred to as “motifs”) repeated throughout the miniblock (and therefore is also called “heteroblock”). The molecular weights of the new miniblock polymers are usually in the range from about 1,000 to about 300,000. The new miniblock polymers can contain one or more of a variety of types of miniblocks arranged in various patterns.

The term “motif” or “patterned motif” refers to a short sequence of monomers (e.g., 3-15 monomers) that are repeated as an intact pattern with few (<20%) substitutions throughout a region of the sequence of the polymer or oligomer, and which adopt a well-defined regular structure. A motif generally serves a specific and well-defined binding function, folding function, or other function that is not common to other patterns of monomers of similar length. This will generally have a preferred secondary structure, solubilizing function, specific binding function or other feature to distinguish it.

A “solubilizing block” is a block that can cause a polymer to dissolve or form micelles in a medium such as an aqueous buffer or organic solvent. In principle, solubilizing blocks contain monomers that are ionic, charge-free, hydrophilic, or hydrophobic, depending on the nature of the solvent in which the new miniblock polymers are to be dissolved. Examples of such monomers include styrene, vinyl, pyrrolidinone, amino acid, acrylate, acrylic acid, ester, alkylene oxide, alkylene imine, and naphthylene.

A “conformation-forming block” (also called a “structural” “functional” or “self-fabricating” blocks) is a block that facilitates and controls the change in a polymer's super-secondary and secondary structure, thereby enabling the polymer to transform from a flexible amphiphile into a rigid, asymmetric molecule by undergoing a conformational change, e.g., folding, forming helixes, or transforming into a shape, such as a hairpin. Self-fabricating blocks can contain monomers such as amino acids, pyridine, phthalamide, and amides and are generally heteroblocks.

The term “motif template” or “motif pattern” refers to a short sequence containing monomer “native cards” used to describe families of specific motifs and to design and construct motifs. An example is a pattern containing glycine residues and hydrophobic and hydrophilic “native cards,” which are used to create a silk-like oligopeptide structural block (e.g., GXZGGZ, wherein X is a hydrophobic native card and Z is a hydrophilic native card). Another example is the GXZGXZ pattern where greater than 50% imino acids in the “XZ” positions are specified, as a generalized collagen-like motif or motif template. In very specific biological examples in the literature, these types of templates are referred to as “consensus sequences.”

The term “conformation” or “secondary structure” refers to a three-dimensional geometric structure or pattern formed when the monomers in a block adopt identical torsional angles or a well-defined and repetitive pattern of torsional angles. One example of a secondary structure for a structural block is a helix, which is a geometric structure resembling a screw. Each monomer in a helix can be related to the next covalently bonded monomer through rotation and translation, where rotation occurs about the same axis that defines the translation direction. Helices are often notated in terms of the symmetry (tightness) of the screw structure, where the number of rotation and translations required to complete a 360 degree turn define the screw. For example a 31 helix has three units separated by a translation and a rotation of 120 degrees. Another common notation simply specifies a number of covalently bonded chemical units (monomers) required to complete an integer number of full turns. For example a 5/2 helix requires 5 monomers to complete 2 turns (2.5 monomers complete one turn). A “designed helix” is a fully specified sequence of amino acids based on a well-defined motif or motifs arranged into blocks, where a selected motif template has been used to create a polymer that favors a particular conformation and has particular well-defined interactions with other helices.

The term “super-secondary structure” refers to a three-dimensional geometric structure formed by all or part of a polymer when a small number of secondary structures are combined in a well-defined manner. For example, a “Greek key” motif is a super-secondary structure comprised of a small number of beta strands (helical secondary structures) separated by turns.

A “coiled-coil” resembles a multistrand rope formed from several blocks in helical conformations deformed to wrap around each other. A coil is an aggregated structure and also represents a distinct conformation.

“Supercoiling” is a description of the deformation in the conformations of the individual strands in the rope needed to create the aggregate conformation. It is also a description of the aggregate conformation, usually referenced to the individual strands' non-aggregated conformation. For example, the collagen triple helix contains three strands, which typically form a closely related, un-aggregated helical structure called polyproline II. Formation of the triple helix requires a slight deformation in the conformation of each strand, i.e., they adopt a shorter, more compact helical structure when coiled together. The amount by which the triple helix is shorter is the “supercoiling.”

The term “folding” refers to a structural transition whereby the secondary structure in an entire polymer molecule is converted from an unrelated (or weakly related) sequence of helices into a structure where the monomers in a helix have well-defined “neighbors” which are in close spatial proximity, but not necessarily nearby in the primary sequence.

A “flexible amphiphile” is a molecule that has both hydrophobic and hydrophilic blocks. The blocks are sufficiently flexible to bend back upon themselves, to deform into micelles, and to adopt statistically defined, but non site-specific, interactions and three-dimensional structure. More generally, a flexible amphiphile is molecule with chemically distinct blocks in which a three-dimensional structure is not imposed by structural rigidity of any block, leaving chemical compatibility the dominant force to position the monomers.

A “lyotropic liquid crystal” is a phase formed by a flexible amphiphile (most general definition) with a supermolecular, well-defined geometry of regions rich in the different blocks. This supermolecular geometry forms a repetitive pattern that defines grains in the liquid (or solid) material. Molecular orientation is localized in the interfaces or boundaries between regions enriched in different monomer chemistries.

The term “long-range ordered surfactant phase” refers to the phase of a lyotropic liquid crystal formed by a surfactant where a persistent (translationally periodic) geometry of membranes or bilayers is established.

A “thermotropic liquid crystal” is a liquid of anisotropic and rigid molecules or well-defined supermolecular units, in which the anisotropy and rigidity dictate a packing of molecules that is not statistically uniform in all directions, i.e., an anisotropic liquid of anisotropic rigid constituents. Thermotropic liquid crystals typically possess local molecular orientation, which is not confined to a membrane or bilayer interface, but rather persists on a supermolecular scale due to the rigidity of the molecules and order or type of orientation is determined by temperature.

A “lyotropic rod-like liquid crystal” or “solvent intercalated liquid crystal” is an anisotropic liquid of rigid anisotropic molecules (or other well-defined units such as viruses or chitin crystals) where solvent is present in the liquid state—a liquid crystalline solution.

A “one-dimensional liquid crystal” refers to an anisotropic liquid where the local order in the liquid state is an average orientation of the anisotropic molecules along a unique axis.

A “nematic liquid crystal” refers to a one-dimensional liquid crystal in which local alignment and interactions between anisotropic units are uniaxial and there is no innate tendency to twist.

A “cholesteric liquid crystal” refers to a “one-dimensional” liquid crystal in which the local alignment is well defined, but the unique alignment axis tends to twist in a predictable manner as one moves through the material. Such a material is thus slightly biaxial.

A “smectic liquid crystal” refers to a liquid crystal in which the anisotropic molecules are oriented and are arranged in layers. The layered structure is well-defined and highly periodic perpendicular to the layer planes. The packing of the molecules within the layers resembles a liquid.

A “ferroelectric liquid crystal” refers to a smectic liquid crystal (molecules arranged in layers) in which the “up” and “down” directions for a molecule are not equally represented, resulting in a net dipole moment and effects such as piezoelectricity.

A “hexatic liquid crystal” refers to a smectic liquid crystal with limited packing order within the layers.

A “smectic structure” refers to a structure with layers and orientation representative of a smectic liquid crystal, but not necessarily in the liquid state, e.g., the structure of a material dried from intercalated smectic liquid crystals.

A “smectic layer” is a nanoscopic layer of aligned molecules in a smectic structure.

A “nanopatterned material” refers to a material with nanoscale features arranged in a predictable (translationally periodic) rather than statistical manner.

The term “long-range order” refers to the persistence of the structure and properties of a small material region across large length scales through translational periodicity. A pattern is formed by identical tightly packed “cells,” rather like tiles.

The term “material pattern wavelength” or “material periodicity” refers to the distance traversed in a material before returning to an area with the same local orientation. The definition of a wavelength in a material (or a periodicity) can depend on the length scale of the structures used to define orientation. For example, in a smectic material, layers may twist and molecules within layers may also twist or tilt. Thus, the distance traversed to recover an original layer orientation may not be the same as the distance needed to recover a molecular orientation. If these distances are not integer multiples of one another, a third “wavelength” or periodicity is defined as the distance required to recover the starting layer and molecule orientation.

The term “chirality” refers to handedness, which results in a tendency towards biaxial alignment and packing interactions (preferred tilting and twisting directions) of molecules and supermolecular structures in a material. It includes chemical chirality, which is the presence of an optically active chiral center (defined at a particular atomic location on the picoscale). However, less localized chiral interactions and effects are included as well. In the case of polymers, chirality can (and often does) refer to handedness of a conformation such as a helix, or even to the handedness of higher level (larger) ordered superstructures such as twisting layers in a material.

The term “phase transition” refers to the process of going from one arrangement of matter to another. This can be at the molecular level, e.g., folding, aggregation, or change in conformation; or at the material level, e.g., changes in liquid crystalline state or crystallization.

The term “twist-grain-boundary (TGB) phase” refers to a type of soft solid or liquid order, which is intermediate between cholesteric and smectic phases and involves very short-layered domains separated by a regular pattern of boundaries where the layers are twisted or tilted relative to their neighbors. A phase intermediate between different smectic phases where short domains of layered structure are separated by tilted or twisted boundaries.

The term “frustrated phase” or “frustrated morphology” refers to a state in which chemical complexity results in a worse thermodynamic state (i.e., higher free energy) for some portions of a molecule or material as an unavoidable consequence (not kinetic) of creating a better thermodynamic situation (i.e., lower free energy) for other portions of a molecule or material.

The term “nanomaterial” refers to a material having distinguishable features on the nanometer scale, typically one to several thousand nanometers.

The term “oriented nanomaterial” refers to a material in which distinguishable features at the nanoscale are oriented in a predictable manner relative to one another at the macroscopic (visible light) length scale.

The term “chemical patterning” refers to the creation of a geometric pattern of chemical entities or groups on a surface, or in a three-dimensional material.

A “thin film” refers to a film having a typical thickness of several microns to a millimeter, whereas an “ultrathin film” refers to a film having a thickness of less than a micron. A “thick film” refers to a film thick enough to have properties essentially the same as a bulk solid, but having the macroscopic form of a sheet.

A “bulk material” is a three-dimensional material or liquid, and not a thin film, interface, or coating.

The term “thermal stability” refers to the ability of a material to resist property changes in response to heat In the solid state, it can be reflected by the temperature at which distinguishing X-ray scattering features disappear or shift (thermal stability by X-ray), the temperature at which optical features and optical clarity are lost (thermal stability through optical measurements), or the temperature at which heat of melting is evolved (thermal stability by differential scanning calorimetry). In the liquid state, thermal stability can be indicated by the temperature at which distinguishing conformation signals are no longer detectable in a spectrometer (CD, FTIR). For instance, loss or change in spectral features signals protein denaturation.

The term “gel-like” refers to a highly viscous liquid state that retains solid form against gravity, but yields to slow imposed deformations. Local flow and rearrangement are observed within “gel-like” materials when swollen, but the overall shape is retained over timeframes of days to weeks. No chemical crosslinks, physical gelation, or network structure is implied.

The terms ortho, meta and para apply to 1,2-, 1,3- and 1,4-disubstituted benzenes, respectively. For example, the names 1,2-dimethylbenzene and ortho-dimethylbenzene are synonymous. Further, the abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

It will be understood that “substitution” or “substituted with” includes the implicit proviso that such substitution is in accordance with permitted valence of the substituted atom and the substituent, and that the substitution results in a stable compound, e.g., which does not spontaneously undergo undesired transformation, such as by rearrangement, cyclization, elimination, etc. Further, as used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. All such isomers, as well as mixtures thereof, are intended to be included in this invention.

For purposes of this invention, the chemical elements are identified in accordance with the Periodic Table of the Elements, CAS version, Handbook of Chemistry and Physics, 67th Ed., 1986-87, inside cover.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Mini-Block Polymers

The present invention provides miniblock polymers with a block structure based on block architecture templates as well as designed modifications to existing polymer architectures. Specifically, these miniblock polymers include chemically distinct hetero miniblocks, which are themselves copolymers (e.g., alternating copolymers and terpolymers). Some of the miniblocks are very short (e.g., consisting of 3 monomers) and are able to impart to the polymers specific chemical, biological, and physical functionality, such as cell surface interaction sites, cross-linking sites, optically active sites, and metal-binding sites. The heterogeneous nature within each block allows variations in the monomer sequence and function of the polymers, as well as the opportunities for incorporating useful moieties such as ligand binding epitopes within a core or end block. In addition, the low to moderate total molecular weight and block size favor smectic-like and liquid crystalline driven types of organization rather than microphase separated (unoriented) nanodomains occurring in high molecular weight synthetic block copolymers. The moderate size of the rigid units in the folded conformations also favor self-assembly in a reasonable time scale, with a relatively low occurrence of errors, mismatched orientations, and domain walls and boundaries as compared to a polymeric liquid crystal. The heterogeneous chemical nature incorporated in the blocks and consequent high solubility of the selected regions results in enhanced transport in a concentrated solution, where molecules will be expected to diffuse preferentially in a linear fashion and the soluble regions can “lead” diffusion modes such as reptation and aid in cooperative absorption from the bulk.

The miniblock polymers have precise molecular weights, miniblock sizes, and chemical structure controls to enable users to precisely manipulate and position the physical and chemical processes of a long-range ordered material self-fabricated from the miniblock polymers to an accuracy of 2 nm.

In addition to highly engineerable designed miniblock polymers, the invention also provides nanostructured, self-fabricated materials formed from these miniblock polymers. The design of the polymers dictates the features of the self-fabricated materials, such as nanopatterned long-range ordered solids. These features include processes that work with the thermodynamic evolution of long-range ordered phases to create extremely stable nanostructures.

For instance, amphiphilic miniblock polymers can be used to create lyotropic liquid crystalline order as a precursor phase. Formation of large domains of highly oriented liquid crystalline structures (or thermotropic liquid crystals with solvent, or lyotropic rods, or “solvent intercalated liquid crystal”) can be achieved through folding to form a rigid amphiphilic structure from an ordered flexible amphiphilic precursor phase. A controlled folding transition can be used to control how diffusion and fluidity/solidity develop while a solvent intercalated liquid crystalline phase is formed, thus allowing control of grain size, structure, and pattern. A controlled transition to a more rigid (slower moving) structure can “freeze in” liquid crystalline features in the solid state. Further, the design of multiple potential transitions or nanochemical and nanophysical “events” into the molecule such that many of the individual “events” respond most strongly to different physical and chemical “triggers” or stimuli and can thus be manipulated in a somewhat separate manner.

The self-fabricated materials obtained from the miniblock polymers use the complex molecular design of the miniblocks to incorporate chemically (e.g., an acid or metal binding site), physically (e.g., an optically active site), or biologically active groups (e.g., cell recognition domains or molecule-molecule interaction sites), which are spaced out at a well-known distance, and oriented by the self-fabricating attached blocks, but are arranged statistically throughout the material rather than in highly ordered layered structures. These materials will have sinusoidally varying chemical properties (availability and surface “exposure” or the physically, chemically, and biologically active groups) at every free and fracture surface in the material, e.g., as shown in FIG. 4.

The self-fabricated materials, which have a multi-layered flat nanolayered structure, can also be composed of miniblock polymers, in which physically, chemically, and biologically active groups are arranged and oriented in “sublayer” regions, as shown in FIG. 7. In other words, the self-fabricated materials can have a complex structure such as a hierarchy of twisted, well-defined aggregates of layers and exhibit clustering of regions of parallel lines with a high surface area of exposed chemically active groups.

Further, the self-fabricating materials can be composed of miniblock polymers having a self-fabricated microscale (e.g., 1-10 microns, triple helix self-fabricating block) or nanoscale (e.g., 300 nanometers, β-hairpin self-fabricating block) grid or mesh pattern on their exposed surfaces, where the mesh is well defined, has a simple symmetry (e.g., tetragonal, cubic, or hexagonal), and areas chemically different from the background material. Such miniblocks polymers can also self-fabricate under mild chemical conditions to give materials that exhibit irreversible or stable differences in solubility, structure, and other properties with respect to unordered molecular solutions and solid glasses.

The self-fabricated materials have significant advantages over traditional materials. First, they eliminate costly, error prone, and time-consuming nanolithography steps such as dip pen nanolithography. Second, the chemistry of the surface pattern is determined by groups or blocks covalently bound into the miniblock polymers in a separate step. The types of alternating chemical patterns are not constrained by coupling chemistries available through adsorption. Third, the pattern size is determined at the molecular structure level and is self-assembled into a semisolid state. Spreading, poor wetting, etc. are no longer issues in creating lines, and very small stripes (size scales which represent subportions of the molecules used) and patterns can be created. Complex patterns of alternating stripes can be created with controlled size and spacing. Fourth, the orientation of molecules is also controlled, resulting in a chemical pattern in which the chemistry is known down to the molecular and submolecular scale. Fifth, the patterned bulk materials are three-dimensional.

Key features of the self-fabricated materials obtained from the miniblock polymers, include liquid crystalline order analogous to oriented thermotropic liquid crystals, large domain sizes, and the ability to retain these features in the solid state. Chemical complexity is designed into the miniblocks to enable one to engineer frustrated structures. A large degree of physical, chemical, and biological functionality is “built in” to the monomer sequence of the miniblocks, resulting in precisely located interaction sites and in addressable shape changes. This is especially true when the self-fabricated materials are induced to form nanolayered structures. For example, a miniblock oligomer with a “B” block—“A” block—“B” block structure is said to have a “triblock” structure as shown in FIG. 1. When this type of miniblock oligomer forms highly oriented layers, each one molecule thick, the chemistry (pattern of miniblocks) in the oligomer defines patterned properties in the material. The triblock structure and layered arrangement, taken together, result in sublayers of solubilizing blocks. The solubilizing blocks in this minitriblock structure are on the ends of the polymer (on each end of the self-fabricating block). Thus, the solubilizing block sublayer contains many dangling polymer chain ends, and can be easily swollen with other molecules (such as metals, catalytically active molecules, magnetic ions and small nanoclusters, optically active molecules). The length of the (gray) self-fabricating block defines the distance between the solubilizing block regions, and hence the distance between “lines” or “planes” in a pattern of highly functionalizable regions. This distance can be defined at the sequence design level by selecting the repetitive motif for a particular conformation and repeating it a certain number of times to form a self-fabricating block. Each conformation (provided that it consists of a repetitive pattern of torsional angle values for the sequence of monomers, defining a helix) has a particular vertical distance (along the long axis of the helix) associated with a monomer. In other words, to go from one monomer to its neighbor in a defined helical conformation, one goes “up” a set distance, which is known. Thus, very precise length and layer thickness control can be obtained by selecting known conformations (e.g., by selection of a motif or patterned motif template) and the number of monomers included to make a self-fabricating block.

The molecular weights of the miniblock polymers are usually in the range from about 1,000 to about 300,000. An increase in the molecular weight increases the range of temperature at which self-fabrication of the new miniblock polymers occurs. Increases in molecular weight allow the polymers to be increasingly chemically complex, and high molecular weight polymers are useful in the formation and stabilization of more complex patterns of material orientation and chemistry. Features such as entanglements at high molecular weight impart elasticity and structural stability to a material at points where a low molecular weight polymer would have softer local domain structures. Local stress caused by the interactions of different segments in a high molecular weight polymer can be propagated through larger distances along the (longer) chain, and are useful in producing conformation changes in attached blocks. Thus, a high molecular weight miniblock polymer can have self-assembling “A” blocks and fabricating blocks which are not in close proximity, yet which interact and react to changes elsewhere in the polymer.

Complete structural control in a biopolymer based material, or a bioinspired or biomimetic synthetic polymer material, is achieved through design of a patterned miniblock polymers comprised of subdomains or “miniblocks.” The different blocks can be homo-oligomers, comprised of a single monomer repeated to form a block. However, the different blocks are more typically hetero-oligomers made up of a simple pattern of monomers that repeats to form the block. Some deviation from strict repetition is tolerated without losing block function (typically <10% variation in pattern repetition). Different types of blocks can be defined in terms of their function as well as the typical monomer patterns, numbers of repetitions, and chemical nature describing the block composition. In addition, some very short functional patterns are included and described as “blocks” even though they are rarely repeated. These patterns (described throughout the document as “C” and “D” blocks) impart significant controlled functionality to the designed polymers and oligomers, and are included in the descriptions of “blocks” to simplify descriptions of overall polymer and oligomer design.

A key feature distinguishing the design of these miniblock polymers from synthetic block copolymers is the ability to design specific and complex functions into the various blocks through use of multi-monomer patterned motifs. Synthetic block copolymers utilize simple chemical segregation to achieve long-range ordered patterns. The oligomers and polymers described herein possess the additional ability to change their structure and associated chemical and physical properties (analogous to the manner in which a protein folds). A key feature distinguishing these polymers from existing approaches to protein design is the incorporation of blocks whose “function” is to form long range ordered predictable structures. The design and incorporation of these structural blocks constitutes a major aspect of the invention.

The structural blocks are also called “self-fabricating blocks,” because they are the blocks that undergo conformational changes and form well-defined geometric molecular structures for self-fabrication into three-dimensional materials. In polypeptides, the fabricating blocks can be rich in glycine and are able to cause folding or formation of helices within the polymers. When the fabricating blocks are folding blocks, the linear polypeptide chain structures serve two functions. First, they can fold and form tightly folded molecular conformations or pseudo-tertiary structures, which are stabilized by hydrogen bonds within the polymer blocks comprising each chemically disparate domain, and will form distinct rigid shapes. Second, the folded polypeptide molecules can form micelles or vesicles or pack asymmetrically, through aggregation in an oriented or ordered manner in a solution at a sufficiently high concentration (e.g., saturated) and a low temperature (e.g., 0 or 1° C.). The presence of the folding transition aids in transport of the polypeptide molecules and facilitates their self-fabrication into large, ordered, semi-solid materials. The materials have a mechanically stable chiral long-range ordered polymer structure (at the material level, or nanometer to millimeter length scale).

Design of Miniblock Polymers

Miniblock polymers can be made of a wide variety of monomers, including amino acids to form polypeptides, diacids and diamines to form polyamides, and diacids and diols and diamines to form poly(amide-co-ester)s. The miniblock polypeptides can include “A,” “B,” “C,” and “D” blocks, which can be either peptidyl blocks or non-peptidyl blocks and are described below in detail.

Miniblock polymers in the form of polypeptides can be designed such that they are mainly (e.g., >80%) comprised of two different types of blocks, i.e., solubilizing blocks and self-fabricating blocks, arranged in fairly short (e.g., 3-12 amino acids for solubilizing blocks, and 12-60 amino acids for self-fabricating blocks) linear domains. Useful size ranges for the self-fabricating blocks are 12-36 monomers and useful sizes for the solubilizing blocks are 3-8 monomers. However, some self-fabricating blocks, such as turns and binding sites, are typically very short, e.g. from 3 to 10 monomers. Typically the self-fabricating (e.g., “folding” or “associating” or “ordering”) blocks will comprise more than 70% of the linear structure. The large proportion of self-fabricating blocks causes the thermodynamic behavior of the miniblock polypeptides to be dominated and driven by the self-fabricating blocks. As a result, the self-fabricated and solubilizing blocks are “carried along” and can be stably patterned and placed in geometric patterns which they would not normally adopt. By designing a complex multiblock polymer with hetero oligomeric motifs incorporated in the self-fabricating block, one can use thermodynamic frustration to create nanoscale patterns that persist as long-range ordered textures. The designed use of thermodynamic frustration is a key feature of material fabrication from miniblock oligomers and polymers.

In the polypeptide sequences described herein, the following formats and rules apply. In a series of amino acids separated by commas and enclosed in square brackets as in [M, N, P], it is understood that the amino acid “M,” “N,” or “P” can occupy that position in the sequence interchangeably. A sequence in parentheses followed by a subscripted number as in (X₁X₂X₃X₄X₅)ₙ is a repetitive motif or submotif (a well-defined component of a block), which is repeated “n” times within the larger block. Here “X” represents any amino acid. The letters “X” and “Z” are used throughout the document as “native cards”. When only “X” is specified “X” can be any amino acid. When “X” and a specific amino acid are used “X” is understood to represent any amino acid other than the one specified. When both “X” and “Z” are used, one is a hydrophobic native card (X) and the other is a hydrophilic native card (Z). When “X” is repeated in the description of a block or within a repeated sequence as in (GXGXX)ₙ, it is understood that the “X” residues are not necessarily identical in the block within parentheses, and the identities of the “X” residues are not necessarily preserved throughout the “n” repeats of the residue. It is further understood that the general overall blocks described can be repeated end to end to attain a desired molecular weight in the block, or can be combined either end to end or with an intervening amino acid to create an “offset” in the glycine pattern of appended blocks.

“A” Blocks

The so-called “A” blocks are self-fabricating blocks in the miniblock polymers. In polypeptides, “A” blocks can be any of four different types shown in the following table:

Self-Fabricating Blocks or “A” Blocks

| | | | 3. Folding or | |
|---|---|---|---|---|
| Structural | 1. Aggregation and | 2. Secondary | super-secondary | 4. Conformation |
| transition | conformation | structure | structure | and folding into |
| type: | transition | transition | transition | paracrystal |
| Biological | Collagen, keratin | α-helical | Spider silk, | Amyloids, |
| examples | coiled coils | proteins, elastin | silkworm silk | cross-β-sheet |
| | | | | silks |
| Process | Temperature - | Solvent polarity - | Concentration - | Concentration - |
| parameters | strong | strong | strong | Strong |
| used to induce | Solvent polarity - | Temperature - | Solvent - | anticorrelation |
| and address | moderate | moderate | moderate | Solvent - strong |
| transition | Concentration - | Concentration - | Temperature - | Temperature - |
| | weak | weak | weak | moderate |
| Key | High content of | 5-mer or 4-mer | High glycine | Motif with a |
| composition | imino acids | motif | content with an | strongly |
| features of | (proline and | incorporating | alternating | heterogeneous |
| biomimetic | hydroxyproline) >25%; | polar-polar or | motif - GGX or | steric and |
| examples | Glycine | acid-base bridges | GX; often a | chemical pattern |
| | every third position | between 1^{st} and | hydrophobic | resulting in |
| | in strict alternation | 5^{th} (4^{th} in 4-mer) | nucleating | regular turns. |
| | | monomers | sequence (A, V, | Often >40% |
| | | | L, I, Q in groups | glycine or very |
| | | | of 4 or more) | hydrophobic |
| General | Steric hindrance | Side chains | High glycine | Hydrophobic |
| chemical | periodically | which “fit” into | content, small | nucleation motif |
| features of | incorporated into | α-helix, 4-mer or | changes in non- | surrounded by |
| biomimetic | sequence - ex: | 5-mer pattern | glycine | turns consisting |
| examples | rings bridging | with hydrophilic | components | of hydrophilic, |
| | different polymer | 1^{st} first and last, | define more and | glycine, and |
| | backbone sites | other monomers | less hydrophilic | large monomers |
| | | hydrophobic | “blocks” | |
| Restrictions | Shorter sequences | At least one full | Repetitive | >40% |
| on size, | aggregate at lower | repeat of a 5-mer | sequence at | hydrophobic |
| chemistry, | temperatures. | motif, 2 full | least 12 | monomers |
| and | Hydrophilic | repeats of a 4- | monomers long, | |
| composition | directly after | mer required to | tolerates a range | |
| | glycine lowers | stabilize | of chemical | |
| | stability, directly | structure | compositions | |
| | before glycine | | | |
| | enhances stability | | | |
| Structural | Aggregation and | Secondary | Folding or | Conformation |
| transition | conformation | Structure | Supersecondary | and folding into |
| type: | transition | Transition | Structure | paracrystal |
| | | | Transition | |
| Biological | Collagen, keratin | α-helical | Spider silk, | Amyloids, |
| examples | coiled coils | proteins, elastin | silkworm silk | cross-β-sheet |
| | | | | silks |
| Major | FTIR - amide A | CD spectroscopy | Solubility | Solubility |
| techniques to | X-ray diffraction | FTIR | FTIR | FTIR |
| characterize | Ultracentrifugation | Electrical | Electron | Electron |
| transition | Chromatography | properties | microscopy | Microscopy |
| | Light scattering | measurements | X-ray | X-ray |
| | | | diffraction | diffraction |
| Dominant | Smooth chiral | Multilayers with | Textured thin | Fibrils and |
| long range | multilayers, small | low chirality and | films and self- | complex fibers |
| ordered | variations in layer | alternating up- | assembled | |
| material form | orientation | down orientation | “tapes” | |

Type 1 “A” blocks are analogous to collagen triple helical structures and undergo a conformation and association or aggregation transition to form rigid structures. Type 2 “A” blocks are similar to keratins and other α-helical proteins and polypeptides. These blocks form rigid well-defined structures through a conformation transition from random coil to a well-defined hydrogen bond-stabilized helix. Type 3 “A” blocks are similar to insect silks and have been engineered to undergo a simple folding transition, forming rigid hydrogen bonded “hairpin” structures. Type 4 “A” blocks are similar to prions, amyloids, and some beetle silks, and undergo a folding and paracrystalline transition to form rigid “self-crystallized” structures. Different sequence patterns are required to allow the different types of transitions that define “A” blocks types 1-4. These sequence patterns can be generalized so that many patterns can be described using a simple “sequence template” containing a pattern of specified amino acids and “native cards.” The templates for each type can in turn be further generalized in terms of the criteria used to generate them. Some sequences that satisfy these criteria occur naturally, or are variants or derivatives of naturally occurring proteins or protein fragments.

In the following sections the different types of “A” blocks are described in more detail for polypeptides in terms of their most general design criteria, followed by a set of typical examples or sequence templates, followed by a set of fully specified “A” block sequence examples. A similar “general to specific” approach is used to describe B, C, and D blocks, although the lack of a repeated motif in these latter block types makes definition of template sequence motifs irrelevant in some cases.

Type 1 “A” blocks (structural blocks which aggregate along with a conformation transition) must contain glycine every third monomer, contain greater than 25% amino acids and have a total block length of more than 6 monomers. Type 2 “A” blocks are structural blocks which undergo a random coil to helix transition. Types 3 or 4 “A” blocks have two criteria:

Criterion 1—They Must Contain Glycine

Examples of glycine blocks include (GX)ₙ, (GGX)ₙ, (XG)ₙ, (XGG)ₙ, (GXX)ₙ, (XXG)ₙ,(GGXGXX)ₙ (SEQ ID NO:2), (GXGXGGXGXX)ₙ (SEQ ID NO:3), and their combinations. Here “x” is a non-glycine native card. The combined blocks can be either directly connected as in (GX)ₙ(GGX)ₙ (SEQ ID NO:4) or separated by an intervening amino acid as in (GXX)ₙA(XXG)ₙ (SEQ ID NO:5) or (GGX)ₙG(GGXGXX)ₙ (SEQ ID NO:6).

Criterion 2—They Must be Conformation-Selecting Blocks or Derived from Native Sequences

Conformation-selecting blocks, also called “chemical blocks,” impart to overall miniblock polymer chains hydrophobicity or hydrophilicity that would favor and tend to stabilize secondary structure intermediates between a twofold (2/1) extended helix (or β-strand or helix with a 2₁ screw axis) and a threefold (3/1) extended helix (or polyglycine II structure with a 3₁ screw axis). Examples are the 12/5, 5/2, 8/3, and 11/6 helices. In this notation the numerator specifies the number of amino acids making the number of full circuits shown in the denominator. Thus, in a 12/5 helix, 12 amino acids are required to complete 5 full circuits or turns of the helix, or 2.4 amino acids per turn. The helical notation used for proteins does not distinguish between different conformations with the same number of residues per turn and different torsional angles.

Blocks derived from native sequences that meet criteria 1 and 2, either exactly or approximately, can be used in lieu of sequences synthesized according to the two criteria described herein. Many such sequence patterns occur in insect silks, chorion, amyloidogenic, and other related proteins. Examples of these blocks include those derived from an approximation of sequences found in native type B. mori silk fibroin, but are not identical to the native type sequences, such as GAGAGS (SEQ ID NO:7) and GAGAGY (SEQ ID NO:8); native type derivatives from silkworm silks, and core blocks with the form (G[A,V,L,I]G[A,V,L,I])ₙG[Y,S,T]) (SEQ ID NO:9) in which n ranges from 1 to 5 such that the core block is between 12 and 60 amino acids. Alternatively, one can mimic or approximate native protein sequences (such as those found in spider silks, prions, or amyloids) through substitution of the “X” amino acids in one of the glycine blocks described above in criterion 1 with native type sequence amino acids at the equivalent positions in the sequences.

Designing an “A” Block with an Un-Natural Helix Conformation

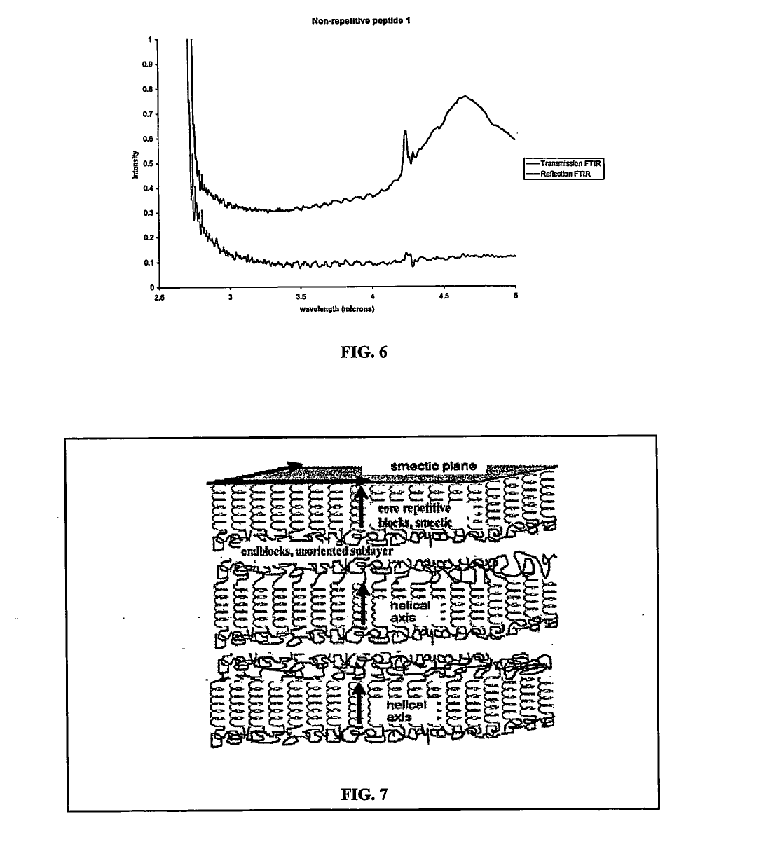

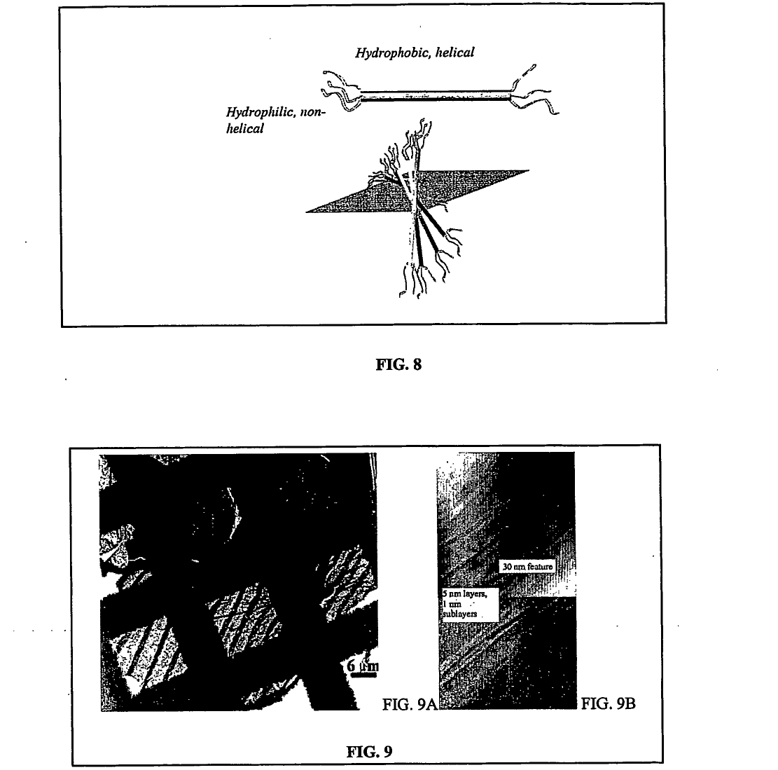

An example of conformation selecting block design is shown in FIG. 6. Schematics like the one in FIG. 6 can be used to illustrate how conformation selecting blocks are designed.

Step 1: One can draw a five-fold pattern of spokes over the helical spiral to allow easy placement and visualization of the 5 amino acids in 2 full turns of the helix. The number of spokes is the numerator in the “5/2” helix. Because two full turns are completed, when circles are placed along the helical spiral they sit on every second spoke rather than every spoke. For an 8/3 helix, one can have 8 spokes and a circle (representing an amino acid) following the helical spiral on every third spoke.

Step 2: A template motif consisting of a pattern of glycine and larger amino acids is overlayed. These templates are typically derived from native protein motifs. Glycine is specified and the positions of glycine is similar (>80%) to the native sequence used to derive the pattern because glycine is extremely small and achiral and can be used as a spacer in designed helix construction. The circles representing glycine are thus made smaller in order to keep track of the glycine pattern.

Step 3: Next we assign hydrophobic and hydrophilic amino acids to the large unspecified circles remaining. For most designed helices, assigning hydrophobic amino acids to one side and hydrophilic amino acids to the other is used to stabilize the desired conformation in helix-helix interactions (hydrophobic and hydrophilic helix sides match up), at an air-water interface, at the outer surface of a precipitate, and in other asymmetric environments. Often the hydrophilic and hydrophobic interactions are “tapered”, in other words more strongly hydrophobic and hydrophilic amino acids are selected for positions well away from the hydrophobic hydrophilic boundary and less hydrophobic and hydrophilic acids sit nearer the boundary. For complex sequences, gradations of colored shading in the large circles are used to represent variations in chemical properties.

Step 4: The patterned sequence motif template for the amino acid sequence design is read from the diagram by following the path of the helical spiral. For simple sequences the result is a pattern of letters representing glycine, hydrophilic, and hydrophobic locations (as above). For more complex sequences with graded chemistry a sequence of glycines and color-coded circles results.

Step 5: Appropriate amino acids are specified in the non-glycine positions to get a sequence motif which can be repeated to form a block (as above). Provided that the hydrophilicity and hydrophobicity conditions are met, amino acids can be selected for specific interactions or the pattern a short sequence into the helix for cell-specific, inorganic specific, and other types of interactions. Most motifs tolerate some variation in pattern which allows us to functionalize them at the sequence level, for example by writing in an integrin-binding Arg-Gly-Asp (RGD) sequence, a crosslinking pattern of cysteines, or a multi-amino acid phosphorylation site. Furthermore, the basic patterned motif template can be used to generate families of similar helices either through explicit specification of different amino acids, or through shuffling experiments which randomly select hydrophobic and hydrophilic amino acids at the appropriate locations.

In general, regularly repeated patterns of monomers will result in helices. If the monomers are torsionally constrained (as in monomers containing aromatic rings) or if they interact with the other monomers in a predictable fashion (as in the series of salt bridges and hydrogen bonds in an α-helix), the helix formed will be rigid and stable. These general conditions are not limited to proteins and oligopeptides. Any chiral monomer or repetitive pattern of chiral monomers can be used to create a helix forming sequence in principle. Associated helices are known for a number of synthetic polymers and no protein biopolymers (for example dextran) and the chiral oligomeric forms of those polymers can be used to form “A” blocks.

Combining a glycine block with a conformation-selecting block by overlaying these two blocks would result in an “A” block sequence. FIG. 2 shows an “A” block containing both hydrophilic glycine patterns (G-Z) and hydrophobic glycine patterns (G-X). Such a block can form a helical conformation. Variations of naturally occurring sequences that satisfy the criteria can also be used. Shown below are examples of useful “A” blocks:

| | |
|---|---|
| GGAGGGGTGGLGSGGAGAGGLGGGGAG | (SEQ ID NO:10) |
| GDVGGAGATGGS | (SEQ ID NO:11) |
| GNVGGAGASGGS | (SEQ ID NO:12) |
| GAIGGVGATGGS | (SEQ ID NO:13) |
| SGAGVGRGDGSGVGLGSGNG | (SEQ ID NO:14) |
| GPGGTGPGQQGPGGTW | (SEQ ID NO:15) |
| GPGNNGPGGYGPGNNGPSGPGSA | (SEQ ID NO:16) |
| DPGVYGPSGNAPGVYGPSGQGAGAGS | (SEQ ID NO:17) |
| GVGVGS | (SEQ ID NO:18) |
| GIGIGS | (SEQ ID NO:19) |
| GVGGGY | (SEQ ID NO:20) |
| GAGAGY | (SEQ ID NO:21) |
| GAGAGD | (SEQ ID NO:22) |
| SGRGGLGGQGAGGGAGQGGYGGLGSQG | (SEQ ID NO:23) |
| MKHMAGAAGAVVGGLGGYMLGSAM | (SEQ ID NO:24) |
| GAGAGT | (SEQ ID NO:25) |

“A” blocks can also be those polypeptide sequences containing glycine in every third position. Examples of such sequences are GVPGPV (SEQ ID NO:26), GVPGPA (SEQ ID NO:27), GLOGPP (SEQ ID NO:28), GIPGPP (SEQ ID NO:29), GPPGPPGAP (SEQ ID NO:30), GAPGPPGAP (SEQ ID NO:31), and their combinations. Such “A” blocks cause formation of helices in the polypeptide domains.

“B” Blocks

“B” blocks are the “solubilizing blocks,” and are used to aid in the dissolution of the miniblock polymers and the formation of micelles of these miniblock polymers, and therefore facilitate the self-fabrication process as the molecular weights of the miniblock polymers increase. “B” blocks can be either primarily hydrophilic or hydrophobic, depending on the solvents. In polypeptides, “B” blocks are generally not rich in glycine. Examples of hydrophilic blocks include poly- or oligo-Asp, Lys, Asn, Ser, Tyr, Thr, Arg, and combinations of these amino acids. The hydrophilic “B” blocks can be all polar, all basic, all acidic, basic and polar, or acidic and polar, but not including acid and base combinations in the same block. Examples of hydrophobic “B” blocks include poly- or oligo-Ala, Val, Leu, Ile, Phe, and combinations of these amino acids. For non-peptide polymer blocks, examples of hydrophilic “B” blocks include polylactic acid, and polyvinyl alcohol; and examples of hydrophobic “B” blocks include nylon 66, polypyrrole, and polyoxymethylene.

“C” Blocks

“C” blocks are short sequences that impart special properties or functionalities to the miniblock polymers, as well as to materials fabricated from these miniblock polymers. Examples of “C” blocks include RGD, Met trigger, (SP)n flexible block, phosphorylation sites, and ligand binding epitopes. Triggers, such as oxidation-reduction or phosphorylation-dephosphorylation triggers can also be incorporated into the miniblock polymers. Such triggers allow one to control the initiation of self-fabrication by external or environmental conditions such as temperature, light, redox chemistry, enzymatic reactions, and pH. Examples of these triggers include acryloyl-L-proline methyl ester and N-isopropylacrylamide for temperature control, ethylene terephthalate for light activation, methionine and serine for redox chemistry activation, and N-isopropylacrylamide-co-acrylic acid and acrylamide-co-maleic acid for pH control.

“D” Blocks

“D” blocks can be short sequences of 2-5 amino acids or monomers. Examples of “D” blocks include GPG, which function as a “turn,” and links helical segments pointing in different directions in overall longer sequences, and GEG which also a turn sequence. The function in general of “D” blocks (as distinct from “C” blocks) is to fine tune and modify the geometry of the helical and folded structures adopted by the self-fabricating “A” blocks. For example, in a minitriblock oligomer with a hydrophobic silk-derived “A” block and acidic solubilizing “B” end blocks, a hairpin structure is adopted at high (>50 mg/mL) concentration or low pH (<3). The conditions at which the hairpin structure is formed and stable can be extended by putting a GPG “D” block turn in the middle of the “A” block sequence (the oligomer block structure changes from BAB to BADAB). If the D block interrupts the “A” block sequence(s) asymmetrically, in other words if the two resulting new “A” blocks are not the same length, an asymmetric hairpin can be forced. An asymmetric hairpin structure would be expected to crystallize far less readily than a symmetric structure. Thus this would be a useful modification to incorporate if excessive crystallization (instead of liquid crystalline long range order) was observed and was not desired.

Synthesis of Miniblock Polymers

The miniblock polymers can be prepared by methods known in the art. For instance, a miniblock polypeptide with a designed sequence can be prepared by solid-phase synthesis (see, e.g., Merrifield, Pure Appl. Chem., 1978, 50:643; Merrifiled, Angew. Chem. Int. Ed. Engl., 1985, 24:799; and Kent, Ann. Rev. Biochem., 1988, 57:957) or by recombinant synthesis with artificial genes (see, e.g., Krejchi et al., Science, 1994, 265:1427; and Evans et al., Science, 1996, 273:933), while a polyester containing, e.g., cholesterol repeating units, can be prepared by a condensation reaction between cholesterol and an acid under suitable conditions. The structure, sequence, size, composition, and stereochemistry of the polymers can be precisely controlled in these methods. It is the control of these parameters that provides the new miniblock polymers.

More specifically, to synthesize a miniblock polypeptide, gene shuffling or similar strategies can be developed by genetic mixing, followed by selection for a specific pattern or function. This approach expands the data sets and block designs that can be incorporated into the polypeptides to achieve specific functional outputs. The methods involve synthesis of oligonucleotides encoding selective blocks but with randomness in specific sequences, then using primerless PCR methods as described by Cho et al. (Appl. Envir. Microbiol., 2002, 68 (4): 2026-2030) to evolve the desired sequences. Designed sequences based on the criteria listed above would form the starting points for gene shuffling (in addition to being used as synthetic polypeptides). The use of designed patterns to “seed” a gene shuffling experiment will limit the number of sequences explored and help target useful properties.

Side chains of the “A,” “B,” “C,” and “D” blocks of the polymers provide opportunities for chemical modifications to the polymers, which in turn can control liquid crystalline behaviors of the miniblock polymers, thereby controlling the process of self-fabrication. Chemical modification of the side chains can also change the hydrophilicity or hydrophobicity of the miniblock polymers. Thus, one can tailor the geometrical form of the miniblock polymers and further fine-tune the self-fabrication mechanism. The differences in chemical disparities (e.g., hydrophobicity and solvent preference/solubility parameter) between the solubilizing blocks and fabricating blocks can be used to tune the self-fabrication pathway.

The miniblock polypeptide polymers can be prepared by combining the “A,” “B,” “C,” and “D” blocks described above, e.g., by coupling (e.g., covalently) the amino acid monomers (such as lysine, glutamic acid, and cysteine). See, e.g., McCarthy et al., J. Bio. Mat. Res., 2001, 54, 139-148. Examples of these new miniblock polymers include:

| | | | | | |
|---|---|---|---|---|---|
| ABA | BAB | AB | BA | CBA | CAB ABC |
| CB | BCA | BAC | ABD | DBA | ADB BAD |
| DAB | BDA | BCBA | ABCB | ADA | ABDB BDBA |
| ABDBA | ABDB | BDBA | BADAB | BADA | BCBDA |
| ADBCB | BDB | BDAB | BDACB | CBABC | BCBDADBCB |
| ABCBA | BCAC | CABAC | ACBCA | BACAB | CABDB |
| BCADB | CBADB | BADB | BDBAC | BDBACABDB | |

and any combinations and repetitions of the above. Molecules without “A” blocks will not self-fabricate through chiral rigid liquid crystals, but can form other ordered structures such as lyotropic surfactant phases, which can be solidified as well (due to the relatively large molar mass of the oligomers when compared to typical surfactants).

When a block type appears more than once in a patterned combination, for example ABA, this is not meant to denote that all the A blocks are the same. In the ABA example, the two “A” blocks could have different amino acid compositions, and even different glycine patterns. A more specific example of the miniblock polymers is shown below:

| B A C A D A D A B | |
|---|---|
| (EDE)₅(GAGAGS)₄RGDS(GAGAGS)₄PGP(GAGAGY)₂PGP(GAGAGS)₂(EDE)₄ | (SEQ ID NO:32) |

In this example, there are acidic solubilizing “B” blocks on either end of the oligomer. The middle portion “ACADADA” includes several turns, which will cause the molecule to adopt an “inchworm” structure. The RGDS “C” block is a specific integrin binding sequence found in extracellular matrices. Placed between two equally sized “A” blocks, it will probably act as either an exposed loop or as a turn, situations in which this particular binding sequence is most active.

Another example is the following amino acid sequence:

| “B” “C” “B” “A” “D” “A” | |
|---|---|
| NYNS (GCCCG)₃NSYS (GPGGTGPGQQGPGGTW)₂GPG (GVGVGSGAGAGD)₃ | (SEQ ID NO:33) |
| “D” “C” “D” “A” “D” “C” | |
| GEGP MKHMA EGPG (GAGAGYGAGY) GPG GESYRGDGSG | |
| “D” “A” “B” | |
| GPG (GVPGVAGGP)₃SYSSNR | |

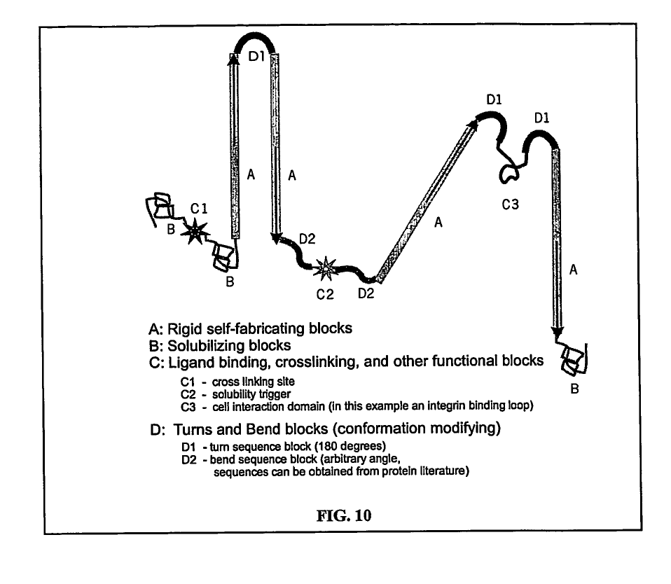

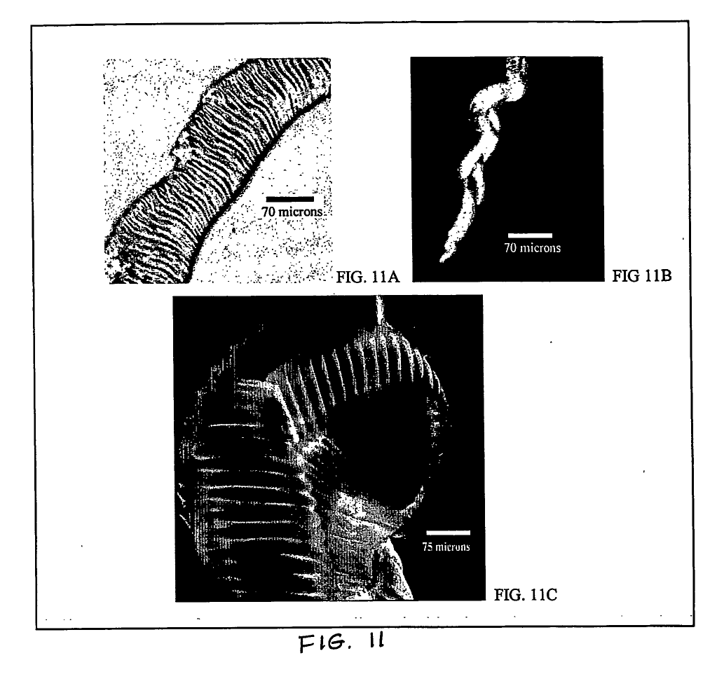

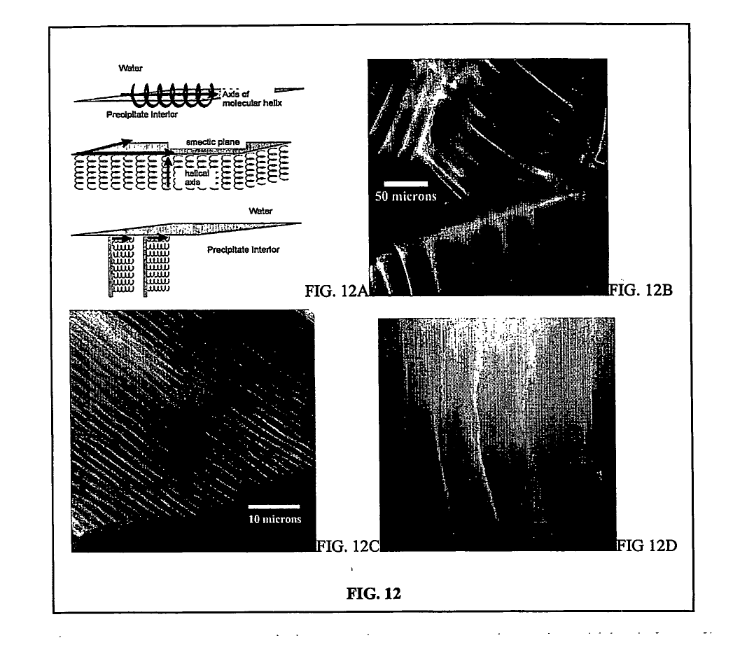

This sequence has a block structure (or block architecture) BCBADADCDADCDAB, as shown in boldface. A schematic of this oligomer example is shown in FIG. 10.

The miniblock polymers can be modified biopolymers such as modified native proteins or polysaccharides, or synthetic polymers such as recombinant proteins, polypeptides, polyesters, polyamides, polyimides, polyimines, or copolymers thereof. Examples of the polymers include collagen-like proteins and nylons.

The following sequences are examples of the miniblock polymers (more specifically, polypeptides):

| | |
|---|---|
| Based on Type 1 “A” blocks-aggregation transition | |
| (E)₅G (C)₂(GAPGPP)₅(C)₂G(E)₅ | (SEQ ID NO:34) |
| (E)₅GCCE (GAPGPP)₂GAPGPR-GDPGPP-GAPGPP(C)₂ G (E)₅ | (SEQ ID NO:35) |
| (E)₂CERGDE (GAPGPP)₅ERGDEC (E)₂ | (SEQ ID NO:36) |
| (E)₅(GAPGPP)₂ GCPGPP (GAPGPP)₂(E)₅ | (SEQ ID NO:37) |
| (E)₂G (C)₄(GAPGPP)₅(C)₄G (E)₂ | (SEQ ID NO:38) |
| (E)₂G (C)₄(GVPGPP)₅(C)₄G (E)₂ | (SEQ ID NO:39) |
| (E)₃(GCPGPC)₆(E)₃ | (SEQ ID NO:40) |
| (E)₃(GPAGPP)₄(E)₃ | (SEQ ID NO:41) |
| (E)₃(GAOGPO)₄(E)₃ | (SEQ ID NO:42) |
| (E)₃(GVOGPO)₄(E)₃ | (SEQ ID NO:43) |
| (E)₅(GPPGVP-GPPGPS-GPPGVP-GSPGPP-GPVGPS-GPP)(E)₅ | (SEQ ID NO:44) |
| (E)₅(GAPGPO)₆(E)₅ | (SEQ ID NO:45) |
| (E)₅(GVOGPO)₆(E)₅ | (SEQ ID NO:46) |
| (K)₅(GAPGPPGDP)₄(K)₅ | (SEQ ID NO:47) |
| N₅ (GPAGPP)₆N₅ | (SEQ ID NO:48) |
| N₅ (GAPGPP)₆N₅ | (SEQ ID NO:49) |
| N₅(GPVGPP)₆N₅ | (SEQ ID NO:50) |
| N₅(GVPGPP)₆N₅ | (SEQ ID NO:51) |
| NGSNN(GAPGPP)₆NGSNN | (SEQ ID NO:52) |
| N₅(GPAGPP)₃GPRGDP(GAPGPP)₃N₅ | (SEQ ID NO:53) |
| (E)₅(GVPGPV)₆(E)₅ | (SEQ ID NO:54) |
| (E)₅(GVPGPA)₆(E)₅ | (SEQ ID NO:55) |
| (GAPGPP)ₙ | (SEQ ID NO:56) |
| (E)₅(GVPGPP)₆(E)₅ | (SEQ ID NO:57) |
| (E)₅(GLPGPP)₆(E)₅ | (SEQ ID NO:58) |
| (E)₅(GIPGPP)₆(E)₅ | (SEQ ID NO:59) |
| (K)₅(GPPGPPGDP)₄(K)₅ | (SEQ ID NO:60) |
| Based on Type 2 A Blocks-coil to helix transition | |
| SAASAASAASAASAASAA | (SEQ ID NO:61) |
| SALSVASIASALSVASIA | (SEQ ID NO:62) |
| YALSVATIAYALSVATIA | (SEQ ID NO:63) |
| AAAAYAAAAYAAAAYAAAAY | (SEQ ID NO:64) |
| AAAAYAAYAAYAAYAAY | (SEQ ID NO:65) |
| AAASSIIIAAASIIIAAASS | (SEQ ID NO:66) |
| (E)₃(A)₁₅(E)₃ | (SEQ ID NO:67) |
| (E)₃(A)₂₀(E)₃ | (SEQ ID NO:68) |
| (E)₃(A)₂₂(E)₃ | (SEQ ID NO:69) |
| (EAAAK)₄ | (SEQ ID NO:70) |
| (EAAAK)₅ | (SEQ ID NO:71) |
| Based on Type 3 “A” blocks-folding into folded β-structures | |
| GIGIGS | (SEQ ID NO:72) |
| (E)₅(GAGAGD)₄(E)₅ | (SEQ ID NO:73) |
| (E)₅(GVGVGD)₄(E)₅ | (SEQ ID NO:74) |
| (E)₅(GLGLGD)₄(E)₅ | (SEQ ID NO:75) |
| (E)₅(GIGIGD)₄(E)₅ | (SEQ ID NO:76) |
| (E)₅(GVGVGY)₄(E)₅ | (SEQ ID NO:77) |
| (E)₅(GKGAGD)₄(E)₅ | (SEQ ID NO:78) |
| (E)₅(GAGAGT)₄(E)₅ | (SEQ ID NO:79) |
| (E)₅(GGAGGA)₄(E)₅ | (SEQ ID NO:80) |
| (E)₅(GGAGGT)₄(E)₅ | (SEQ ID NO:81) |
| (E)₅(GGAGGD)₄(E)₅ | (SEQ ID NO:82) |
| SGAGVGRGDGSGVGLGSGNG | (SEQ ID NO:83) |
| GDVGGAGATGGS | (SEQ ID NO:84) |
| GNVGGAGASGGS | (SEQ ID NO:85) |
| GGAGGGGTGGLGSGG. | (SEQ ID NO:86) |
| GGAGGGGTGGLGSGGAGAGGLGGGGAG | (SEQ ID NO:87) |
| GPGGTGPGQQGPGGTW | (SEQ ID NO:88) |
| GPGNNGPGGYGPGNNGPSGPGSA | (SEQ ID NO:89) |
| DPGVYGPSGNAPGVYGPSGQGAGAGS | (SEQ ID NO:90) |
| (E)₅(GAGAGS)₄(E)₅ | (SEQ ID NO:91) |
| (E)₅(GVGVGS)₄(E)₅ | (SEQ ID NO:92) |
| (E)₅(GIGIGS)₄(E)₅ | (SEQ ID NO:93) |
| (E)₅(GVGVGY)₄(E)₅ | (SEQ ID NO:94) |
| (E)₅(GAGAGY)₄(E)₅ | (SEQ ID NO:95) |
| (E)₅(GAGAGD)₄(E)₅ | (SEQ ID NO:96) |
| (E)₄(GAGAGS)₂(E)₄(GAGAGS)(E)₄ | (SEQ ID NO:97) |
| (E)₄(GAGAGS)(E)₄(GAGAGS)(E)₄ | (SEQ ID NO:98) |
| (E)₆(GAGAGD)₃(E)₆ | (SEQ ID NO:99) |
| (E)₆(GVGVGS)₃(E)₆ | (SEQ ID NO:100) |
| (E)₆(GIGIGS)₃(E)₆ | (SEQ ID NO:101) |
| (E)₆(GVGVGY)₃(E)₆ | (SEQ ID NO:102) |
| (E)₆(GAGAGS)₃(E)₆ | (SEQ ID NO:103) |
| (E)₅(GPGGYGPGQQGPGGY)(E)₅ | (SEQ ID NO:104) |
| (E)₅(GPGQQGPGGYGPGQQGPSGPGSA)(E)₅ | (SEQ ID NO:105) |
| (E)₅(DPGVY-GPSGQ-DPGVY-GPSGQ)(E)₅ | (SEQ ID NO:106) |
| GAIGGVGATGGS | (SEQ ID NO:107) |
| (R)₃(GGAGQGGYGGLGSQGAGRGGLGGQGAG) (R)₃ | (SEQ ID NO:108) |
| GVGVGS | (SEQ ID NO:109) |
| (E)₅(SGAGVG-RGDGSGV-GLGSGNG)₂(E)₅ | (SEQ ID NO:110) |
| (E)₅(GDIGGV-GATGGS)₂(E)₅ | (SEQ ID NO:111) |
| (E)₅(GNVGGA GASGGS)₂(E)₅ | (SEQ ID NO:112) |
| (E)₅(GVDGGA-GATGGS)₂(E)₅ | (SEQ ID NO:113) |
| GVGGGY | (SEQ ID NO:114) |
| GAGAGY | (SEQ ID NO:115) |
| GAGAGD | (SEQ ID NO:116) |
| SGRGGLGGQGAGGGAGQGGYGGLGSQG | (SEQ ID NO:117) |
| GAGAGT | (SEQ ID NO:118) |
| (SGAGVGRGDGSGVGLGSGNG) | (SEQ ID NO:119) |
| (R)₃ (GGAGQGGYGGLGSQGAGRGGLGGQGAG) (R)₃ | (SEQ ID NO:120) |
| (E)₅(GDVGGAGATGGS)₂(E)₅ | (SEQ ID NO:121) |
| (E)₅(GDVGGAGASGGS)₂(E)₅ | (SEQ ID NO:122) |
| (E)₅(GDIGGVGATGGS)₂(E)₅ | (SEQ ID NO:123) |
| (E)₅(GPGGYGPGQQGPGGY)(E)₅ | (SEQ ID NO:124) |
| (E)₅(GPGQQ-GPGGY-GPGQQ-GPSGPG-SA)(E)₅ | (SEQ ID NO:125) |
| (E)₅(DPGVY-GPSGQ-DPGVY-GPSGQ)(E)₅ | (SEQ ID NO:126) |
| Based on Type 4 “A” blocks-paracrystal formation | |
| SGRGGLGGQGAGMAAAAAMGGAGQGGYGGLGSQG | (SEQ ID NO:127) |
| MKHMAGAAGAVVGGLGGYMLGSAM | (SEQ ID NO:128) |
| M DAEFRHDSG YEVHHQKLVF FAEDVGSNKG AIIGLMVGGV | (SEQ ID NO:129) |
| VIATVIVITL VM | |
| (E)₄(VSSTGSTSNTDSSSKSAGSRTSGGTSTYGYSSSHRGGS)(E)₄ | (SEQ ID NO:130) |

Non-peptidyl miniblock polymers, e.g., polyamides, poly(ester-co-amide)s, poly(alkyleneoxide-co-amide), can be synthesized by known polymerization methods, such as controlled condensation polymerization ring-opening polymerization, or ionic polymerization. See, e.g., Odian, Principles of Polymerization, 3^{rd} Ed, John Wiley & Sons, Inc., New York, 1991; and Hatada et al. (ed.), Macromolecular Design of Polymeric Materials, Marcel Dekker, Inc., New York, 1997. Furthermore, normative amino acids can be incorporated into the miniblocks as desired.

Self-Fabrication of Miniblock Polymers

The miniblock polymers can be dissolved to prepare solutions in various types of media. The solubility of the miniblock polymers varies greatly with the nature of the miniblock polymers and solvents. For instance, hydrophobic polymers containing charge-free blocks are generally not soluble to high concentrations in aqueous solvents such as pure water, aqueous solutions (e.g., a Tris buffer), and mixtures of water and miscible organic solvents (e.g., methanol), and in organic solvents such as methanol or ethanol. Hydrophilic polymers with charged solubilizing blocks will be more soluble in polar solvents, but less soluble in non-polar solvents. The solubility of hydrophobic polymers in alcohols with a higher number of carbon atoms (e.g., propanol and hexanol) decreases with the increase of the carbon atom numbers of the alcohols. Dissolution of the miniblock polymers allows them to swell for further functionalization (e.g., chemical modification of the side chains) and processing (e.g., casting films).

After dissolving in a medium and under certain conditions (e.g., at 0° C., 1° C., 2° C., or 3° C.), the “A” blocks cause the miniblock polymers to change their molecular conformations, e.g., from good extension to compact structures such as hairpin and helix. The compact character of the polymers subsequently facilitates alignment and aggregation of the polymer chains, and initiates self-fabrication of the miniblock polymers to form self-fabricated materials. The self-fabricated materials have a long-range order, and sometimes precipitate from the solutions or can be obtained as a glassy single phase nanopatterned material (depending on whether a strong precipitant is used to induce self-fabrication).

A key feature of the self-fabrication processes is the presence of different types of “transformations” (e.g., folding and aggregation) which can be forced to occur simultaneously. Competition and interaction between the transformations results in a large variety of selectable nanopatterns, which can be incorporated into the self-fabricated materials. For example, the transformation from a liquid, which is merely oriented, to a liquid comprised of highly oriented layers of molecules, changes the liquid from one type of long, range order to another. Conformation and aggregation transitions change the shapes of miniblock polymers and the side chains of the miniblock polymers, which in turn alters the solubility of the miniblock polymers.

Simultaneous transformations of miniblock polymers can be forced to happen by, e.g., concentrating their solution to the concentration at which orientation and layered structure normally occurs for the aggregate structure while simultaneously cooling the solution to induce triple helix formation. For instance, a saturated or supersaturated solution of a miniblock polymer, e.g., a polypeptide, can be first prepared by placing the polypeptide in a mixture of ethanol (or methanol or propanol) and water where the solvent system has compositions ranging from 0% water and 100% alcohol to 0% water and 100% alcohol. Alternatively, the polypeptide can be placed in an aqueous ammonia solution with a pH value of 7-8.5, or in an acetic acid/water or formic acid/water solution such that a swollen polypeptide precipitate and dissolved polypeptide coexist in the solvent. The mixture is gently shaken and allowed to age in sealed plastic (hydrophobic) containers for 0-5 days during which time a solid precipitates and a thin film forms at the air-solution interface. The thin film can be collected from the air-solvent interface and precipitated by using alcohol, glycol, or acetone, followed by treatment with an acid and then an organometallic compound. The solid precipitate can be recovered by filtration, depositing onto glass, mechanical removal of precipitate, adsorption onto a glass rod or plate swirled through the mixture. An organic solvent can be used to release the solid precipitate from the adsorption surface.

The self-fabrication process can take place in a variety of solvents under a variety of conditions and sample preparation geometries to form films, fibers, thick materials, ribbons, or microscopic coiled springs. The self-fabricated materials include subtextures that persist down to a few nanometers. Examples of the subtextures include regular stripes or bands comprised of smaller bands (which can be parallel or at varying angles to the larger structures) where the smaller bands are comprised of yet smaller periodic structures in a hierarchy of decreasing length scales producing ordered arrangements down to the molecular and submolecular levels. This type of hierarchical order is possible in smectic, hexatic, and other types of liquid crystalline phases (or plastic crystalline or ordered fluid) where chiral molecules form the major component of the ordered phase.

The formation of thin textured films is due to the blocky structure of the miniblock polymers in which solubilizing blocks (i.e., “B” blocks) alternate with self-fabricating blocks (i.e., “A” blocks) or other crystallizable or rigid structure forming blocks. For instance, a triblock polymer having two hydrophilic charged solubilizing end blocks (i.e., “B” blocks) and a hydrophobic self-fabricating core block (i.e., “A” block) could remain soluble and repulsive under most conditions. Such a design could also fold into rigid H-bonded “hairpins” when the charge-charge repulsion in the end blocks is sufficiently reduced through a change in temperature, concentration, or chemical environment of the triblock polymer. For polymers which form rigid structures through folding into hairpins, there are charged solubilizing ends which can be repulsive under one set of conditions and which collapse (recombine with counter ion) to form polar attractive structures under a second set of conditions. In this case folding into a hairpin is driven by polyelectrolyte collapse, which will be very strongly concentration, pH, and solvent polarity driven. The point at which each individual polymer will experience solubilizing end group collapse and start to fold can be predicted through calculations of pKa (for an acid example) and through pH titration measurements. Temperature and “A” block sequence length will also affect the folding rate and will have a minor effect on the concentration and pH at which hairpin formation occurs. For example, if the B block is (DEDEDED) (SEQ ID NO: 131) and the A block is (GAGAGS GAGAGS GAGAGS) (SEQ ID NO:132), the hairpins will form at a concentration of approximately 100 mg/mL and a pH of approximately less than 3. The rigidity of the hairpin structure provides an asymmetric unit for oriented packing of molecules and liquid crystal formation. The chemical disparity between the different domains in such a mini triblock polymer stabilizes smectic organizations over other liquid crystalline types of organization, since the end blocks occupy a different material domain than the chemically incompatible middle blocks in a smectic organization. The chirality of the molecules (little folded helices with a preferred handedness) results in a tendency for the smectic layers to twist and produces a visible texture in the films.

The self-fabrication process is strongly temperature-dependent. The fabrication temperature of a polymer, i.e., the temperature at which a polymer self-fabricates, can be affected by the chemical composition and sequence (or building blocks) of the polymer. For instance, polypeptides with glutamic acid end blocks will fabricate in the 0-3° C. range. Polymers with different end blocks, as well as different core helical blocks (e.g., prolines vs. hydroxyprolines) fabricate at different temperatures. For examples, polypeptides with asparagine end blocks have a higher temperatures (associated multistrand rope in the “A” block) and milder pH or lower concentration (hairpin structure “A” block) fabrication window than polypeptides with glutamic acid end blocks; and polypeptides with a hydroxyproline-containing core fabricating block have a lower fabrication window than polypeptides with a proline-containing core fabricating block. Self-fabricating oligopeptides with an “A” block which forms multistrand associated ropes have a higher fabrication window when the core fabricating (“A”) block contains hydroxyproline than when only it contains non-hydroxylated imino acids (pralines). The stability (in particular, the thermodynamic stability) of a particular conformation (e.g., helix) is a function of the ensemble entropy (e.g., water and solutes). Thus, in the case of polymers which self-fabricate through association into multistrand helical ropes, helix stability will depend on molecular interaction within the triple helix regions. It will also include a large contribution from interaction with water and from changes in solvent entropy. Solvent entropy is expected to be strongly dependent on the geometry of interaction with the polymer. This geometry is a convolution of sequence and sequence pattern and helical conformation and spatial pattern. Thus, the range of stability and triple helix conformations favored could be quite different for sequences with small differences. For example, a miniblock polymer containing the sequence of GAPGPP (SEQ ID NO:133) would have a lower thermal stability than a miniblock polymer containing the sequence GPAGPP (SEQ ID NO:134).

Repetitive blocks in the miniblock polymers are likely to have higher and wider fabrication temperature ranges than nonrepetitive polymers, unless specific geometric interactions are designed into the non-repetitive sequence (for example an acid base bridge in the hairpin or triple helical state). In addition to folding blocks, the polymers can also contain non-folding blocks which localize guest molecules (e.g., dyes or electromagnetic signature modifiers) either at the end or in the middle of the polymer chains. It is worth noting that properties of these non-folding blocks (e.g., their sizes, helix forming tendencies, and chemistry) can also be used to tune the structures and properties of the polymers. More specifically, incorporating non-folding blocks (e.g., oligoglutamic, oligomethiones, Pro-Ala-Pro) in the center of a polymer chain lowers the effective length and rigidity of the rod (mesogen). For a short non-folding block, this feature should result in a lower chirality due to loss of rigidity. For a long non-folding block, higher chirality can be obtained if the two-rod segments that are separated by the spacer behave semi-independently. Changes in overall chirality are used to manipulate the periodicities of orientation and chemistry in the self-fabricated materials at the nanoscale and at higher length scales. A lower chirality will typically result in a longer material periodicity. The periodicities in the molecular chemistry and orientation affect the manner in which the material responds to infrared radiation when the material is prepared from a chiral smectic (nanolayered) phase In many nanolayered materials, where the nanolayers twist to form a regular pattern, infrared radiation does not travel in a straight line from source to sample to detector, as observed in spectrometry experiments. The wavelengths in the infrared region affected are different from those affected through normal infrared absorption by a non-self-fabricated polymer material with similar gross dimensions and composition. Instead, a broad range of the infrared spectrum, typically in the 1-15 micron region, is strongly attenuated. The shortest wavelengths affected are correlated to the periodicities in the self-fabricated material, where a shorter material periodicity results in infrared spectrum modification down to a shorter wavelength. The material periodicities and shortest affected wavelengths can be nearly (but not exactly) identical for materials based on collagen-like triple helices. The correlation between infrared response and material periodicity for other self-fabricated materials, such as the hairpin structure based tapes in FIG. 9, is more complex. It appears that in these materials the range of modified wavelengths is bracketed by the smallest and largest strong periodicity in the material. Well-defined geometric interactions between end groups in adjacent layers result in correlation of 3-dimensional molecular orientations and may influence the self-fabrication of the polymer.

Other modifications that can increase the temperature at which self-fabrication occurs include: (1) incorporating longer folding blocks to enhance thermal stability, (2) cross-linking the polymers by, e.g., metal bridges, disulfide bonds, or covalent coupling (by enzymatic methods or chemical cross-linking, e.g., using glutaraldehyde and carbodiimide); and (3) chemical modification of the side chains in the self-fabricating blocks—for example fluorination or (in the case of imino acid rich triple helix forming blocks) increasing the hydroxylation level and therefore the thermal stability of certain residues or building blocks (e.g., prolines) in the polymers.

The chirality, which depends on conformations and sequences, of the fabricating blocks controls the twisting of the self-fabricated materials. The layered structure, twisting, and chemistry of the material, including any guest molecules to functionalize sublayers, determine the physical chemical, mechanical, thermal, and optical properties of the materials. The conformation (e.g., helix) and degree of the amphiphilicity of the polymer sequence control the nanoscale patterns present in the materials, the nanoscale chemistry of the materials surfaces and the tendency to form ordered materials.

The sequence or building blocks of a polymer also affect the fabrication temperature of the polymer. For instance, a polymer having the sequence GPAGPP (SEQ ID NO:135) forms a self-fabricated material at 4.5° C., while a material fabricated from a polymer having the sequence GAPGPP (SEQ ID NO:136) has a highest self-fabrication temperature of 3° C. There are a number of temperature ranges for each polypeptide, each giving different types of self-fabrication behavior (e.g., a cholesteric material at 4.5° C. and a smectic material at 2-3° C. for GAPGPP (SEQ ID NO:136).

At temperatures below 10° C. hairpin formation will be inhibited in miniblock polymers with very short “A” blocks. The low temperature crystalline form for the “A” block is an extended structure, and crystallization of the extended form can compete with hairpin liquid crystal formation. Solvents of low polarity will not fully ionize charged ends, resulting in more polar attractive behavior. Some low polarity solvents will also affect the conformation of the “A” blocks. Thus, there are a range of distinct parameters which can be tuned to force particular structures from the molecules and to use these structures to create nanopatterned materials. Many of these parameters (e.g., temperature concentration and solvent polarity) can be varied almost independently, allowing multiple aspects of nanopatterned material formation to be controlled at once. For example, for an acid terminated hairpin, a high concentration of the polymer in a polar solvent can be used to ensure layer formation in the flexible surfactant state. The temperature can be selected, e.g., to favor a tilted smectic nanolayered structure when the molecules are folded into hairpins. An alcohol can be added to the solvent to lower the solvent polarity and force hairpin formation under specified temperature and concentration conditions. Different grain sizes and materials structures can be tuned by inducing hairpin formation in different parts of the temperature or concentration range. Chemical and morphological complexity allowing tunable materials structures are another key feature of the invention.

It is also possible to have uncharged polar solubilizing blocks in the polymer. The chemical complexity and material complexity is lowered in these types of polymers, resulting in simplified self-fabrication behavior. The self-fabrication of these molecules into materials is still tunable, but parameters such as temperature, concentration, solvent polarity, and pH are no longer well separated in their influence on different stages in self-fabrication. Thus, these polymers have less tunable self-fabrication pathways, but form particular well-defined material structures (defined in terms of molecular orientation, layer spacing and geometry, grain size and material domain structure) more robustly in wider ranges for each parameter. The self-fabrication process is quicker for these polymers due to the reduced chemical complexity.

It is also possible to “invert” the chemistry of the self-fabricating “A” blocks and solubilizing “B” blocks so that the “A” block is hydrophilic or charged and the “B” blocks are hydrophobic. Self-fabricated structures will still form in polar and non-polar solvents, but the small scale material patterning and the chemical resistance of the materials will be different in the chemically “inverted” polymers. For instance, a slightly hydrophilic “A” block could be combined with two hydrophobic “B” blocks to self-fabricate materials which resist polar solvents, and to incorporate hydrophobic “guest” molecules into the nanolayered structure. In a polar solvent, stable micelles and vesicles will form, whereas in a nonpolar solvent, smectic layers are expected.

Changes in molecular weight of the new miniblock polymers can also affect the fabrication temperatures and concentrations of the polymers. An increase in the molecular weight will increase the fabrication temperature for polymers that can associate into multistrand ropes. For polymers that can form hairpins, an increase in block length will lower the concentration of the miniblock polymers required to induce self-fabrication. In this case, an increase in molecular weight will slow bulk self-fabrication, but will speed processes based on interfacial oriented adsorption of the polymer. In particular, increases in the solubilizing block size or molecular weight facilitate the self-fabrication process by increasing the concentration of the polymer in solution. If the solubilizing blocks are not charged (and thus not repulsive), but are instead polar and attractive, longer blocks will attract each other more and favor self-fabrication, increasing the fabricating temperature. Solvent conditions can be used to tune and control the association (or aggregation) of miniblock polymer molecules that are well separated in the linear sequence. For instance, when a hairpin-forming triblock polypeptide with charged end blocks is dissolved at a supersaturated concentration in ethanol or methanol, the polypeptide would undergo a reduction in charge on the charged ends, which favors the formation of hairpin structures. Ethanol and methanol, however, would be poor solvents overall for a triblock polypeptide having charged “B” blocks as end blocks and a glycine-rich hydrophobic “A” block as the center block, as these alcohols are poor solvents for the core miniblocks and good solvents for the acidic end blocks. In a disordered precipitate (i.e., in a polypeptide that has been purified and lyophilized, but not further processed), there would be a network of hydrophobic regions resistant to dissolution, as well as a network of acidic regions which do dissolve, resulting in a swollen material in which each molecule undergoes a cooperative rearrangement to dissolve. The cooperative rearrangement of the polymer molecules results in the formation of a smectic organization within thick swollen precipitates, where the smectic will slowly readjust itself to a more perfect structure over time if the solution conditions remain the same. The slow time scale of rearrangement after the initial formation of textured films provides an ample time window to control the morphologies obtained and produce, for example, nanolayered structures with different patterns and length scales of twisted boundaries between layered domains (as in the textured tapes in FIG. 9). Alternatively, one could favor ordered arrays of spherulitic structures over a completely homogeneous thin multi-layered film.

Different blocks can be selected to form the new miniblock polymers and to define interlayer regions with small but finite nano-scaled dimensions that are chemically and physically (i.e., geographically) distinct from the regions defined by chemically disparate blocks or sequences. Thus, each smectic layer of a rigid folded miniblock molecular structure also contains chemically distinct sublayers consisting of different miniblocks. As the orientation of the smectic layers change with respect to the surface of the material, the sublayers are alternately buried inside the material and exposed in nano-scaled alternating patterns of chemically distinct stripes at the surface. The result is a set of chemically patterned regions (within the stripes) having a small nano-scaled (1-5 nm) order and spacing controllable by the lengths of the blocks in the miniblocks. The relative lengths of the miniblocks and the total length of the fabricating blocks (i.e., “A” blocks) also control the spacing of the larger textures within the hierarchy of length scales and the degree of periodic misorientation of smectic layers leading to larger scale textures. Aging of the smectic layers in solvent or buffer, temperature, and concentration can also be used to control the spacing of the large-scaled geometric pattern, as it approaches the “perfect” geometry determined by the chiral parameters of the miniblock polymers.

In miniblock polymers, the heterogeneous nature of certain or all blocks allows for variation in the sequences and functions of the miniblock polymers, as well as incorporation of useful moieties, such as ligand binding epitopes, to the miniblock polymers. In addition, the low to moderate total molecular weight and small to moderate size of the miniblock polymers favor smectic like and liquid crystalline driven types of organization (through less prohibitive entropy reduction and then aggregation), rather than microphase separated (unoriented) nano-domains occurring in high molecular weight synthetic block copolymers. The small to moderate size of the rigid units in the folded polymer molecules favor self-fabrication of the polymers in a reasonable time scale, with a relatively low occurrence of errors, mismatched orientations, and domain walls and boundaries as compared to a polymeric liquid crystal. The heterogeneous chemical nature of the miniblocks and consequent high solubility of selected segments result in enhanced transport in a concentrated solution, in which polymer molecules will be expected to diffuse preferentially in a linear fashion and the soluble regions can “lead” diffusion modes such as reptation and aid in cooperative desorption from the bulk.

The self-fabricated materials typically are of a three-dimensional structure and are robust. See, e.g., FIGS. 4A and 4B, which show field emission scanning electron microscope (FESEM) images of a repetitive peptide having a smectic layered structure. The miniblock polymers that comprise these materials can be in various conformations. For instance, the miniblock polymers can be rigid rods and in amphiphilic liquid crystalline phases, in which the polymer molecules are oriented in a long-range order. The miniblock polymer molecules also can be of a hairpin structure and aggregate to form micelles or vesicles.

The self-fabricated materials have large domain sizes and novel domain patterns and oriented textures prepared from polymers. They can easily change from flexible amphiphilic behaviors to rigid rod-like liquid crystalline behaviors through self-fabrication of the folded polymers into various conformations such as associated multimeric rods. For instance, folding blocks that are flexible can associate into a rigid double or triple helix. The large size of the polymers comprising a self-fabricated material results in mechanical stability of the liquid crystal and facile preservation of orientation, morphology, and chemical orientation in fabrication and processing of material.

Although the self-fabrication process of the new polymers generally can start automatically, it can also be initiated by changes of ambient conditions (e.g., the solvent polarity, the pH and ionic strength of the solution, concentration, temperature, and dielectric and magnetic fields). Other factors that affect the self-fabrication of the polymers include the sequences of the polymers, side chains, hydrophobicity or hydrophilicity of the end and internal blocks, molecular weights, solvent properties, and trigger activation. Additional factors include disparities in the volumes of monomers or monomer side chains, chemistry or hydrophobicity in water of monomers or monomer side chains, ability of monomers to form specific interactions such as hydrogen bonding (especially with solvent) or acid-base interactions, interactions between monomers that stabilize the helix such as hydrogen bonds, acid-base or charge-charge interactions, and charge repulsions. It is also useful to have a core helical block in which several molecules participate to form a rigid rod, or a multimeric coil.

Note that changes in layered architectures and optical response of the self-fabricated materials can be externally driven post-fabrication. Examples of driving forces include temperature, pH, and electric fields.

The self-fabricated materials in solution usually exist in a liquid crystalline phase. After drying, the materials are stable and robust. Because of their specific helical structures, the polymers, as well as the materials made from them, can be used in molecular recognition. For example, the polymers can be used as biomaterials with medicinal activity incorporated as an inherent feature of the self-fabricated materials.

While the polymers can easily self-fabricate, the self-fabricated materials can also easily de-fabricate in response to a change in the ambient conditions such as temperature. This de-fabrication process, however, can be controlled by stabilizing (thereby reducing the tendency to de-fabricate), or prevented by locking (thereby permanently preserving), the conformation of the self-fabricated materials.

Stabilizing the self-fabricated materials, thereby reducing their tendency of de-fabrication, can be achieved by cross-linking the self-fabricated materials with, e.g., sulfur or disulfide compounds, or chelating agents such as Co²⁺ or Ni²⁺, that form a bridge between charged groups in the self-fabricated materials. In the case of collagen-based polymers, the stabilization can also be achieved by increasing the hydroxylation level of the polymer, e.g., by replacing proline with hydroxyproline. The stabilization of a liquid long range order to yield a solid material can be further achieved by mechanically “freezing in” organizations within the material via drying the solvent faster than the relaxation time of the polymeric molecules (slow) when the helical segments are associated into multimeric helices and are rigid, and via self-fabrication of helical segments into triple helices or formation of paracrystalline networks of H-bonds or other specific interactions between flexible unassociated (non-multimeric) helices in the flexible amphiphilic state. These stabilization processes, if initiated before the polymers self-fabricate, will increase the fabrication temperatures of the polymers.

Locking the self-fabricated materials, thereby permanently preserving the conformation of the polymers, can be achieved by, e.g., cross-linking the self-fabricated materials with a monomer that copolymerizes with the polymers, a peroxide, or radiation such as UV radiation, ionizing radiation, or γ radiation.

Characterization of Miniblock Polymers and Materials Fabricated Therefrom

The miniblock polymers and materials fabricated from them can be characterized by methods commonly used in the art. For instance, the polymers can be analyzed using nuclear magnetic resonance (NMR) spectroscopy or X-ray photoelectron spectroscopy (XPS) to determine their helical structure and chemical composition. The chemical composition of the polymers can also be analyzed by mass spectroscopy (MS), optionally combined with liquid chromatography (LC) or gas chromatography (GC). The molecular weight of the polymers can be determined by gel permeation chromatography (GPC). The microstructure of the materials, in dried form, can be observed with a high resolution a field emission scanning electron microscope (FESEM).

In addition, arrays of the miniblock polymer sequences can be screened quickly by testing for birefringence in a semi-thick film. For controlled thickness materials, one can measure the total amount of FTIR wavelength radiation that passes through a sample compared to a reference protein in a wavelength range of interest.

For controlled thickness materials, one can measure the total amount of FTIR wavelength radiation that passes through a sample compared to a reference material in a wavelength range of interest. Formation of large particles (e.g., films, fibers, and tapes) can be determined in saturated solutions by using light scattering.

“Slow” detailed screens include microscopic examination of solutions and suspensions (wet and dried), X-ray scattering (SAXS, WAXS) of materials to ascertain layer structure and layer and molecular orientation, TEM to determine layer structure and orientation, SEM to obtain surface pattern and layer structure, polarizing optical microscopy to determine birefringence, ellipsometry to obtain quantitative optical birefringence, detailed FTIR studies to determine specific IR response.

Applications of Self-Fabricated Materials

Because of their long-range order, the self-fabricated materials have shown enhanced optical properties relative to disordered protein, peptide, and polyamide glasses (in the case of oligopeptide polymers) and in general optical properties are enhanced over non self-fabricated polymeric materials with similar composition. Such properties include optical polarization, directional polarization and transmission of light, and redirection of light. These properties are especially notable in the mid-infrared region (2-10 microns), where many of the self-fabricated polymeric materials block the straight line transmission of an infrared signature. The most likely mechanism of the materials' reduction of the infrared signature, transmitted directly in a straight line, is through redirection of the radiation along a preferred materials direction, or through a change in the wavelength of the infrared signal. These materials therefore can be used to modify and improve the performance of IR-sensitive devices such as IR sensors, IR filters, night telescopes, and thermo-sensitive detectors. They can also be used as novel optical materials with high order nonlinear optical properties in the visible and infrared regions, dispersive polarizing elements, and as attenuating gratings in the optical and infrared wavelength ranges.

The miniblock polymers can be used to prepare chemically patterned templates to create patterned films of inorganic, organometallic, or optically active material. One can tailor the polymers to be used by choosing blocks which preferentially interact with and bind to the second component (thus forming a pattern that preferentially binds to the second component). Additional details can still be included in the templates by using a nanolithographic process, but with a great savings in time and cost. Second components such as optically active molecules could be bound into rigid oriented portions of the films, orienting them as well. The miniblock polymers can also be used to prepare chemically patterned templates with either general features (hydrophobiclhydrophilic) or specific features (cell binding epitopes such as RGD) to study the effect of patterning and length scale on cells. Topography and chemical pattern can be independently controlled.

Chemically patterned templates can be used in combination with the miniblock polymers and materials fabricated from the polymers to create patterned films of inorganic, organometallic, or optically active material by choosing blocks that preferentially interact with and bind to the second component, such as a metal, metal halide, organometallic compound, drug, light harvesting molecule, sensing molecule, molecule that binds specific analytes, magnetic rare earth ion, organic, organometallic, inorganic salt, or tissue component Chemically patterned templates with either general features (hydrophobic or hydrophilic) or specific features (cell binding epitopes such as RGD) can also be incorporated into the miniblock polymers before they self-fabricate to study the effect of patterning and length scale on cells. Both the topography and the chemical patterns of these self-fabricated materials can be independently controlled.

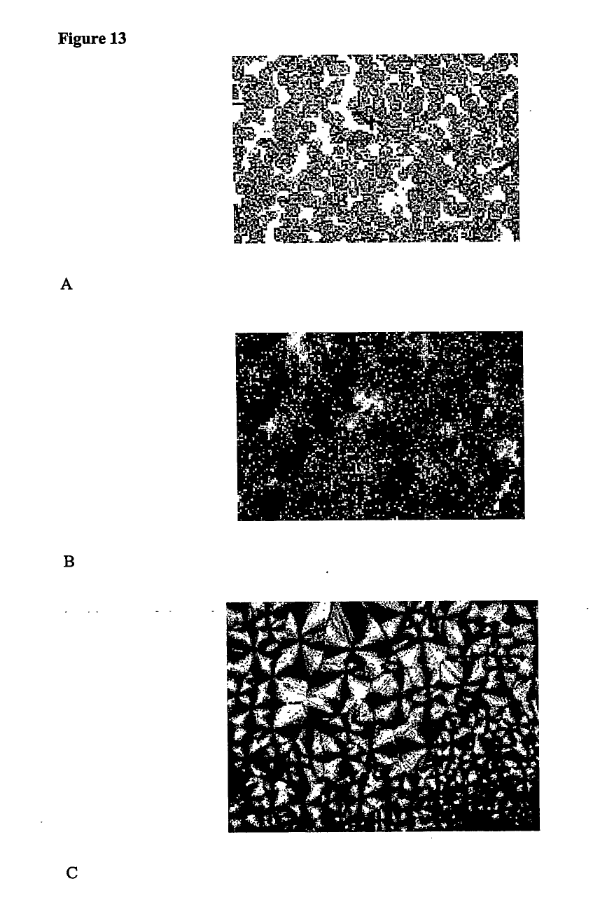

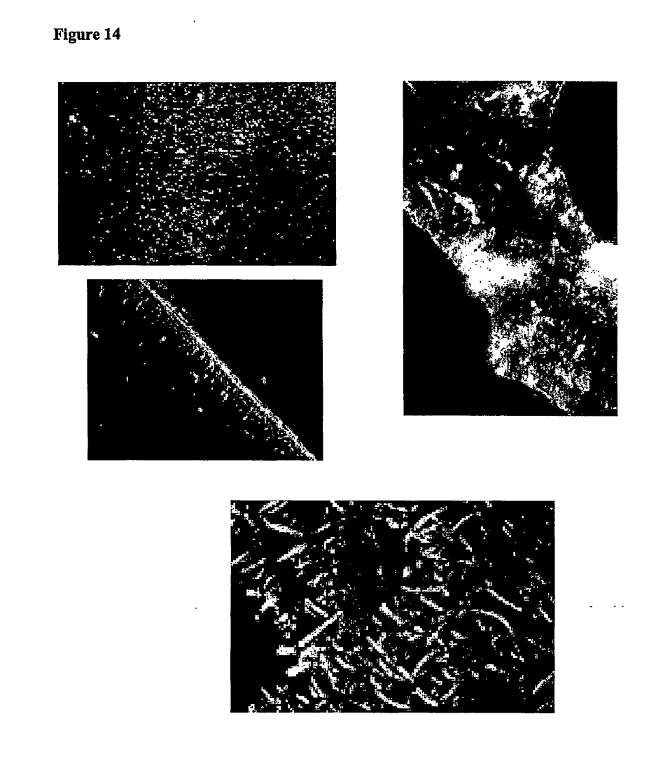

FIGS. 13 and 14 depict peptide liquid crystals with Rubipy. Crystals of Rubipy which grow after some of the compound has been taken up into the peptide liquid crystal are curved, highly birefringent, and chiral. In the polarizing optical microscope, an extinction line is observed which moves through the crystal when the sample is rotated. The handedness of the extinction line rotation always matches the handedness of the crystalline curvature. There are chiral crystals in the material expelled/excluded from the liquid crystalline phase. Racemic crystals grow in close proximity to the liquid crystal, i.e., where there is peptide present, but no ordered phase with which the Ru compound may interact. The complementary handedness to the Ru complex likely resides in the peptide liquid crystal. Furthermore, the presence of racemic crystals in peptide-rich isotropic sample regions indicates that simple interactions between peptide molecules and growing Ru compound crystals are not sufficient to give chiral crystals; in other words, this result is not an artifact of contamination of Ru compound crystals by peptide. Supporting this surmise is the observation that identical Ru compound crystals are observed in the excess Ru compound excluded from ordered phases of a variety of peptides, not all of which are collagen-like.

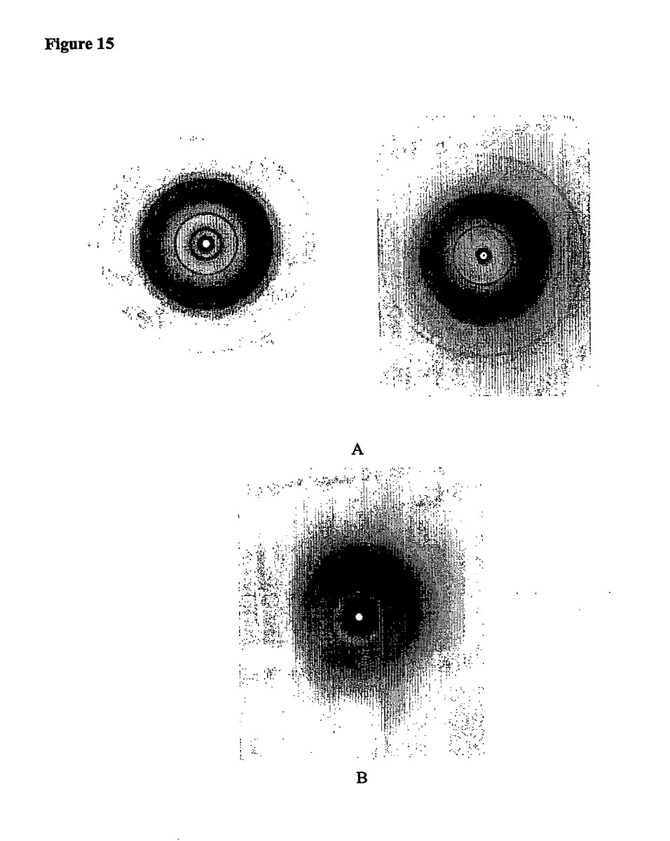

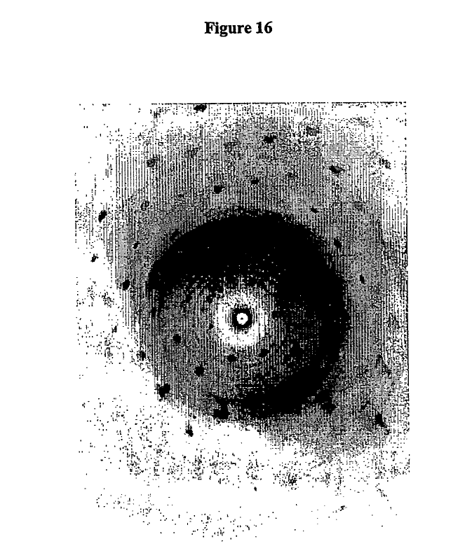

FIGS. 15 and 16 depict x-ray patterns for a pure Es[GSPGPP]6Es peptide flake and patterns of peptide+Rubipy crystals prepared under different conditions. Samples were prepared by drying slowly from aqueous solution in tubes with rounded bottoms. Note changes in relative intensity throughout the pattern of 15A (there is a background contribution from the Capton polyimide mounting film in the innermost ring of the figure at left). FIG. 15B depicts the x-ray pattern of a peptide+Rubipy crystal prepared by slow drying at 3° C. Again, there is a background ring on the innermost reflection from the Capton mounting tape, but arced Rubipy and peptide reflections (indicating orientation) can be seen at higher angles (larger radii). FIG. 16 is a sample of the same peptide+Rubipy solution, but dried at 1 C. In this case, faint features are visible near the center, and there is a lot of order, resembling a crystalline lattice of rel-rods (the smudgy spots in the lower right hand part of the pattern). There are also some typical peptide refelctions (rings), many of which appear to be arced indicating orientation. Rod-shaped reflections in an x-ray pattern indicate planar diffracting lattices—a layered arrangement. A set of reflections that is merging into a rod (suggesting smectic layering) is denoted with an arrow in FIG. 16.

The peptide reflections in the Ru-bipy samples are weaker than in the pure peptide, especially at high angle. It looks like there is noticeable absorption from the Ruthenium. Most of the pure peptides undergo a smectic A* to C* or a smectic C* to hexatic transition in the 1-3° C. temperature range (depending on the sequence).

The pure peptide in FIG. 15A at 3° C. should be in a smectic phase, but the acid-acid repulsive interactions between chain ends should reduce the interactions between layers and the overall order in the sample. In other words, peptide rods in one layer don't “know” the orientation of peptide rods in the next layer; at least in this case, there are no layer-to-layer orientation correlations. A tendency to twist may also result, leading to small ordered domains and very weakly oriented wide angle (i.e., small d-spacings) diffraction patterns.

When the Rubipy is added in FIG. 15B, acid-base complexation takes place. The peptides have acid block ends which when filly ionized have a charge of −30 per triple helix. Rubipy is a base with a +2 ionized state. We calculated the concentration of Rubipy in the sample to balance the charge of fully ionized peptide triple helices. Acid—base salting out between the peptide rod chain ends and the rubipy (approx. 1 nm diameter), is expected to reduce greatly the volume of the peptide end blocks and thus to also reduce any twisting which happens to accommodate the volume of the chain ends as compared to the dense (and thus small) triple helices. We would thus expect larger domains and more regular flat layers in a smectic phase. Our polarizing optical studies indicate that Rubipy/peptide complexes and composites follow the smectic phase behavior of the peptide, so this composite should be in a well-ordered smectic A phase and show evidence of uniaxial orientation and layers. We see evidence of this aggregation in the diffraction pattern. We also see sharp sampled rings with reasonable d-spacings for Rubipy and more diffuse rings with hk0 d-spacings typical of the pure peptide. This indicates that the two species are co-oriented and that the presence of Rubipy changes the layer thickness of the peptide complex (versus pure peptide), but not the interchain distance within the layers. We thus have strong evidence from both wide angle X-ray diffraction and from polarizing optical microscopy that the Rubipy segregates into the inter-layer end-block rich region. Therefore, this layered nanocomposite is a completely self-assembled peptide/organometallic.

In FIG. 16, we again see two diffraction patterns from a red (from Rubipy) peptide Rubipy composite with no discernable evidence for separate crystallites in the polarizing optical microscope. At this temperature, the peptide enters a hexatic phase (1° C.), which is expected to be more perfect or even paracrystalline with the addition of the organometallic base (Rubipy). We see hexagonal spots which smear out into sampled streaks at the edges of the diffraction pattern. If we have a layered sample, we have a long superlattice which results in closely spaced diffraction spots in directions parallel to the layer normals. Imagine a layer thickness of 10 nm. We would see multiple orders of this thickness where the second order has a d-spacing of 100/2=50, the third is 100/3=33, and so on to include the 6^{th} order (18A), 7^{th} order (15 A) 8^{th} order (12 A), etc. where the orders become more and more closely spaced as we get to the small d-spacings observed in a WAXS pattern. The overlap of these reflections causes them to smear out into rods as shown in the arrow in the figure. As would be expected from a more ordered layered nanocomposite, we observe a well ordered layered pseudohexagonal crystal of the peptide, induced by the presence of Rubipy, and misoriented Rubipy domains, i.e., the rings, which are small.

This technique for making peptide-inorganic, peptide-organometallic, and peptide-organic nanocomposites appears quite general. In fact, we have achieved similar results with a number of different peptide rigid rod cores and small molecule and ionic bases, including magnetic rare earth ions and organic compounds, etc. Thus, we have a peptide “toolkit” with a range of different chiralities, layer thicknesses, interlayer region widths, orientation patterns, chemical and physical interactions for segregation of the second phase, orientations, net dipoles, etc. This molecular “toolbox” provides an enormous flexibility in building highly repetitive patterned composite materials with predictable nanoscale features. The free surfaces of theses materials and their glassy fracture surfaces provide lines and grids which are regular and provide chemical patterning over areas similar to dip pen nanolithography and other brute force techniques. However, this chemical approach easily creates pattern with a basic element (or unit cell) less than 100 nm in diameter, and has been used to create patterned arrays of lines down to 1-5 nm. Since thermodynamics is the driving force for forming the patterns, they are stable to spreading, and many of the materials retain their layered structures to temperatures in excess of 150 to 200° C. Because wetting and spreading are non-issues, a large variety of chemistries can be incorporated into the patterned arrays easily, whereas brute force writing methods require that the entire process be optimized for each new molecule being used to write.

The materials can be used for preparing films, membranes, or coatings that absorb specific wavelengths of infrared radiation. These coatings or films can be made by methods that include preparing a chiral polymeric material containing multimeric associated helical segments, nonhelical segments, attachment sites for metal, inorganic, or other optically active moieties in either the helical or non helical domains of the polymer, and initiating the self-fabrication of the polymer to form an IR responsive chiral smectically ordered material. The material is swollen with a poor solvent (such as propanol) for incorporation of additional chemical moieties.

The materials can also be used in optical applications. For example, they can be useful as matrices to align non-linear optical (NLO) chromophores, which are useful in creating materials for second order nonlinear optics, such as frequency doubling materials to increase data throughput in optical cable. In addition, if a generalized scheme for organic chromophore alignment can be attained, a large number of efficient, cost effective optical switching, mixing, demixing, and filtering materials are possible. Such materials can form the bases for logical devices used in optical computing and would facilitate commercialization of optical computing technologies.

There are novel features of helical biopolymers that help them to bind to chromophores in well-defined locations and orientations relative to the helical biopolymer, to deliver chromophores at a high volume fraction, to achieve a polar orientation of molecules in a potable liquid state, and to create an ordered, oriented, thermally stable solid from the pooled liquid state. Specific materials that can be used include those selected from sequences based on three separate classes of proteins, all known to form highly aligned structures with a polar orientation and net electrical dipole moment in vivo. The three classes are designed oligopeptides based on collagen, keratin, and silk. These three types represent a range of molecular dipoles, a range of steric interactions or shape factors favoring parallel packing (working against the tendency for dipoles to randomize), a range of molecular stiffness, and a range of diameters and smallest stable helix lengths.

A high dipole will resist polar orientation in an undisturbed liquid, but will orient more strongly in an applied field, whereas a lower dipole will form more stably oriented liquids. All of the biopolymers can be dissolved in aqueous solvents to a high concentration and then processed and oriented as liquid crystals. However, the presence of solvent, the mid-range molar mass of the molecules, and our ability to engineer structural transitions into the sequences all contribute to the ability of these molecules to form solids which preserve liquid state order and orientation. Solidification is achieved through very accessible processes. In the solid state, the materials are thermally stable to over 100° C. and have reasonable mechanical properties as thin films. Key features affecting performance in the solid state include the stiffness of the molecule, e.g., whether alignment is retained or instead “relaxes” out; the size of the biopolymer needed to bind to and orient a chromophore, e.g., the chromophore may constitute as much of the composite composition as possible; and the optical clarity and physical properties of the solid as a thin film.

These materials can also be used in hydrogen catalysis. Hydrogen catalysis is a major component process in fuel cell development, an area which will grow in importance as the nation looks for ways to flexibly meet energy needs. This type of catalysis occurs at the surface of noble metal domains. To create a good reproducible catalyst, very small (nanoscale) reproducibly sized and shaped metallic nanodomains are necessary. A high surface area is expected to increase catalytic activity. Methods such as microlithography and chemical vapor deposition yield micron scale features, where far more metal is in the interior domain volume than at metal domain surfaces. Furthermore, noble metals, such as gold, are known to diffuse readily on smooth surfaces, limiting the size, density, and stability of patterns written. Conceivably, a nanolithographic technique, or “dip pen nanolithography” could be used to create a stable adsorbed chemical pattern which would hold the metal in place. This technique also suffers from the drawback of diffusion and spreading, in this case of the chemical pattern used to localize the noble metal. In both existing technologies, processes are used which create small high surface area metal patterns in a pattern-by-pattern or chip-by-chip manner. This impedes efficient scale up and makes manufacturing expensive. Major performance criteria include the size of metal domains, density of localized metal domains, reproducibility of metal domains and domain structure, and stability of metal domains against diffusion.

The self-fabricated materials, e.g., new polypeptides, can provide significant advantages over current technology. An amphiphilic “triblock” design with a rigid self-fabricating segment based loosley on spider or silkworm silk will provide performance advantages such as thermal and mechanical stability and chemical resistance as well as desirable pyrolysis behavior. Processed into a nanolayered ultrathin film, these molecules readily self-fabricate materials with 3-5 nanometer chemically alternating stripes exposed at the surface in spatially separate patterned domains, each having a very high density of stripes. These alternating stripes provide well-defined domains, which can adsorb and localize metal. The oligopeptide domains are formed through a process known as “thermodynamic frustration” which forces chemical subunits into structures that are compromizes between the different units (the “blocks” in the triblock) which give the best possible thermodynamic situation. The structures thus obtained depend simply on conditions such as temperature, concentration, pH, and molecule chemistry, and are thus highly reproducible. There is no stable state which we “work against” to process a pattern, rather we design a complex molecule and allow the energy to “roll downhill” to create very small chemically distinct stable patterns. Because the metal is chemically bound on a patterned chemically heterogeneous substrate, metal surface diffusion is inhibited, and very small densely packed lines of metal can be constructed.

Acid solubilizing blocks, affixed to both ends of a silk-like oligopeptide, have been observed to readily and reproducibly form nanostriped ultrathin thin films. The density and orientation of exposed stripes can be controlled by writing different chemically patterned variations in sequence into the silk-like self-fabricating block. A number of different bases and basic salts have been found to absorb into these acid terminated oligopeptides and are localized in the acid endblock layers (about 3 nm thick layers which alternate with 5-8 nm thick layers formed by the silk-like self-fabricating blocks). Since many of the noble metals will form compounds and salts with halides, metal halide will be adsorbed onto an ultrathin nanostriped film and “developed” to form a continuous domain of reduced metal. Similar “nanodeveloping” (as in a photographic emulsion) has been shown to work for nanowires adsorbed to DNA. In this case, pyrolysis of the composite oligopeptide metal halide material in a reducing gas (4% hydrogen, 96% argon) will be used to reduce the metal, while the charred oligopeptide will form a thermally stable matrix to immobilize the metal domains.

The self-fabricated materials beformed into ultrathin films by making a concentrated solution of the oligopeptide in trifluoroacetic acid or formic acid. Thin films are adsorbed from solution onto a hydrophobic surface such as hydrophobic ZnSe crystals, hydrophobic liquid interfaces with the aqueous oligopeptide solution, or the free surface made with the air. The thin films can be collected onto solid surfaces by skimming or dipping if they are not prepared by adsorption to a solid. The use of hydrophobic solid surfaces will also inhibit metal adsorption to exposed substrate areas if the films tear. The thin films are then treated by dipping them in solutions of metal halide to make nanocomposites, followed by pyrolization at 250° C. in a reducing gas atmosphere (4% hydrogen, 96% Argon) to obtain metallic nanodomains. The catalytic activity is then tested and the domain structures are characterized.

Because of the reversibility of the fabrication process, the polymers and the self-fabricated materials can also be used in automatic processes controlled by temperature. In addition, the self-fabricated materials are suitable to be used in cell culturing (especially in controlling the surface parameters) and implantation where cell adhesion on the surface of an implant can be controlled or prevented by a coating prepared with the material.

The miniblock polymers can also be used as biomaterials. For instance, they can be used as carriers for controlled release of drugs that are stored within layers. The miniblock polymers can also be used to form nanoporous in tissue mimetics, which favor the growth of particular cell types, by incorporating bound moieties known to inhibit bacterial growth in locations and patterns that maximize their effectiveness at low (safe) concentrations.

These miniblock polymers can also be used as novel optical materials with high order nonlinear optical properties (odd orders) in the visible and infrared regions, dispersive polarizing elements, attenuating gratings in the optical and infrared.

The miniblock polymers can also be used in nanolithographic processes, while significantly reducing the time and cost involved in a conventional lithographical process. Additional materials, such as optically active molecules, can be bound into rigid oriented portions of the miniblock polymer molecules, orienting the polymer molecules as well. The self-fabricated materials can also be used as coatings for biomaterials, scaffolds for tissue engineering, ferroelectric materials, piezoelectric materials, displays (e.g., liquid crystal applications), switching devices, artificial muscles and related biomaterials, actuators, membranes (e.g., for separation of enantiomers), and fuel cell catalyst substrates.

The invention is further described in the following examples, which are only illustrative and do not in any way limit the scope of the invention described in the claims.

### EXAMPLES

### Example 1

### Design of Peptide Sequences with Helix-Forming Blocks

Polypeptide sequences were designed to study the effects of sterics and to provide charged residues for patterning. More specifically, the following polypeptide sequences were designed to investigate the effect of containing a sterically bulky group on the dimension of a triple helix and on the subsequent self-assembly:

| | |
|---|---|
| (Glu)₅(Gly-Val-Pro-Gly-Pro-Val)₆(Glu)₅ | (SEQ ID NO:138) |
| (Glu)₅(Gly-Val-Pro-Gly-Pro-Ala)₆(Glu)₅ | (SEQ ID NO:139) |
| (Glu)₅(Gly-Leu-Pro-Gly-Pro-Pro)₆(Glu)₅ | (SEQ ID NO:140) |
| (Glu)₅(Gly-Ile-Pro-Gly-Pro-Pro)₆(Glu)₅ | (SEQ ID NO:141) |

All of these polypeptide sequences contained the amino acid residues valine, leucine, or isoleucine. These amino acid residues are all relatively large and are all hydrophobic, yet on different scales. None of these amino acid residues contain hetero atoms that would complicate comparison of the steric effects on triple helical conformation. Thus, these polypeptides represented a range of side chain sizes and a range of side chain hydrophobicity.

The following polypeptide sequences were designed to provide charge residues for patterning and to probe the possibility of using interfacial chemistry to strain triple helical conformation:

| | |
|---|---|
| (Lys)₅(Gly-Pro-Pro-Gly-Pro-Pro-Gly-Asp-Pro)₄(Lys)₅, | (SEQ ID NO:142) |
| and | |
| (Lys)₅(Gly-Ala-Pro-Gly-Pro-Pro-Gly-Asp-Pro)₄(Lys)₅. | (SEQ ID NO:143) |

Aspartic acid was chosen as an interaction handle because it is a small charged residue, and is a good hydrophilic handle which does not create a large sterical strain or disrupt the threefold helical structure. The combination of a nine-residue sequence periodicity and a ten residue helical periodicity resulted in patterns of aspartic acid residues, which were almost aligned along the helix, parallel to the helical axis. However, there was a small progressive angular offset or mismatch. In an interfacial experiment, there was a thermodynamic driving force to align these handle residues in the aqueous phase, resulting in straining, and in some cases deformation, of the triple helical conformation. Potential solubility problems of these polypeptides were circumvented by adding solubilizing lysine end blocks to the sequence.

In addition to the above-described polypeptide sequences, (G-S-P-G-P-P)ₙ (SEQ ID NO:144) and (G-A-G-A-G-S)ₙ (SEQ ID NO:145) were also designed for studies on the helical formation and assembling activities.

### Example 2

### Preparation of Polypeptides with Sequences Containing Helix-Forming Blocks

Polypeptides having the designed sequences shown above in Example 1 were produced to have a molecular weight of 15 kDa and 30 kDa using recombinant DNA techniques as described in, e.g., Prince et al., Biochem. 1995, 34, 10879; and Arcidiacono et al., Appl. Microbiol. Biotechnol. 1998, 49, 31-38. More specifically, repetitive oligonucleotide units were designed with codon choices optimized for expression and stability in E. coli. The oligonucleotides thus obtained were purified with high performance liquid chromatography (HPLC), constructed with 5′-NheI and 3′-SpeI termini, phosphorylated at 5′ ends using T4 polynucleotide kinase and complementary versions, and then annealed to form duplexes. A linker was previously constructed and ligated into pUC18 to facilitate cloning of these multimerized oligonucleotides. A plasmid containing the monomers was digested with NheI and combined with a second double digest (NheI and SpeI) to generate a plasmid-free insert. Multimers of different sizes were generated by repeated cycles and then removed from the recombinant pUC-LINK by digestion with BamHI, gel purified from 1% agarose, and then inserted into BamHI-digested and dephosphorylated pQE-9 or PET. E. coli SG13009pREP4 were transformed with the ligated mixture and successful transformants were determined by restriction digests for insert size and by Western assay on the expressed protein. The expressed proteins were purified by reverse phase HPLC using a C₁₈ column and a water/acetonitrile gradient with 0.1% TFA buffer mobile phase. The purified proteins were characterized for amino acid composition, N-terminal sequence and by laser adsorption.

### Example 3

### Preparation of Polypeptides with Sequences Containing Folding Blocks

Polypeptides having the designed sequences shown above in Example 1 were produced to have a molecular weight of 1 kDa and 12 kDa using recombinant DNA techniques as described in, e.g., Prince et al., Biochem. 1995, 34, 10879; and Arcidiacono et al., Appl. Microbiol. Biotechnol. 1998, 49, 31-38. More specifically, repetitive oligonucleotide units were designed with codon choices optimized for expression and stability in E. coli. The oligonucleotides thus obtained were purified with high performance liquid chromatography (HPLC), constructed with 5′-NheI and 3′-SpeI termini, phosphorylated at 5′ ends using T4 polynucleotide kinase and complementary versions, and then annealed to form duplexes. A linker was previously constructed and ligated into pUC18 to facilitate cloning of these multimerized oligonucleotides. A plasmid containing the monomers was digested with NheI and combined with a second double digest (NheI and SpeI) to generate a plasmid-free insert. Multimers of different sizes were generated by repeated cycles and then removed from the recombinant pUC-LINK by digestion with BamHI, gel purified from 1% agarose, and then inserted into BamHI-digested and dephosphorylated pQE-9 or PET. E. coli SG13009pREP4 were transformed with the ligated mixture and successful transformants were determined by restriction digests for insert size and by Western assay on the expressed protein. The expressed proteins were purified by reverse phase HPLC using a C₁₈ column and a water/acetonitrile gradient with 0.1% TFA buffer mobile phase. The purified proteins were characterized for amino acid composition, N-terminal sequence and by laser adsorption.

### Example 4

### Modeling Experiments

Effects of the amino acid sequence on the helical period of polypeptide triple helix were first determined by molecular mechanics modeling as described in Valluzzi et al., Macromolecules 1996, 29, 8606. In addition, the location and spacing of any supermolecular bending, bulging, or twisting was measured from energy minimized molecular models. Subsequently, a reliable estimate was made of the influence of polypeptide sequence on the setting angle of layers of the polypeptide triple helices before the sequences were synthesized. Large stiff residues were substituted to see the effect of residue substitution/sterics on the helical pitch and stability of the helix. A molecular mechanics minimization simulation, which utilized relatively little computer processor time, was used to generate sterically reasonable triple-helical structures for each modification in the sequence.

Constrained helices were also studied by using similar molecular mechanics simulations. In these helices, the sequences were conserved but the helical pitch was forced to vary. Local energy minima were obtained for the constrained structures and used to estimate the forces involved in altering the helical pitch and subsequent packing. Charged or polar residues were also used to study the effects of helix-helix recognition on packing in simulations involving several helices and possibly incorporating solvent molecules. Due to the large amount of processor time involved in such a simulation, a few systems of especially pressing interest were selected. A molecular dynamics simulation, where a system of several molecules was allowed to evolve over time, was designed to probe recognition behavior between helices. The results from the recognition simulation were used to design or refine sequences, choose the chemical environments for self-fabrication of collagen helices, and select interfacial systems to study membrane formation behavior.

### Example 5

### Interfacial Experiments

Polypeptides were placed at various liquid-liquid interfaces designed to interfere with the interhelical recognition to varying degrees, which in turn affects the pitch and packing. The studies focused on aqueous-organic solvent (liquid-liquid) and aqueous-air interfaces. Solvents were chosen to provide a range of interfacial energies, which affect the kinetics of interfacial film formation. Various solvents were used to probe the interaction between the organic solvent phase and specific residues in the polypeptide, and the subsequent influence of this interaction on conformation and self-fabrication. The solvents included n-butanol, hexane, octane, chloroform, and dioxane. Dioxane and n-butanol are more soluble in water (but still only slightly soluble) than hexane, octane, and chloroform, and therefore produced interfaces with lower surface energies. Chloroform contains a polarizable heteroatom, which might influence the van der Waals interactions between the solvent and the polypeptide. Hexane and octane were non-polar hydrocarbons which formed interfaces with water that were similar in energy. Differences observed in interfacial protein films prepared with these two solvents were attributable to the organic solvent size and packing at the interface.

Air-Water Interface:

Protein samples were prepared from dry powders of purified proteins. More specifically, the proteins were dissolved in formic acid, acetic acid, or aqueous ammonium acetate salt solution. The resultant solutions were dialyzed against frequently changed distilled water for one week using a 10,000 to 12,000 molecular weight cut off dialysis membrane to remove the acid or salt. As a control, another series of experiments were performed with the protein in dilute acid solution. Since ammonium acetate and formic anhydride were reasonably volatile, extensive dialysis was not always necessary. The protein solution was pipetted onto the surface of a Langmuir trough (Lauda) containing an aqueous subphase for a final concentration of protein of around 0.1%. The films that form were deposited onto TEM grids using standard dipping techniques. Samples from the surface of the Langmuir trough were taken over time at different surface pressures. Uncompressed films were also prepared as a baseline for studies of concentration, pressure and other trough conditions on the orientation and structure of the resulting films.

Aqueous-Organic Liquid-Liquid Interface:

For aqueous-organic solvent studies, the aqueous fibroin and recombinant protein solutions (prepared as above) were placed in vials and covered with HPLC grade solvent (e.g., hexane). The vials were sealed with airtight caps to reduce evaporation. Interfacial films were then collected on TEM grids at intervals over time. A number solvents were used to probe different possible influences on conformation selection and aggregation. A series of alkanes were used to form the interface with the aqueous collagen solutions. Some additional solvents, such as chlorinated solvents, incorporating varying polarities, were also used. The chemistry of the aqueous phase was also varied. In all cases, parallel experiments were run at concentrations of collagen or synthetic polypeptides ranging from 0.1 to 5 wt %. More concentrated solutions were also studied whenever the solubility of the polypeptides permits. For each combination of a solvent and a concentration of protein, several sealed interfacial systems were prepared for studies on the evolution of the interfacial films over time while always sampling fresh, undisturbed interfaces. Standard protein crystallization techniques and reagents (e.g., surfactants, salts) were used with the polypeptide solutions in order to determine which of the interfacial structures can be reproduced in bulk and under what conditions. By comparing the interfacial and bulk conditions that give rise to the same crystal structure, one can determine which interactions are most important to conformation selection and aggregation.

### Example 6

### Preparation of Self-Fabricated Materials

Purified polypeptides obtained from Example 2 were dissolved in an aqueous solution or alcohol at concentrations of 40 to 500 mg/mL. The solutions were placed in a dry environment of constant temperature at 0-5° C. After 8-36 hours, liquid crystalline materials started to form and were observed with polarizing optical microscopy. They were collected in dried form as flakes or films which were easily removed from the container with tweezers.

### Example 7

### Characterization of Polymers and Self-Fabricated Materials

For a meaningful interpretation of the effects of interfacial chemistry or bulk chemistry and processing conditions on the subsequent aggregation and self-fabrication behavior of collagens, characterization of any aggregates that might exist in bulk and at interfaces was necessary.

Analysis of Samples from Bulk Experiments Circular Dichroism (CD)

To determine the triple helical tendencies of the synthetic poylpeptides, CD spectra in the far UV, 190 to 240 nm range were obtained on a Jasco Spectrophotometer with a 10 mm path length cell by methods described in Prince, J. T. et al., Biochem. 1995, 34, 10879. Secondary structure was analyzed using algorithms provided by the manufacturer.

Analysis of Samples Formed at Interfaces

Transmission Electron Microscopy (TEM), and Electron Diffraction (ED)

Crystal morphology and crystal structures in the films were determined by TEM and ED with a JEOL 2000FX TEM operated at 200 kV. Additionally, thicker films were scanning transmission electron microscopy (STEM) to ensure that the morphologies were comparable to those observed in the thinner films. Elemental analysis was conducted using light element PEELS and XPS to ensure that the regions studied have an elemental composition appropriate to a protein or polypeptide. An internal gold standard was used to determine lattice spacing. Low dosage was required to minimize beam damage and the cryogenic temperatures were around −160° C. since proteins are very beam-sensitive. Pairs of diffraction patterns will be taken at two different tilt orientations between the samples and incident beam in order to determine the crystalline orientation in the films. Additionally film morphology and the structure of crystals templated on the polypeptide films were examined using high-resolution field emission SEM.

Atomic Force Microscopy (AFM)

AFM was carried out on interfacial films and deposited materials in tapping mode on a Digital Nanoscope III system.

X-Ray Diffraction Analysis (XRD)

XRD was performed on an Elliot GX-20 rotating anode run at 35 kV by 25 mA. A Frank's camera was used with a 200-mm spot and a specimen to film distance of 74.2 mm calibration with calcite.

### Example 8

### Fourier Transform Infrared Spectroscopy (FTIR) Studies FTIR attenuated total reflectance (ATR) spectra were obtained on a Bruker Infrared Spectrophotometer. Germanium prisms (Harrick Model EJ3 122, 50×102 mm, spp 45°) were used for the polypeptide films. The amide A band for triple helical model polypeptides was correlated with superhelical coiling¹.

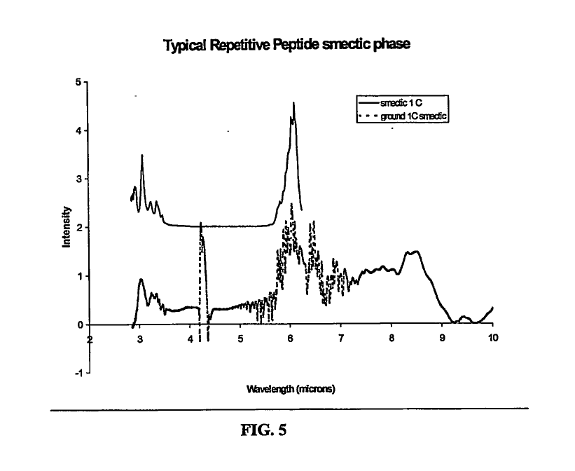

As demonstrated in FIGS. 4 and 5, the tested polypeptides had activities throughout the FTIR spectrum. In FIG. 4, the upper curve corresponds to the IR absorbance of a self-assembled material at 1° C., while the lower curve corresponds to the IR absorbance of the polypeptides after the material was ground to destroy its long-range order (i.e., the self-assembly of polymers was disrupted). In other words, self-assembly of the polypeptides resulted in higher IR absorption, and, when the resulting self-assembled material was mechanically ground to reduce the long range supermolecular ordering, FTIR behavior typical of large molecules began to re-emerge.

At 1° C., the repetitive collagen-like peptides formed chiral smectic phases with twist (supermolecular helix) wavelengths of 3-5 microns, which corresponded to a region in the FTIR where the expected FTIR activities can no longer be observed. This change became more apparent as more of the chiral smectic superstructure was destroyed by grinding. This effect was observed in both reflection and transmission modes.

FIG. 5 shows that polypeptides of less repetitive sequences respond to FTIR attenuation in a region of shorter wavelengths 2.7-4.25 microns. For these peptides, a number of strong FTIR absorbance wavelengths typically occur in the 34 micron region which are undetectable for this peptide when it is organized in the smectic state. The combination of the transmission and reflection FTIR spectra indicates that the IR response properties are of the materials, not just on their surfaces.

The spectral differences between the materials assembled from the repetitive and less repetitive polypeptides suggest that a mechanism for FTIR manipulation by these polypeptide materials can be isolated and used to target particular material features leading to desirable FTIR properties. These materials features can then be engineered into the structures at the molecular level; and the periodicity of the chiral length scales in the materials can be tuned through the choice of repetitive sequences.

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the claims.

1. A miniblock polymer comprising a self-fabricating block and a solubilizing block, wherein the miniblock polymer has a molecular weight of about 1,000 to about 300,000 and, in solution, can self-fabricate to form a three-dimensional material having a long-range order, and wherein the miniblock polymer has a glycine content of at least 20%.

2. The miniblock polymer of claim 1, wherein the self-fabricating block causes the miniblock polymer to form a helix.

3. The miniblock polymer of claim 2, wherein the miniblock polymer comprises two solubilizing blocks as end blocks, and the self-fabricating block as a core block.

4. The miniblock polymer of claim 2, wherein the self-fabricating block contains glycine at every third position of the sequence of the block.

5. The miniblock polymer of claim 1, wherein the miniblock polymer is a polypeptide.

6. The miniblock polymer of claim 1, wherein the miniblock polymer comprises at least two solubilizing blocks and one self-fabricating block.

7. The miniblock polymer of claim 6, wherein the self-fabricating block has a glycine content of 30-80%.

8. The miniblock polymer of claim 1, further comprising one or more blocks for triggering self-fabrication by external or environmental conditions.

9. The miniblock polymer of claim 8, wherein the external or environmental condition is selected from the group consisting of temperature, light, redox chemistry, enzymatic reactions, and pH.

10. The miniblock polymer of claim 8, wherein the self-fabricating block causes the miniblock polymer to form a helix.

11. The miniblock polymer of claim 9, wherein the miniblock polymer comprises two solubilizing blocks as end blocks, and the self-fabricating block is within the core block.

12. The miniblock polymer of claim 9, wherein the self-fabricating block contains glycine at every third position of the sequence of the block.

13. The miniblock polymer of claim 8, wherein the miniblock polymer is a polypeptide.

14. The miniblock polymer of claim 8, wherein the miniblock polymer comprises at least two solubilizing blocks and one self-fabricating block.

15. The miniblock polymer of claim 13, wherein the self-fabricating block has a glycine content of 30-80%.

16. The miniblock polymer of claim 1, further comprising a block for incorporating turns in the polymer or for providing sites for chemical modifications.

17. The miniblock polymer of claim 16, wherein the self-fabricating block causes the miniblock polymer to form a helix.

18. The miniblock polymer of claim 17, wherein the miniblock polymer comprises two solubilizing blocks as end blocks, and the self-fabricating block is within the core block.

19. The miniblock polymer of claim 17, wherein the self-fabricating block contains glycine at every third position of the sequence of the block.

20. The miniblock polymer of claim 16, wherein the miniblock polymer is a polypeptide.

21. The miniblock polymer of claim 16, wherein the miniblock polymer comprises at least two solubilizing blocks and one self-fabricating block.

22. The miniblock polymer of claim 21, wherein the self-fabricating block has a glycine content of 30-80%.

23. The miniblock polymer of claim 1, wherein the self-fabricating block contains glycine every third monomer, contains greater than 25% amino acids, and has a total block length of more than 6 monomers.

24. The miniblock polymer of claim 1, wherein the self-fabricating block undergoes a random coil to helix transition.

25. The miniblock polymer of claim 1, wherein the self-fabricating block is a confirmation selecting block imparting either hydrophobicity or hydrophilicity to the overall miniblock polymer and stabilizing secondary structure intermediates between a twofold extended helix and a threefold extended helix.

26. The miniblock polymer of claim 1, wherein the self-fabricating block contains native sequences found in spider silks, prions, or amyloids.

27. The miniblock polymer of claim 1, wherein the glycine exists in a glycine block selected from the group consisting of (GX)ₙ (SEQ ID NO: 146); (GGX)ₙ (SEQ ID NO: 147), (XG)ₙ (SEQ ID NO: 148), (xGG)ₙ (SEQ ID NO: 149), (GXX)N (SEQ ID NO: 150), (XXG)ₙ (SEQ ID NO: 151), (GGXGXX)ₙ (SEQ ID NO: 152), (GXGXGGXGXX)ₙ (SEQ ID NO: 153), and their combinations, wherein X is a non-glycine amino acid, and n is an integer from 1 to 5 inclusive.

28. The miniblock polymer of claim 1, wherein the glycine exists in a glycine block selected from the group consisting of GAGAGS (SEQ ID NO: 7), GAGAGY (SEQ ID NO: 8), and (G[A₂V₂L₂I]G[A₂V₂L₂I])ₙG[Y₂S₂T]) (SEQ ID NO: 9), wherein n is an integer from 1 to 5 inclusive.

29. The miniblock polymer of claim 1, wherein the self-fabricating block has a peptide sequence selected from the group consisting of:

| | |
|---|---|
| GGAGGGGTGGLGSGGAGAGGLGGGGAG; | (SEQ ID NO:10) |
| GDVGGAGATGGS; | (SEQ ID NO:11) |
| GNVGGAGASGGS; | (SEQ ID NO:12) |
| GAIGGVGATGGS; | (SEQ ID NO:13) |
| SGAGVGRGDGSGVGLGSGNG; | (SEQ ID NO:14) |
| GPGGTGPGQQGPGGTW; | (SEQ ID NO:15) |
| GPGNNGPGGYGPGNNGPSGPGSA; | (SEQ ID NO:16) |
| DPGVYGPSGNAPGVYGPSGQGAGAGS; | (SEQ ID NO:17) |
| GVGVGS; | (SEQ ID NO:18) |
| GIGIGS; | (SEQ ID NO:19) |
| GVGGGY; | (SEQ ID NO:20) |
| GAGAGY; | (SEQ ID NO:21) |
| GAGAGD; | (SEQ ID NO:22) |
| SGRGGLGGQGAGGGAGQGGYGGLGSQG; | (SEQ ID NO:23) |
| MKHMAGAAGAVVGGLGGYMLGSAM; | (SEQ ID NO:24) and |
| GAGAGT. | (SEQ ID NO:25) |

30. The miniblock polymer of claim 1 having a peptide sequence selected from the group consisting of:

| | |
|---|---|
| (E)₅G (C)₂(GAPGPP)₅(C)₂G(E)₅; | (SEQ ID NO:34) |
| (E)₅ GCCE (GAPGPP)₂ GAPGPR-GDPGPP-GAPGPP(C)₂ G (E)₅; | (SEQ ID NO:35) |
| (E)₂CERGDE (GAPGPP)₅ERGDEC (E)_{2;} | (SEQ ID NO:36) |
| (E)₅ (GAPGPP)₂ GCPGPP (GAPGPP)₂ (E)₅; | (SEQ ID NO:37) |
| (E)₂G (C)₄(GAPGPP)₅(C)₄G (E)_{2;} | (SEQ ID NO:38) |
| (E)₂G (C)₄(GVPGPP)₅(C)₄G (E)_{2;} | (SEQ ID NO:39) |
| (E)₃(GCPGPC)₆(E)₃; | (SEQ ID NO:40) |
| (E)₃(GPAGPP)₄(E)₃; | (SEQ ID NO:41) |
| (E)₃(GA⊖PGP⊖P)₄(E)₃; | (SEQ ID NO:42) |
| (E)₃(GV⊖PGP⊖P)₄(E)₃; | (SEQ ID NO:43) |
| (E)₅(GPPGVP-GPPGPS-GPPGVP-GSPGPP-GPVGPS-GPP)(E)₅; | (SEQ ID NO:44) |
| (E)₅(GAPGP⊖P)₆(E)₅; | (SEQ ID NO:45) |
| (E)₅(GV⊖PGP⊖P)₆(E)₅; | (SEQ ID NO:46) |
| (K)₅(GAPGPPGDP)₄(K)₅; | (SEQ ID NO:47) |
| N₅ (GPAGPP)₆ N₅; | (SEQ ID NO:48) |
| N₅ (GAPGPP)₆ N₅; | (SEQ ID NO:49) |
| N₅(GPVGPP)₆N₅; | (SEQ ID NO:50) |
| N₅(GVPGPP)₆N₅; | (SEQ ID NO:51) |
| NGSNN(GAPGPP)₆ NGSNN; | (SEQ ID NO:52) |
| N₅(GPAGPP)₃GPRGDP(GAPGPP)₃N₅; | (SEQ ID NO:53) |
| (E)₅(GVPGPV)₆(E)₅; | (SEQ ID NO:54) |
| (E)₅(GVPGPA)₆(E)₅; | (SEQ ID NO:55) |
| (GAPGPP)ₙ; | (SEQ ID NO:56) |
| (E)₅(GVPGPP)₆(E)₅; | (SEQ ID NO:57) |
| (E)₅(GLPGPP)₆(E)₅; | (SEQ ID NO:58) |
| (E)₅(GLPGPP)₆(E)₅; | (SEQ ID NO:59) and |
| (K)₅(GPPGPPGDP)₄(K)₅. | (SEQ ID NO:60) |

31. The miniblock polymer of claim 1 having a peptide sequence selected from the group consisting of:

| | |
|---|---|
| SAASAASAASAASAASAA; | (SEQ ID NO:61) |
| SALSVASIASALSVASIA; | (SEQ ID NO:62) |
| YALSVATIAYALSVATIA; | (SEQ ID NO:63) |
| AAAAYAAAAYAAAAYAAAAY; | (SEQ ID NO:64) |
| AAAAYAAYAAYAAYAAY; | (SEQ ID NO:65) |
| AAASSIIIAAASIIIAAASS; | (SEQ ID NO:66) |
| (E)₃(A)₁₅(E)₃; | (SEQ ID NO:67) |
| (E)₃(A)₂₀(E)₃; | (SEQ ID NO:68) |
| (E)₃(A)₂₂(E)₃; | (SEQ ID NO:69) |
| (EAAAK)₄; | (SEQ ID NO:70) and |
| (EAAAK)₅. | (SEQ ID NO:71) |

32. The miniblock polymer of claim 1 having a peptide sequence selected from the group consisting of:

| | |
|---|---|
| GIGIGS; | (SEQ ID NO:72) |
| (E)₅(GAGAGD)₄(E)₅; | (SEQ ID NO:73) |
| (E)₅(GVGVGD)₄(E)₅; | (SEQ ID NO:74) |
| (E)₅(GLGLGD)₄(E)₅; | (SEQ ID NO:75) |
| (E)₅(GIGIGD)₄(E)₅; | (SEQ ID NO:76) |
| (E)₅(GVGVGY)₄(E)₅; | (SEQ ID NO:77) |
| (E)₅(GKGAGD)₄(E)₅; | (SEQ ID NO:78) |
| (E)₅(GAGAGT)₄(E)₅; | (SEQ ID NO:79) |
| (E)₅(GGAGGA)₄(E)₅; | (SEQ ID NO:80) |
| (E)₅(GGAGGT)₄(E)₅; | (SEQ ID NO:81) |
| (E)₅(GGAGGD)₄(E)₅; | (SEQ ID NO:82) |
| SGAGVGRGDGSGVGLGSGNG; | (SEQ ID NO:83) |
| GDVGGAGATGGS; | (SEQ ID NO:84) |
| GNVGGAGASGGS; | (SEQ ID NO:85) |
| GGAGGGGTGGLGSGG; | (SEQ ID NO:86) |
| GGAGGGGTGGLGSGGAGAGGLGGGGAG; | (SEQ ID NO:87) |
| GPGGTGPGQQGPGGTW; | (SEQ ID NO:88) |
| GPGNNGPGGYGPGNNGPSGPGSA; | (SEQ ID NO:89) |
| DPGVYGPSGNAPGVYGPSGQGAGAGS; | (SEQ ID NO:90) |
| (E)₅(GAGAGS)₄(E)₅; | (SEQ ID NO:91) |
| (E)₅(GVGVGS)₄(E)₅; | (SEQ ID NO:92) |
| (E)₅(GIGIGS)₄(E)₅; | (SEQ ID NO:93) |
| (E)₅(GVGVGY)₄(E)₅; | (SEQ ID NO:94) |
| (E)₅(GAGAGY)₄(E)₅; | (SEQ ID NO:95) |
| (E)₅(GAGAGD)₄(E)₅; | (SEQ ID NO:96) |
| (E)₄(GAGAGS)₂(E)₄(GAGAGS)(E)₄; | (SEQ ID NO:97) |
| (E)₄(GAGAGS)(E)₄(GAGAGS)(E)₄; | (SEQ ID NO:98) |
| (E)₆(GAGAGD)₃(E)₆; | (SEQ ID NO:99) |
| (E)₆(GVGVGS)₃(E)₆; | (SEQ ID NO:100) |
| (E)₆(GIGIGS)₃(E)₆; | (SEQ ID NO:101) |
| (E)₆(GVGVGY)₃(E)₆; | (SEQ ID NO:102) |
| (E)₆(GAGAGS)₃(E)₆; | (SEQ ID NO:103) |
| (E)₅(GPGGYGPGQQGPGGY)(E)₅; | (SEQ ID NO:104) |
| (E)₅(GPGQQGPGGYGPGQQGPSGPGSA)(E)₅; | (SEQ ID NO:105) |
| (E)₅(DPGVY-GPSGQ-DPGVY-GPSGQ)(E)₅; | (SEQ ID NO:106) |
| GAIGGVGATGGS; | (SEQ ID NO:107) |
| (R)₃(GGAGQGGYGGLGSQGAGRGGLGGQGAG)(R)₃; | (SEQ ID NO:108) |
| GVGVGS; | (SEQ ID NO:109) |
| (E)₅(SGAGVG-RGDGSGV-GLGSGNG)₂(E)₅; | (SEQ ID NO:110) |
| (E)₅(GDIGGV-GATGGS)₂(E)₅; | (SEQ ID NO:111) |
| (E)₅(GNVGGA GASGGS)₂(E)₅; | (SEQ ID NO:112) |
| (E)₅(GVDGGA-GATGGS)₂(E)₅; | (SEQ ID NO:113) |
| GVGGGY | (SEQ ID NO:114) |
| GAGAGY; | (SEQ ID NO:115) |
| GAGAGD; | (SEQ ID NO:116) |
| SGRGGLGGQGAGGGAGQGGYGGLGSQG; | (SEQ ID NO:117) |
| GAGAGT; | (SEQ ID NO:118) |
| (SGAGVGRGDGSGVGLGSGNG); | (SEQ ID NO:119) |
| (R)₃(GGAGQGGYGGLGSQGAGRGGLGGQGAG)(R)₃; | (SEQ ID NO:120) |
| (E)₅(GDVGGAGATGGS)₂(E)₅; | (SEQ ID NO:121) |
| (E)₅(GDVGGAGASGGS)₂(E)₅; | (SEQ ID NO:122) |
| (E)₅(GDIGGVGATGGS)₂(E)₅; | (SEQ ID NO:123) |
| (E)₅(GPGGYGPGQQGPGGY)(E)₅; | (SEQ ID NO:124) |
| (E)₅(GPGQQ-GPGGY-GPGQQ-GPSGPG-SA)(E)₅; | (SEQ ID NO:125) and |
| (E)₅(DPGVY-GPSGQ-DPGVY-GPSGQ)(E)₅. | (SEQ ID NO:126) |

33. The miniblock polymer of claim 1 having a peptide sequence selected from the group consisting of:

| | |
|---|---|
| SGRGGLGGQGAGMAAAAAMGGAGQGGYGGLGSQG; | (SEQ ID NO:127) |
| MKHMAGAAGAVVGGLGGYMLGSAM; | (SEQ ID NO:128) |
| MDAEFRHDSGYEVHHQKLVFFAEDVGSNKGAIIGLMVGGVVIATVIVITLVM; | (SEQ ID NO:129) and |
| (E)₄(VSSTGSTSNTDSSSKSAGSRTSGGTSTYGYSSSHRGGS)(E)₄. | (SEQ ID NO:130) |

34. The miniblock polymer of claim 1, wherein the miniblock polymer contains a peptide sequence selected from the group consisting of:
DEDEDED (SEQ ID NO: 131);
GAGAGSGAGAGSGAGAGS (SEQ ID NO: 132);
GAPGPP (SEQ ID NO: 133);
GPAGPP (SEQ ID NO: 134);
GPAGPP (SEQ ID NO: 135);
GAPGPP (SEQ ID NO: 136); and combinations thereof.

35. The miniblock polymer of claim 1 having the peptide sequence:

| | |
|---|---|
| (EDE)₅(GAGAGS)₄RGDS(GAGAGS)₄PGP(GAGAGY)₂PGP(GAGAGS)₂(EDE)₄. | (SEQ ID NO:32) |

36. The miniblock polymer of claim 1 having the peptide sequence:
(SEQ ID NO:158)
NYNS (GCCCG)₃ NSYS (GPGGTGPGQQGPGGTW)₂
GPG (GVGVGSGAGAGD)₃.

37. The miniblock polymer of claim 1 having the peptide sequence:
(SEQ ID NO:159)
GEGP MKHMA EGPG (GAGAGYGAGY) GPG GESYRGDGSG.

38. The miniblock polymer of claim 1 having the peptide sequence:

| | |
|---|---|
| GPG (GVPGVAGGP)₃ SYSSNR. | (SEQ ID NO:160) |

39. The miniblock polymer of claim 1 having the peptide sequence selected from the group consisting of:
(E)₅(GVPGPV)₆(E)₅ (SEQ ID NO: 54);
(E)₅(GVPGPA)₆(E)₅ (SEQ ID NO: 55);
(E)₅(GLPGPP)₆(E)₅ (SEQ ID NO: 58);
(E)₅(GIPGPP)₆(E)s (SEQ ID NO: 59);
(K)₅(GPPGPPGDP)₄(K)₅ (SEQ ID NO: 60);
(K)₅(GAPGPPGDP)₄(K)₅ (SEQ ID NO: 47);
(GSPGPP)N (SEQ ID NO: 154); and
(GAGAGS)N (SEQ ID NO: 155); wherein n is an integer from 1 to 5 inclusive.

40. A miniblock polymer comprising a self-fabricating block, a solubilizing block, a block for triggering self-fabrication by external or environmental conditions, and a block for incorporating turns in the polymer or for providing sites for chemical modifications, wherein the miniblock polymer has a molecular weight of about 1,000 to about 300,000 and, in solution, can self-fabricate to form a three-dimensional material having a long-range order, and wherein the miniblock polymer has a glycine content of at least 20%.

41. The miniblock polymer of claim 40, wherein the self-fabricating block causes the miniblock polymer to form a helix.

42. The miniblock polymer of claim 41, wherein the miniblock polymer comprises two solubilizing blocks as end blocks, and the self-fabricating block is within the core block.

43. The miniblock polymer of claim 41, wherein the self-fabricating block contains glycine at every third position of the sequence of the block.

44. The miniblock polymer of claim 40, wherein the miniblock polymer is a polypeptide.

45. The miniblock polymer of claim 40, wherein the miniblock polymer comprises at least two solubilizing blocks and one self-fabricating block.

46. The miniblock polymer of claim 45, wherein the self-fabricating block has a glycine content of 30-80%.

47. A self-fabricated material having a three-dimensional structure, comprising a plurality of the miniblock polymers of claim 1, 8, 16, or 40.

48. The self-fabricated material of claim 47, wherein the self-fabricated material has on its surface a patterned film of inorganic, organometallic, or optically active material.

49. A method of controlled delivery of a drug, the method comprising incorporating a drug within the self-fabricated material of claim 1, 8, 16, or 40, and administering to a subject in need thereof the self-fabricating material incorporating the drug.

50. The method of claim 49, wherein the drug is incorporated within layers of the self-fabricating material.

51. A method of modifying the optical response of a device in the near to mid infrared wavelength range, the method comprising applying a self-fabricated material of claim 47 to the surface of the device.

52. A miniblock polymer comprising multimeric associated helical segments and non-helical segments, wherein the polymer in solution can self-assemble to form a material having a long-range order and at least one of the helical segments comprises a peptide sequence having a glycine (G) residue at every third position.

53. The polymer of claim 52, wherein the non-helical segments are hydrophobic.

54. The polymer of claim 52, wherein the non-helical segments comprise glutamic acid.

55. A self-fabricated material having a long-range order in the solid glassy state, wherein the material is prepared from a miniblock polymer comprising helical and non-helical segments.


SEQUENCE LISTING (163 sequences)

SEQ ID NO: 1 - protein, 50 aa, Artificial Sequence
  Description of Artificial Sequence Synthetic peptide
EEEEESGAGVGRGDGSGVGLGSGNGSGAGVGRGDGSGVGLGSGNGEEEEE

SEQ ID NO: 2 - protein, 6 aa, Artificial Sequence
  Description of Artificial Sequence Synthetic peptide
GGXGXX

SEQ ID NO: 3 - protein, 10 aa, Artificial Sequence
  Description of Artificial Sequence Synthetic peptide
GXGXGGXGXX

SEQ ID NO: 4 - protein, 5 aa, Artificial Sequence
  Description of Artificial Sequence Synthetic peptide
GXGGX

SEQ ID NO: 5 - protein, 7 aa, Artificial Sequence
  Description of Artificial Sequence Synthetic peptide
GXXAXXG

SEQ ID NO: 6 - protein, 10 aa, Artificial Sequence
  Description of Artificial Sequence Synthetic peptide
GGXGGGXGXX

SEQ ID NO: 7 - protein, 6 aa, Artificial Sequence
  Description of Artificial Sequence Synthetic peptide
GAGAGS

SEQ ID NO: 8 - protein, 6 aa, Artificial Sequence
  Description of Artificial Sequence Synthetic peptide
GAGAGY

SEQ ID NO: 9 - protein, 22 aa, Artificial Sequence
  Description of Artificial Sequence Synthetic peptide
GXGXGXGXGXGXGXGXGXGXGX

SEQ ID NO: 10 - protein, 27 aa, Artificial Sequence
  Description of Artificial Sequence Synthetic peptide
GGAGGGGTGGLGSGGAGAGGLGGGGAG

SEQ ID NO: 11 - protein, 12 aa, Artificial Sequence
  Description of Artificial Sequence Synthetic peptide
GDVGGAGATGGS

SEQ ID NO: 12 - protein, 12 aa, Artificial Sequence
  Description of Artificial Sequence Synthetic peptide
GNVGGAGASGGS

SEQ ID NO: 13 - protein, 12 aa, Artificial Sequence
  Description of Artificial Sequence Synthetic peptide
GAIGGVGATGGS

SEQ ID NO: 14 - protein, 20 aa, Artificial Sequence
  Description of Artificial Sequence Synthetic peptide
SGAGVGRGDGSGVGLGSGNG

SEQ ID NO: 15 - protein, 16 aa, Artificial Sequence
  Description of Artificial Sequence Synthetic peptide
GPGGTGPGQQGPGGTW

SEQ ID NO: 16 - protein, 23 aa, Artificial Sequence
  Description of Artificial Sequence Synthetic peptide
GPGNNGPGGYGPGNNGPSGPGSA

SEQ ID NO: 17 - protein, 26 aa, Artificial Sequence
  Description of Artificial Sequence Synthetic peptide
DPGVYGPSGNAPGVYGPSGQGAGAGS

SEQ ID NO: 18 - protein, 6 aa, Artificial Sequence
  Description of Artificial Sequence Synthetic peptide
GVGVGS

SEQ ID NO: 19 - protein, 6 aa, Artificial Sequence
  Description of Artificial Sequence Synthetic peptide
GIGIGS

SEQ ID NO: 20 - protein, 6 aa, Artificial Sequence
  Description of Artificial Sequence Synthetic peptide
GVGGGY

SEQ ID NO: 21 - protein, 6 aa, Artificial Sequence
  Description of Artificial Sequence Synthetic peptide
GAGAGY

SEQ ID NO: 22 - protein, 6 aa, Artificial Sequence
  Description of Artificial Sequence Synthetic peptide
GAGAGD

SEQ ID NO: 23 - protein, 27 aa, Artificial Sequence
  Description of Artificial Sequence Synthetic peptide
SGRGGLGGQGAGGGAGQGGYGGLGSQG

SEQ ID NO: 24 - protein, 24 aa, Artificial Sequence
  Description of Artificial Sequence Synthetic peptide
MKHMAGAAGAVVGGLGGYMLGSAM

SEQ ID NO: 25 - protein, 6 aa, Artificial Sequence
  Description of Artificial Sequence Synthetic peptide
GAGAGT

SEQ ID NO: 26 - protein, 6 aa, Artificial Sequence
  Description of Artificial Sequence Synthetic peptide
GVPGPV

SEQ ID NO: 27 - protein, 6 aa, Artificial Sequence
  Description of Artificial Sequence Synthetic peptide
GVPGPA

SEQ ID NO: 28 - protein, 6 aa, Artificial Sequence
  Description of Artificial Sequence Synthetic peptide
GLPGPP

SEQ ID NO: 29 - protein, 6 aa, Artificial Sequence
  Description of Artificial Sequence Synthetic peptide
GIPGPP

SEQ ID NO: 30 - protein, 9 aa, Artificial Sequence
  Description of Artificial Sequence Synthetic peptide
GPPGPPGAP

SEQ ID NO: 31 - protein, 9 aa, Artificial Sequence
  Description of Artificial Sequence Synthetic peptide
GAPGPPGAP

SEQ ID NO: 32 - protein, 109 aa, Artificial Sequence
  Description of Artificial Sequence Synthetic peptide
EDEEDEEDEEDEEDEGAGAGSGAGAGSGAGAGSGAGAGSRGDSGAGAGSGAGAGSGAGAGSGAGAGSPGPGAGAGYGAGAGYPGPGAGAGSGAGAGSEDEEDEEDEEDE

SEQ ID NO: 33 - protein, 166 aa, Artificial Sequence
  Description of Artificial Sequence Synthetic peptide
NYNSGCCCGGCCCGGCCCGNSYSGPGGTGPGQQGPGGTWGPGGTGPGQQGPGGTWGPGGVGVGSGAGAGDGVGVGSGAGAGDGVGVGSGAGAGDGEGPMKHMAEGPGGAGAGYGAGYGPGGESYRGDGSGGPGGVPGVAGGPGVPGVAGGPGVPGVAGGPSYSSNR

SEQ ID NO: 34 - protein, 46 aa, Artificial Sequence
  Description of Artificial Sequence Synthetic peptide
EEEEEGCCGAPGPPGAPGPPGAPGPPGAPGPPGAPGPPCCGEEEEE

SEQ ID NO: 35 - protein, 47 aa, Artificial Sequence
  Description of Artificial Sequence Synthetic peptide
EEEEEGCCEGAPGPPGAPGPPGAPGPRGDPGPPGAPGPPCCGEEEEE

SEQ ID NO: 36 - protein, 46 aa, Artificial Sequence
  Description of Artificial Sequence Synthetic peptide
EECERGDEGAPGPPGAPGPPGAPGPPGAPGPPGAPGPPERGDECEE

SEQ ID NO: 37 - protein, 40 aa, Artificial Sequence
  Description of Artificial Sequence Synthetic peptide
EEEEEGAPGPPGAPGPPGCPGPPGAPGPPGAPGPPEEEEE

SEQ ID NO: 38 - protein, 44 aa, Artificial Sequence
  Description of Artificial Sequence Synthetic peptide
EEGCCCCGAPGPPGAPGPPGAPGPPGAPGPPGAPGPPCCCCGEE

SEQ ID NO: 39 - protein, 44 aa, Artificial Sequence
  Description of Artificial Sequence Synthetic peptide
EEGCCCCGVPGPPGVPGPPGVPGPPGVPGPPGVPGPPCCCCGEE

SEQ ID NO: 40 - protein, 42 aa, Artificial Sequence
  Description of Artificial Sequence Synthetic peptide
EEEGCPGPCGCPGPCGCPGPCGCPGPCGCPGPCGCPGPCEEE

SEQ ID NO: 41 - protein, 30 aa, Artificial Sequence
  Description of Artificial Sequence Synthetic peptide
EEEGPAGPPGPAGPPGPAGPPGPAGPPEEE

SEQ ID NO: 42 - protein, 30 aa, Artificial Sequence
  Description of Artificial Sequence Synthetic peptide
EEEGAPGPPGAPGPPGAPGPPGAPGPPEEE

SEQ ID NO: 43 - protein, 30 aa, Artificial Sequence
  Description of Artificial Sequence Synthetic peptide
EEEGVPGPPGVPGPPGVPGPPGVPGPPEEE

SEQ ID NO: 44 - protein, 43 aa, Artificial Sequence
  Description of Artificial Sequence Synthetic peptide
EEEEEGPPGVPGPPGPSGPPGVPGSPGPPGPVGPSGPPEEEEE

SEQ ID NO: 45 - protein, 46 aa, Artificial Sequence
  Description of Artificial Sequence Synthetic peptide
EEEEEGAPGPPGAPGPPGAPGPPGAPGPPGAPGPPGAPGPPEEEEE

SEQ ID NO: 46 - protein, 46 aa, Artificial Sequence
  Description of Artificial Sequence Synthetic peptide
EEEEEGVPGPPGVPGPPGVPGPPGVPGPPGVPGPPGVPGPPEEEEE

SEQ ID NO: 47 - protein, 46 aa, Artificial Sequence
  Description of Artificial Sequence Synthetic peptide
KKKKKGAPGPPGDPGAPGPPGDPGAPGPPGDPGAPGPPGDPKKKKK

SEQ ID NO: 48 - protein, 46 aa, Artificial Sequence
  Description of Artificial Sequence Synthetic peptide
NNNNNGPAGPPGPAGPPGPAGPPGPAGPPGPAGPPGPAGPPNNNNN

SEQ ID NO: 49 - protein, 46 aa, Artificial Sequence
  Description of Artificial Sequence Synthetic peptide
NNNNNGAPGPPGAPGPPGAPGPPGAPGPPGAPGPPGAPGPPNNNNN

SEQ ID NO: 50 - protein, 46 aa, Artificial Sequence
  Description of Artificial Sequence Synthetic peptide
NNNNNGPVGPPGPVGPPGPVGPPGPVGPPGPVGPPGPVGPPNNNNN

SEQ ID NO: 51 - protein, 46 aa, Artificial Sequence
  Description of Artificial Sequence Synthetic peptide
NNNNNGVPGPPGVPGPPGVPGPPGVPGPPGVPGPPGVPGPPNNNNN

SEQ ID NO: 52 - protein, 46 aa, Artificial Sequence
  Description of Artificial Sequence Synthetic peptide
NGSNNGAPGPPGAPGPPGAPGPPGAPGPPGAPGPPGAPGPPNGSNN

SEQ ID NO: 53 - protein, 52 aa, Artificial Sequence
  Description of Artificial Sequence Synthetic peptide
NNNNNGPAGPPGPAGPPGPAGPPGPRGDPGAPGPPGAPGPPGAPGPPNNNNN

SEQ ID NO: 54 - protein, 46 aa, Artificial Sequence
  Description of Artificial Sequence Synthetic peptide
EEEEEGVPGPVGVPGPVGVPGPVGVPGPVGVPGPVGVPGPVEEEEE

SEQ ID NO: 55 - protein, 46 aa, Artificial Sequence
  Description of Artificial Sequence Synthetic peptide
EEEEEGVPGPAGVPGPAGVPGPAGVPGPAGVPGPAGVPGPAEEEEE

SEQ ID NO: 56 - protein, 6 aa, Artificial Sequence
  Description of Artificial Sequence Synthetic peptide
GAPGPP

SEQ ID NO: 57 - protein, 46 aa, Artificial Sequence; identical to SEQ ID NO: 46
  Description of Artificial Sequence Synthetic peptide

SEQ ID NO: 58 - protein, 46 aa, Artificial Sequence
  Description of Artificial Sequence Synthetic peptide
EEEEEGLPGPPGLPGPPGLPGPPGLPGPPGLPGPPGLPGPPEEEEE

SEQ ID NO: 59 - protein, 46 aa, Artificial Sequence
  Description of Artificial Sequence Synthetic peptide
EEEEEGIPGPPGIPGPPGIPGPPGIPGPPGIPGPPGIPGPPEEEEE

SEQ ID NO: 60 - protein, 46 aa, Artificial Sequence
  Description of Artificial Sequence Synthetic peptide
KKKKKGPPGPPGDPGPPGPPGDPGPPGPPGDPGPPGPPGDPKKKKK

SEQ ID NO: 61 - protein, 18 aa, Artificial Sequence
  Description of Artificial Sequence Synthetic peptide
SAASAASAASAASAASAA

SEQ ID NO: 62 - protein, 18 aa, Artificial Sequence
  Description of Artificial Sequence Synthetic peptide
SALSVASIASALSVASIA

SEQ ID NO: 63 - protein, 18 aa, Artificial Sequence
  Description of Artificial Sequence Synthetic peptide
YALSVATIAYALSVATIA

SEQ ID NO: 64 - protein, 20 aa, Artificial Sequence
  Description of Artificial Sequence Synthetic peptide
AAAAYAAAAYAAAAYAAAAY

SEQ ID NO: 65 - protein, 17 aa, Artificial Sequence
  Description of Artificial Sequence Synthetic peptide
AAAAYAAYAAYAAYAAY

SEQ ID NO: 66 - protein, 20 aa, Artificial Sequence
  Description of Artificial Sequence Synthetic peptide
AAASSIIIAAASIIIAAASS

SEQ ID NO: 67 - protein, 21 aa, Artificial Sequence
  Description of Artificial Sequence Synthetic peptide
EEEAAAAAAAAAAAAAAAEEE

SEQ ID NO: 68 - protein, 26 aa, Artificial Sequence
  Description of Artificial Sequence Synthetic peptide
EEEAAAAAAAAAAAAAAAAAAAAEEE

SEQ ID NO: 69 - protein, 28 aa, Artificial Sequence
  Description of Artificial Sequence Synthetic peptide
EEEAAAAAAAAAAAAAAAAAAAAAAEEE

SEQ ID NO: 70 - protein, 20 aa, Artificial Sequence
  Description of Artificial Sequence Synthetic peptide
EAAAKEAAAKEAAAKEAAAK

SEQ ID NO: 71 - protein, 25 aa, Artificial Sequence
  Description of Artificial Sequence Synthetic peptide
EAAAKEAAAKEAAAKEAAAKEAAAK

SEQ ID NO: 72 - protein, 6 aa, Artificial Sequence
  Description of Artificial Sequence Synthetic peptide
GIGIGS

SEQ ID NO: 73 - protein, 34 aa, Artificial Sequence
  Description of Artificial Sequence Synthetic peptide
EEEEEGAGAGDGAGAGDGAGAGDGAGAGDEEEEE

SEQ ID NO: 74 - protein, 34 aa, Artificial Sequence
  Description of Artificial Sequence Synthetic peptide
EEEEEGVGVGDGVGVGDGVGVGDGVGVGDEEEEE

SEQ ID NO: 75 - protein, 34 aa, Artificial Sequence
  Description of Artificial Sequence Synthetic peptide
EEEEEGLGLGDGLGLGDGLGLGDGLGLGDEEEEE

SEQ ID NO: 76 - protein, 34 aa, Artificial Sequence
  Description of Artificial Sequence Synthetic peptide
EEEEEGIGIGDGIGIGDGIGIGDGIGIGDEEEEE

SEQ ID NO: 77 - protein, 34 aa, Artificial Sequence
  Description of Artificial Sequence Synthetic peptide
EEEEEGVGVGYGVGVGYGVGVGYGVGVGYEEEEE

SEQ ID NO: 78 - protein, 34 aa, Artificial Sequence
  Description of Artificial Sequence Synthetic peptide
EEEEEGKGAGDGKGAGDGKGAGDGKGAGDEEEEE

SEQ ID NO: 79 - protein, 34 aa, Artificial Sequence
  Description of Artificial Sequence Synthetic peptide
EEEEEGAGAGTGAGAGTGAGAGTGAGAGTEEEEE

SEQ ID NO: 80 - protein, 34 aa, Artificial Sequence
  Description of Artificial Sequence Synthetic peptide
EEEEEGGAGGAGGAGGAGGAGGAGGAGGAEEEEE

SEQ ID NO: 81 - protein, 34 aa, Artificial Sequence
  Description of Artificial Sequence Synthetic peptide
EEEEEGGAGGTGGAGGTGGAGGTGGAGGTEEEEE

SEQ ID NO: 82 - protein, 34 aa, Artificial Sequence
  Description of Artificial Sequence Synthetic peptide
EEEEEGGAGGDGGAGGDGGAGGDGGAGGDEEEEE

SEQ ID NO: 83 - protein, 20 aa, Artificial Sequence
  Description of Artificial Sequence Synthetic peptide
SGAGVGRGDGSGVGLGSGNG

SEQ ID NO: 84 - protein, 12 aa, Artificial Sequence
  Description of Artificial Sequence Synthetic peptide
GDVGGAGATGGS

SEQ ID NO: 85 - protein, 12 aa, Artificial Sequence
  Description of Artificial Sequence Synthetic peptide
GNVGGAGASGGS

SEQ ID NO: 86 - protein, 15 aa, Artificial Sequence
  Description of Artificial Sequence Synthetic peptide
GGAGGGGTGGLGSGG

SEQ ID NO: 87 - protein, 27 aa, Artificial Sequence
  Description of Artificial Sequence Synthetic peptide
GGAGGGGTGGLGSGGAGAGGLGGGGAG

SEQ ID NO: 88 - protein, 16 aa, Artificial Sequence
  Description of Artificial Sequence Synthetic peptide
GPGGTGPGQQGPGGTW

SEQ ID NO: 89 - protein, 23 aa, Artificial Sequence
  Description of Artificial Sequence Synthetic peptide
GPGNNGPGGYGPGNNGPSGPGSA

SEQ ID NO: 90 - protein, 26 aa, Artificial Sequence
  Description of Artificial Sequence Synthetic peptide
DPGVYGPSGNAPGVYGPSGQGAGAGS

SEQ ID NO: 91 - protein, 34 aa, Artificial Sequence
  Description of Artificial Sequence Synthetic peptide
EEEEEGAGAGSGAGAGSGAGAGSGAGAGSEEEEE

SEQ ID NO: 92 - protein, 34 aa, Artificial Sequence
  Description of Artificial Sequence Synthetic peptide
EEEEEGVGVGSGVGVGSGVGVGSGVGVGSEEEEE

SEQ ID NO: 93 - protein, 34 aa, Artificial Sequence
  Description of Artificial Sequence Synthetic peptide
EEEEEGIGIGSGIGIGSGIGIGSGIGIGSEEEEE

SEQ ID NO: 94 - protein, 34 aa, Artificial Sequence; identical to SEQ ID NO: 77
  Description of Artificial Sequence Synthetic peptide

SEQ ID NO: 95 - protein, 34 aa, Artificial Sequence
  Description of Artificial Sequence Synthetic peptide
EEEEEGAGAGYGAGAGYGAGAGYGAGAGYEEEEE

SEQ ID NO: 96 - protein, 34 aa, Artificial Sequence; identical to SEQ ID NO: 73
  Description of Artificial Sequence Synthetic peptide

SEQ ID NO: 97 - protein, 30 aa, Artificial Sequence
  Description of Artificial Sequence Synthetic peptide
EEEEGAGAGSGAGAGSEEEEGAGAGSEEEE

SEQ ID NO: 98 - protein, 24 aa, Artificial Sequence
  Description of Artificial Sequence Synthetic peptide
EEEEGAGAGSEEEEGAGAGSEEEE

SEQ ID NO: 99 - protein, 30 aa, Artificial Sequence
  Description of Artificial Sequence Synthetic peptide
EEEEEEGAGAGDGAGAGDGAGAGDEEEEEE

SEQ ID NO: 100 - protein, 30 aa, Artificial Sequence; identical to SEQ ID NO: 99
  Description of Artificial Sequence Synthetic peptide

SEQ ID NO: 101 - protein, 30 aa, Artificial Sequence
  Description of Artificial Sequence Synthetic peptide
EEEEEEGIGIGSGIGIGSGIGIGSEEEEEE

SEQ ID NO: 102 - protein, 30 aa, Artificial Sequence
  Description of Artificial Sequence Synthetic peptide
EEEEEEGVGVGYGVGVGYGVGVGYEEEEEE

SEQ ID NO: 103 - protein, 30 aa, Artificial Sequence
  Description of Artificial Sequence Synthetic peptide
EEEEEEGAGAGSGAGAGSGAGAGSEEEEEE

SEQ ID NO: 104 - protein, 25 aa, Artificial Sequence
  Description of Artificial Sequence Synthetic peptide
EEEEEGPGGYGPGQQGPGGYEEEEE

SEQ ID NO: 105 - protein, 33 aa, Artificial Sequence
  Description of Artificial Sequence Synthetic peptide
EEEEEGPGQQGPGGYGPGQQGPSGPGSAEEEEE

SEQ ID NO: 106 - protein, 30 aa, Artificial Sequence
  Description of Artificial Sequence Synthetic peptide
EEEEEDPGVYGPSGQDPGVYGPSGQEEEEE

SEQ ID NO: 107 - protein, 12 aa, Artificial Sequence
  Description of Artificial Sequence Synthetic peptide
GAIGGVGATGGS

SEQ ID NO: 108 - protein, 33 aa, Artificial Sequence
  Description of Artificial Sequence Synthetic peptide
RRRGGAGQGGYGGLGSQGAGRGGLGGQGAGRRR

SEQ ID NO: 109 - protein, 6 aa, Artificial Sequence
  Description of Artificial Sequence Synthetic peptide
GVGVGS

SEQ ID NO: 110 - protein, 50 aa, Artificial Sequence; identical to SEQ ID NO: 1
  Description of Artificial Sequence Synthetic peptide

SEQ ID NO: 111 - protein, 34 aa, Artificial Sequence
  Description of Artificial Sequence Synthetic peptide
EEEEEGDIGGVGATGGSGDIGGVGATGGSEEEEE

SEQ ID NO: 112 - protein, 34 aa, Artificial Sequence
  Description of Artificial Sequence Synthetic peptide
EEEEEGNVGGAGASGGSGNVGGAGASGGSEEEEE

SEQ ID NO: 113 - protein, 34 aa, Artificial Sequence
  Description of Artificial Sequence Synthetic peptide
EEEEEGVDGGAGATGGSGVDGGAGATGGSEEEEE

SEQ ID NO: 114 - protein, 6 aa, Artificial Sequence
  Description of Artificial Sequence Synthetic peptide
GVGGGY

SEQ ID NO: 115 - protein, 6 aa, Artificial Sequence
  Description of Artificial Sequence Synthetic peptide
GAGAGY

SEQ ID NO: 116 - protein, 6 aa, Artificial Sequence
  Description of Artificial Sequence Synthetic peptide
GAGAGD

SEQ ID NO: 117 - protein, 27 aa, Artificial Sequence
  Description of Artificial Sequence Synthetic peptide
SGRGGLGGQGAGGGAGQGGYGGLGSQG

SEQ ID NO: 118 - protein, 6 aa, Artificial Sequence
  Description of Artificial Sequence Synthetic peptide
GAGAGT

SEQ ID NO: 119 - protein, 20 aa, Artificial Sequence
  Description of Artificial Sequence Synthetic peptide
SGAGVGRGDGSGVGLGSGNG

SEQ ID NO: 120 - protein, 33 aa, Artificial Sequence; identical to SEQ ID NO: 108
  Description of Artificial Sequence Synthetic peptide

SEQ ID NO: 121 - protein, 34 aa, Artificial Sequence
  Description of Artificial Sequence Synthetic peptide
EEEEEGDVGGAGATGGSGDVGGAGATGGSEEEEE

SEQ ID NO: 122 - protein, 34 aa, Artificial Sequence
  Description of Artificial Sequence Synthetic peptide
EEEEEGDVGGAGASGGSGDVGGAGASGGSEEEEE

SEQ ID NO: 123 - protein, 34 aa, Artificial Sequence; identical to SEQ ID NO: 111
  Description of Artificial Sequence Synthetic peptide

SEQ ID NO: 124 - protein, 25 aa, Artificial Sequence
  Description of Artificial Sequence Synthetic peptide
EEEEEGPGGYGPGQQGPGGYEEEEE

SEQ ID NO: 125 - protein, 33 aa, Artificial Sequence; identical to SEQ ID NO: 105
  Description of Artificial Sequence Synthetic peptide

SEQ ID NO: 126 - protein, 30 aa, Artificial Sequence; identical to SEQ ID NO: 106
  Description of Artificial Sequence Synthetic peptide

SEQ ID NO: 127 - protein, 34 aa, Artificial Sequence
  Description of Artificial Sequence Synthetic peptide
SGRGGLGGQGAGMAAAAAMGGAGQGGYGGLGSQG

SEQ ID NO: 128 - protein, 24 aa, Artificial Sequence
  Description of Artificial Sequence Synthetic peptide
MKHMAGAAGAVVGGLGGYMLGSAM

SEQ ID NO: 129 - protein, 52 aa, Artificial Sequence
  Description of Artificial Sequence Synthetic peptide
MDAEFRHDSGYEVHHQKLVFFAEDVGSNKGAIIGLMVGGVVIATVIVITLVM

SEQ ID NO: 130 - protein, 46 aa, Artificial Sequence
  Description of Artificial Sequence Synthetic peptide
EEEEVSSTGSTSNTDSSSKSAGSRTSGGTSTYGYSSSHRGGSEEEE

SEQ ID NO: 131 - protein, 7 aa, Artificial Sequence
  Description of Artificial Sequence Synthetic peptide
DEDEDED

SEQ ID NO: 132 - protein, 18 aa, Artificial Sequence
  Description of Artificial Sequence Synthetic peptide
GAGAGSGAGAGSGAGAGS

SEQ ID NO: 133 - protein, 6 aa, Artificial Sequence
  Description of Artificial Sequence Synthetic peptide
GAPGPP

SEQ ID NO: 134 - protein, 6 aa, Artificial Sequence
  Description of Artificial Sequence Synthetic peptide
GPAGPP

SEQ ID NO: 135 - protein, 6 aa, Artificial Sequence
  Description of Artificial Sequence Synthetic peptide
GPAGPP

SEQ ID NO: 136 - protein, 6 aa, Artificial Sequence
  Description of Artificial Sequence Synthetic peptide
GAPGPP

SEQ ID NO: 137 - protein, 6 aa, Artificial Sequence
  Description of Artificial Sequence Synthetic peptide
GAPGPP

SEQ ID NO: 138 - protein, 46 aa, Artificial Sequence; identical to SEQ ID NO: 54
  Description of Artificial Sequence Synthetic peptide

SEQ ID NO: 139 - protein, 46 aa, Artificial Sequence; identical to SEQ ID NO: 55
  Description of Artificial Sequence Synthetic peptide

SEQ ID NO: 140 - protein, 46 aa, Artificial Sequence; identical to SEQ ID NO: 58
  Description of Artificial Sequence Synthetic peptide

SEQ ID NO: 141 - protein, 46 aa, Artificial Sequence; identical to SEQ ID NO: 59
  Description of Artificial Sequence Synthetic peptide

SEQ ID NO: 142 - protein, 46 aa, Artificial Sequence; identical to SEQ ID NO: 60
  Description of Artificial Sequence Synthetic peptide

SEQ ID NO: 143 - protein, 46 aa, Artificial Sequence; identical to SEQ ID NO: 47
  Description of Artificial Sequence Synthetic peptide

SEQ ID NO: 144 - protein, 6 aa, Artificial Sequence
  Description of Artificial Sequence Synthetic peptide
GSPGPP

SEQ ID NO: 145 - protein, 6 aa, Artificial Sequence
  Description of Artificial Sequence Synthetic peptide
GAGAGS

SEQ ID NO: 146 - protein, 10 aa, Artificial Sequence
  Description of Artificial Sequence Synthetic peptide
GXGXGXGXGX

SEQ ID NO: 147 - protein, 15 aa, Artificial Sequence
  Description of Artificial Sequence Synthetic peptide
GGXGGXGGXGGXGGX

SEQ ID NO: 148 - protein, 10 aa, Artificial Sequence
  Description of Artificial Sequence Synthetic peptide
XGXGXGXGXG

SEQ ID NO: 149 - protein, 15 aa, Artificial Sequence
  Description of Artificial Sequence Synthetic peptide
XGGXGGXGGXGGXGG

SEQ ID NO: 150 - protein, 15 aa, Artificial Sequence
  Description of Artificial Sequence Synthetic peptide
GXXGXXGXXGXXGXX

SEQ ID NO: 151 - protein, 15 aa, Artificial Sequence
  Description of Artificial Sequence Synthetic peptide
XXGXXGXXGXXGXXG

SEQ ID NO: 152 - protein, 30 aa, Artificial Sequence
  Description of Artificial Sequence Synthetic peptide
GGXGXXGGXGXXGGXGXXGGXGXXGGXGXX

SEQ ID NO: 153 - protein, 50 aa, Artificial Sequence
  Description of Artificial Sequence Synthetic peptide
GXGXGGXGXXGXGXGGXGXXGXGXGGXGXXGXGXGGXGXXGXGXGGXGXX

SEQ ID NO: 154 - protein, 30 aa, Artificial Sequence
  Description of Artificial Sequence Synthetic peptide
GSPGPPGSPGPPGSPGPPGSPGPPGSPGPP

SEQ ID NO: 155 - protein, 30 aa, Artificial Sequence
  Description of Artificial Sequence Synthetic peptide
GAGAGSGAGAGSGAGAGSGAGAGSGAGAGS

SEQ ID NO: 156 - protein, 46 aa, Artificial Sequence
  Description of Artificial Sequence Synthetic peptide
EEEEEGPAGPPGPAGPPGPAGPPGPAGPPGPAGPPGPAGPPEEEEE

SEQ ID NO: 157 - protein, 6 aa, Artificial Sequence
  Description of Artificial Sequence Synthetic peptide
GVPGPP

SEQ ID NO: 158 - protein, 94 aa, Artificial Sequence
  Description of Artificial Sequence Synthetic peptide
NYNSGCCCGGCCCGGCCCGNSYSGPGGTGPGQQGPGGTWGPGGTGPGQQGPGGTWGPGGVGVGSGAGAGDGVGVGSGAGAGDGVGVGSGAGAGD

SEQ ID NO: 159 - protein, 36 aa, Artificial Sequence
  Description of Artificial Sequence Synthetic peptide
GEGPMKHMAEGPGGAGAGYGAGYGPGGESYRGDGSG

SEQ ID NO: 160 - protein, 36 aa, Artificial Sequence
  Description of Artificial Sequence Synthetic peptide
GPGGVPGVAGGPGVPGVAGGPGVPGVAGGPSYSSNR

SEQ ID NO: 161 - protein, 46 aa, Artificial Sequence
  Description of Artificial Sequence Synthetic peptide
EEEEEGSPGPPGSPGPPGSPGPPGSPGPPGSPGPPGSPGPPEEEEE

SEQ ID NO: 162 - protein, 15 aa, Artificial Sequence
  Description of Artificial Sequence Synthetic peptide
XGXGXGXGXGGGXGX

SEQ ID NO: 163 - protein, 15 aa, Artificial Sequence
  Description of Artificial Sequence Synthetic peptide
XGAGVGSGDGXGVGL